SEQUENCE LISTING (22 sequences)

SEQ ID NO: 1 - protein, 12 aa, Homo sapiens
  Spacer (IgG4hinge)
ESKYGPPCPPCP

SEQ ID NO: 2 - DNA, 36 bp, Homo sapiens
  Spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28
FWVLVVVGGVLACYSLLVTVAFIIFWV

SEQ ID NO: 9 - protein, 66 aa, Homo sapiens
  CD28
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28
RSKRSRLLHSDYMNMTPRRPGPTRKHYQPYAPPRDFAAYRS

SEQ ID NO: 11 - protein, 41 aa, Homo sapiens
  CD28
RSKRSRGGHSDYMNMTPRRPGPTRKHYQPYAPPRDFAAYRS

SEQ ID NO: 12 - protein, 42 aa, Homo sapiens
  4-1BB
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 10 aa, Artificial Sequence
  Linker
  REPEAT (5)...(9): SGGGG is repeated 5 times
PGGGSGGGGP

SEQ ID NO: 17 - protein, 17 aa, Artificial Sequence
  Linker
GSADDAKKDAAKKDGKS

SEQ ID NO: 18 - protein, 18 aa, Artificial Sequence
  T2A
EGRGSLLTCGDVEENPGP

SEQ ID NO: 19 - protein, 22 aa, Artificial Sequence
  P2A
GSGATNFSLLKQAGDVEENPGP

SEQ ID NO: 20 - protein, 19 aa, Artificial Sequence
  P2A
ATNFSLLKQAGDVEENPGP

SEQ ID NO: 21 - protein, 20 aa, Artificial Sequence
  E2A
QCTNYALLKLAGDVESNPGP

SEQ ID NO: 22 - protein, 22 aa, Artificial Sequence
  F2A
VKQTLNFDLLKLAGDVESNPGP

(19) United States
(12) Patent Application Publication
(10) Pub. No.: US 2019/0292246 A1
(43) Pub. Date: Sep. 26, 2019

(54) COMBINATION THERAPY OF A CELL BASED THERAPY AND A MICROGLIA IMHIBITOR

(71) Applicant: JUNO THERAPEUTICS, INC., Seattle, WA (US)

(72) Inventor: Hyam I. LEVITSKY, Seattle, WA (US)

(73) Assignee: JUNO THERAPEUTICS, INC., Seattle, WA (US)

(21) Appl. No.: 16/345,214

(22) PCT Filed: Nov. 3, 2017
(86) PCT No.: PCT/US2017/060058

§ 371 Date: Apr. 25, 2019

Related U.S. Application Data
(60) Provisional application No. 62/527,028, filed on Jun. 29, 2017.
(60) Provisional application No. 62/429,713, filed on Dec. 2, 2016.
(60) Provisional application No. 62/417,318, filed on Nov. 3, 2016.
(60) Provisional application No. 62/417,315, filed on Nov. 3, 2016.

(51) Int. Cl. C07K 16/18; A61K 31/444; A61K 31/675; A61K 9/00; A61K 35/17

(52) U.S. Cl. CPC C07K 16/18; A61K 31/444; A61K 45/06; A61K 9/0019; A61K 35/17; A61K 31/675

(57) ABSTRACT

Provided are methods, kits and compositions for ameliorating toxicity, such as cytokine release syndrome or neurotoxicity, suspected or being induced by or associated with, administration of a therapeutic agent, such as a immunotherapeutic agent targeting T cells and/or genetically engineered T cells, e.g. chimeric antigen receptor (CAR)-expressing T cells. The methods involve administering an additional agent, such as an agent having anti-oxidant or anti-inflammatory properties, that modulates immune cells such as by preventing or reducing the production of pro-inflammatory cytokines or stress cytokines and/or promoting differentiation to a neuroprotective phenotype, and/or capable of preventing, blocking or reducing microglial cell activation or function and/or capable of modulating, such as promoting, the activity of NRF2 or a component of an NRF2-regulated pathway, and/or one or more components involved in an antioxidant response element (ARE). The provided methods can be used in connection with or for methods of treating a disease or condition.

### CROSS-REFERENCE TO RELATED APPLICATIONS

This application claims priority from U.S. provisional application No. 62/417,315, filed Nov. 3, 2016, entitled “Combination Therapy of a Cell Based Therapy and a Microglia Inhibitor,” U.S. provisional application No. 62/417,318, filed Nov. 3, 2016, entitled “Combination Therapy of a Cell Based Therapy and a Microglia Inhibitor,” U.S. provisional application No. 62/429,713 filed Dec. 2, 2016 entitled “Combination Therapy of a Cell Based Therapy and a Microglia Inhibitor,” and U.S. provisional application No. 62/527,028 filed Jun. 29, 2017, entitled “Combination Therapy of a Cell Based Therapy and a Microglia Inhibitor,” the contents of each of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042007140SeqList.TXT, created Oct. 23, 2017 which is 16,852 bytes in size. The information in the electronic format of the Sequence Listing is incorporated by reference in its entirety.

### FIELD

The present disclosure provides methods, kits and compositions for ameliorating toxicity induced by or associated with, or suspected or being induced by or associated with, administration of a therapeutic agent. In some embodiments, the toxicity is a neurotoxicity or cytokine release syndrome (CRS), such as a severe neurotoxicity or a severe CRS. In some embodiments, the methods involved administration to a subject having a disease of condition a therapeutic agent for treating the disease or condition and an additional agent, such as an agent having anti-oxidant or anti-inflammatory properties, an agent that modulates immune cells such as by preventing or reducing the production of pro-inflammatory cytokines or stress cytokines and/or promoting differentiation thereof to a neuroprotective phenotype, and/or an agent capable of preventing, blocking or reducing microglial cell activation or function and/or an agent capable of modulating, such as promoting, the activity or function of NRF2 or a component of an NRF2-regulated pathway, and/or one or more genes or components involved in an antioxidant response element (ARE). In some embodiments, the provided methods can be used in connection with or for methods of treating a disease or condition. In some embodiments, the therapeutic agent is an immunotherapeutic agent targeting T cells, such as a therapeutic antibody, e.g., a multispecific (e.g., T cell engaging) antibody, and/or genetically engineered T cells, such as chimeric antigen receptor (CAR)-expressing T cells. In some embodiments, the agent is an inhibitor of colony stimulating factor 1 receptor (CSF1R). In some aspects, the agent is or comprises a DMF. Also provided are articles of manufacture and kits for use in the methods.

### BACKGROUND

Various methods are available for adoptive cell therapy using engineered cells expressing recombinant receptors, such as chimeric antigen receptor (CARs). Improved methods are needed, for example, to reduce the risk of toxicity and/or to increase efficacy, for example, by increasing exposure of the subject to the administered cells, for example, by improving expansion and/or persistence of the administered cells. Provided are methods, compositions, and articles of manufacture that meet such needs.

### SUMMARY

Provided herein is a method of treatment including administering to a subject having a disease or condition a therapeutic agent for treating a disease or condition, wherein administration of the therapeutic agent is or is suspected of being associated with a risk of eliciting a toxic outcome or symptom; and the subject has been administered, prior to initiation of the therapy, an agent capable of modulating one or more function or activity of immune cells such as microglial cells or astrocytes, such as an agent capable of preventing, blocking or reducing an activity or function of microglial cell activity or function and/or promoting an alternative function or phenotype thereof, such as promoting a neuroprotective phenotype thereof. In some aspects, the agent is or comprises an agent having anti-oxidant or anti-inflammatory properties, an agent that modulates immune cells such as by preventing or reducing the production of pro-inflammatory cytokines or stress cytokines and/or promoting differentiation thereof to a neuroprotective phenotype. In some embodiments, the prior administration of the agent is in an amount effective to prevent, block or reduce microglial cell activity or function in the subject. In some embodiments, the method further includes prior to administering the therapy, administering to the subject the agent capable of that preventing, blocking or reducing microglial cell activity.

Also provided is a method of treatment including (a) administering to a subject an agent capable of preventing, blocking or reducing microglial cell activity or function; and (b) after the administration in (a), administering to the subject having a disease or condition a therapeutic agent for treating a disease or condition, wherein administration of the therapeutic agent is or is suspected of being associated with a risk of eliciting a toxic outcome or symptom. In some cases, the agent is administered in an amount effective to prevent, block or reduce microglial cell activity or function in the subject.

In some of any such embodiments, the toxic outcome or symptom is associated with neurotoxicity or cytokine release syndrome (CRS).

In some of any such embodiments, the toxic outcome or symptom is associated with severe neurotoxicity and/or is associated with grade 2 or higher or grade 3 or higher neurotoxicity; and/or the toxic outcome or symptom is associated with severe CRS and/or is associated with grade 2 or higher or grade 3 or higher CRS. In some of any such embodiments, the toxic outcome is cerebral edema or is associated with cerebral edema.

In some of any such embodiments, administration of the agent is started at a time point that is within or within about 1 hour, 2 hours, 6 hours, 12 hours, 24 hours, 3 days, 6 days, 12 days, 15 days, 30 days, 60 days or 90 days or more prior to administration of the therapy. In some of any such embodiments, the agent is administered greater than 4 days prior to initiation of the therapy.

In some of any such embodiments, the therapy is not or does not contain interleukin 2 (IL-2); the subject has not previously received administration of IL-2 prior to administration of the therapy; or the subject has not received administration of IL-2 greater than 4 days prior to initiation of the therapy.

In some of any such embodiments, the agent is not further administered after administration of the therapeutic agent.

In some of any such embodiments, the method further includes administering the agent concurrently with or after administration of the therapeutic agent. In some aspects, the agent is administered within or within about 1 day, 2 days, 3 days, four days, five days, six days or seven days after administration of the therapeutic agent.

Also provided herein is a method of treatment including (a) administering to a subject having a disease or condition a therapeutic agent for treating a disease or condition, wherein administration of the therapeutic agent is or is suspected of being associated with a risk of eliciting a toxic outcome or symptom of or related to severe CRS or severe neurotoxicity in the subject and/or grade 2 or grade 3 or higher CRS or grade 2 or grade 3 or higher neurotoxicity in the subject; and (b) administering to the subject an agent capable of preventing, blocking or reducing microglial cell activity or function, wherein the agent is administered (i) at a time that is within or within about 1 day, 2 days, 3 days, four days, five days, six days or seven days after administration of the therapeutic agent and/or (ii) at or about or within 24 hours of the subject exhibiting a first sign or symptom indicative of CRS or neurotoxicity after administration of the therapy. In some aspects, the agent is administered in an amount effective to prevent, block or reduce microglial cell activity or function in the subject. In some instances, the first sign or symptom indicative of CRS or neurotoxicity is a fever.

Also provided herein is a method of treatment including (a) administering to a subject having a disease or condition a therapeutic agent for treating a disease or condition, wherein the therapeutic agent is or is suspected of being associated with a risk of eliciting a toxic outcome or symptom; and (b) administering to the subject an agent capable of preventing, blocking or reducing microglial cell activity or function or presence, wherein the agent is administered at or about or within 24 hours of the subject exhibiting a fever after administration of the therapeutic agent. In some examples, the agent is administered in an amount effective to prevent, block or reduce microglial cell activity or function in the subject.

In some of any such embodiments, the fever contains a temperature of at least or at least about 38.0° C. In some embodiments, the fever contains a temperature that is between or between about 38.0° C. and 42.0° C., 38.0° C. and 39.0° C., 39.0° C. and 40.0° C. or 40.0° C. and 42.0° C., each inclusive; or the fever contains a temperature that is greater than or greater than about or is or is about 38.5° C., 39.0°, 39.5° C., 40.0° C., 41.0° C., 42.0° C. In some cases, the fever is a sustained fever. In some of any such embodiments, the fever is a fever that is not reduced or not reduced by more than 1° C. after treatment with an antipyretic and/or wherein the fever has not been reduced by more than 1° C., following treatment of the subject with an antipyretic.

In some aspects, the first sign or symptom indicative of CRS or neurotoxicity is an altered level of one or more biomarkers in a sample from the subject compared to in the sample prior to administration of the therapeutic agent. In some cases, the sample is a serum or blood sample. In some of any such embodiments, the sample is obtained or has been obtained from the subject no more than 3 days, no more than 2 days or no more than 1 day after initiation of the therapy or a first administration of the therapeutic agent. In some of any such embodiments, the altered level is an increased level of the one or more biomarker, optionally increased greater than or greater than about 1.5-fold, 2-fold, 3-fold, 4-fold, 5-fold, 10-fold, 20-fold or 50-fold.

In some of any such embodiments, the method further includes assessing the sample from the subject for the one or more biomarkers after administration of the cell therapy and prior to administration of the agent. In some of any such embodiments, administration of the agent is continued after initiation of administration of the therapeutic agent until the risk or suspected risk of a toxic outcome or symptom in the subject from administration of the therapeutic agent has subsided or is not present.

Also provided is a method of ameliorating toxicity induced by or associated with administration of a therapeutic agent, the method including (a) administering to a subject having a disease or condition a therapeutic agent for treating a disease or condition, wherein the therapeutic agent is or is suspected of being associated with a risk of eliciting a toxic outcome or symptom; and (b) administering to the subject an agent capable of preventing, blocking or reducing microglial cell activation or function, wherein the agent is administered in a dosage regimen until the risk or suspected risk of a toxic outcome or symptom associated with administration of the therapeutic agent has subsided or is not present. In some examples, the agent is administered in an amount effective to prevent, block or reduce microglial cell activity or function in the subject. In some embodiments, the agent is administered prior to, simultaneously with and/or subsequent to initiation of administration of the therapeutic agent.

In some of any such embodiments, the agent is administered for a time period up to 2 days, up to 7 days, up to 14 days, up to 21 days, up to 28 days, up to 35 days, up to 42 days, up to two months, up to three months, up to 6 months or up to 1 year after initiation of the administration of the therapeutic agent.

In some of any such embodiments, the agent is administered for a time period until the grade of CRS or neurotoxicity in the subject is reduced to a lower grade compared to prior to administration of the agent or compared to a preceding time point after administration of the agent or a sign or symptom of grade 1 or higher or grade 2 or higher CRS or neurotoxicity is not present or detectable in the subject after administration of the agent.

In some of any such embodiments, prior to the administration, the subject has been preconditioned with a lymphodepleting therapy containing one or more chemotherapeutic agent. In some of any such embodiments, the method further includes prior to the administration of the therapeutic agent, administering to the subject a lymphodepleting therapy containing one or more chemotherapeutic agent.

In some of any such embodiments, the chemotherapeutic agent contains an agent selected from the group consisting of cyclophosphamide, fludarabine, and/or a combination thereof. In some aspects, the chemotherapeutic agent is or contains fludarabine that is administered at a dose of between or between about 1 mg/m² and 100 mg/m², between or between about 10 mg/m² and 75 mg/m², between or between about 15 mg/m² and 50 mg/m², between or between about 20 mg/m² and 30 mg/m², or between or between about 24 mg/m² and 26 mg/m²; and/or the chemotherapeutic agent is cyclophosphamide that is administered between or between about 20 mg/kg and 100 mg/kg, between or between about 40 mg/kg and 80 mg/kg or between or between about 30 mg/kg and 60 mg/kg. In some cases, the cyclophosphamide is administered once daily for one or two days, and/or the fludarabine is administered daily for 3-5 days.

In some of any such embodiments, the lymphodepleting therapy contains administration of cyclophosphamide between or between about 30 mg/kg and 60 mg/kg and administration of fludarabine between or between about 25 mg/m² and 30 mg/m² for three days. In some of any such embodiments, the lymphodepleting therapy is initiated at a time that is at least at or about 2 days prior to or is between at or about 2 days and at or about 7 days prior to the administration of the therapeutic agent.

In some of any such embodiments, the therapeutic agent is an immunotherapy. In some of any such embodiments, the therapeutic agent is a T cell therapy or is a T cell-engaging therapy. In some aspects, the therapeutic agent is a T cell-engaging therapy containing a bispecific antibody, wherein at least one binding portion specifically binds to a T cell antigen, optionally CD3. In some cases, the cell therapy is an adoptive cell therapy.

In some of any such embodiments, the therapeutic agent is a T cell therapy that is or includes tumor infiltrating lymphocytic (TIL) therapy or a T cell therapy including genetically engineered cells expressing a recombinant receptor that specifically binds to a ligand. In some embodiments, the T cell therapy is or includes genetically engineered cells expressing a recombinant receptor that specifically binds to a ligand.

In some of any such embodiments, the agent capable of preventing, blocking or reducing microglial cell activity reduces the expression of a microglial activation marker on microglial cells, reduces the level or amount one or more effector molecule associated with microglial cell activation in a biological sample; alters microglial cell homeostasis; decreases or blocks microglial cell proliferation; and/or reduces or eliminates microglial cells.

In some of any such embodiments, the agent reduces or eliminates microglial cells and the reduction in the number of microglial cells is by greater than 20%, greater than 30%, greater than 40% or greater than 50%, greater than 60%, greater than 70%, greater than 80%, greater than 90%, greater than 95% or greater than 99% compared to the number of microglial cells at a time just prior to initiation of the administration of the agent.

In some of any such embodiments, the agent reduces the expression of a microglial activation marker, optionally CD86 and CD68; and/or the agent reduces the level or amount of one or more effector molecule, wherein the one or more effector molecule is a optionally or one or more pro-inflammatory mediator, optionally selected from one or more of inducible nitric oxide synthase (iNOS), prostaglandin E(2) (PGE(2)), IL-6, IL-1β, IL-8, CCL2, CXCL10, TNF-α, CCL7, CXCL5, CXCL9, CXCL6, MMP-7, MMP-2, and MMP-9. In some of any such embodiments, the biological sample is a brain, serum or plasma sample.

In some of any such embodiments, the agent, such as the agent that reduces microglial cell activation, is selected from an anti-inflammatory agent, an inhibitor of NADPH oxidase (NOX2), a calcium channel blocker, a sodium channel blocker, inhibits GM-CSF, inhibits CSF1R, specifically binds CSF-1, specifically binds IL-34, inhibits the activation of nuclear factor kappa B (NF-κB), activates a CB₂ receptor and/or is a CB₂ agonist, a phosphodiesterase inhibitor, inhibits microRNA-155 (miR-155) or upregulates microRNA-124 (miR-124).

In some of any such embodiments, the prevention, block or reduction of microglial cell activation or function by the agent is transient and/or is reversible upon discontinued administration of the agent.

In some of any such embodiments, the agent, such as the agent that is capable of preventing, blocking or reducing microglial cell activation or function, is a small molecule, peptide, protein, antibody or antigen-binding fragment thereof, an antibody mimetic, an aptamer, or a nucleic acid molecule. In some examples, the agent is selected from minocycline, naloxone, nimodipine, Riluzole, MOR103, lenalidomide, a cannabinoid (optionally WIN55 or 212-2), intravenous immunoglobulin (IVIg), ibudilast, anti-miR-155 locked nucleic acid (LNA), MCS110, PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945, emactuzumab, IMC-CS4, FPA008, LY-3022855, AMG-820 and TG-3003.

In some of any such embodiments, the agent is an inhibitor of colony stimulating factor 1 receptor (CSF1R). In some cases, the inhibitor transiently inhibits the activity of CSF1R and/or wherein the inhibition of CSF1R activity is not permanent.

In some of any such embodiments, the inhibitor is selected from PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof, emactuzumab, IMC-CS4, FPA008, LY-3022855, AMG-820 and TG-3003 or a combination of any of the foregoing. In some of any such embodiments, the inhibitor is PLX-3397.

In some embodiments, the agent is an inhibitor of nitric oxide synthase. In some embodiments, the inhibitor of nitric oxide synthase is selected from VAS-203, cindunistat, A-84643, ONO-1714, L-NOARG, NCX-456, VAS-2381, GW-273629, NXN-462, CKD-712, KD-7040, and guanidinoethyldisulfide.

In some embodiments, the agent is an activator or upregulator of NRF2 or an NRF2-regulated or -related pathway. In some embodiments, the agent and/or the activator of NRF2 or an NRF2-regulated or -related pathway is dimethyl fumarate (DMF).

In some embodiments, the agent sequesters T cells from the central nervous system.

In some embodiments, the agent modulates a sphingosine-1-phosphate (S1P) receptor. In some embodiments, the S1P receptor is a S1PR1 and/or a S1PR5.

In some embodiments, the agent is fingolimod (Gilenya®) or ozanimod (RPC-1063).

Also provided herein is a method of treatment including administering to a subject having a disease or condition, a cell therapy for treating a disease or condition, wherein the cell therapy contains cells that secrete an inhibitor of colony-stimulating factor-1 receptor (CSF1R). In some cases, the cell therapy is a T cell therapy. In some instances, the inhibitor is a peptide, polypeptide or antibody or antigen-binding fragment thereof. In some cases, the inhibitor is an antibody or antigen-binding fragment thereof. In some embodiments, the inhibitor is selected from emactuzumab, IMC-CS4, FPA008, LY-3022855, AMG-820, TG-3003 or is an antigen-binding fragment thereof.

In some of any such embodiments, the therapeutic agent is administered after administering the agent at a time at which microglial cell activation or function is reduced, blocked or prevented or is likely to be reduced, blocked or prevented in the subject or at a time in which a parameter associated with activity of the agent is altered in the subject.

In some of any such embodiments, the therapeutic agent is administered after administering the agent at a time at which: (i) the number of microglial cells is reduced or eliminated in the subject compared to just prior to initiation of administration of the agent; or (ii) there exists a reduction in the level or amount of a proinflammatory mediator of microglial cell activation in a sample, optionally a brain, serum or plasma sample, from the subject compared to just prior to initiation of administration of the agent; (iii) the expression of a microglial cell activation marker, optionally CD86 or CD68, is reduced compared to just prior to initiation of administration of the agent; (iv) there is an increase in the plasma or serum level of CSF-1 or IL-34 compared to just prior to initiation of administration of the agent; (v) there is a reduction of Kupffer cells and/or an increase in the level or amount of a serum enzyme associated with reduction of Kupffer cells compared to just prior to initiation of administration of the agent; (vi) there is a reduction in the number of tumor-associated macrophages (TAM) compared to just prior to initiation of administration of the agent; and/or (vii) there is a decrease in CD14dim/CD16+ nonclassical monocytes in peripheral blood compared to just prior to initiation of administration of the agent.

In some of any such embodiments, the method further includes after administering the agent but prior to administering the therapeutic agent assessing a sample from the subject for a prevention, block or reduction in microglial cell activation or function or for alteration of a parameter associated with activity of the agent.

In some of any such embodiments, the method further includes after administering the agent but prior to administering the therapeutic agent assessing a sample from the subject for one or more of: (i) a reduction or elimination of microglial cells in the subject compared to just prior to initiation of administration of the agent; or (ii) a reduction in the level or amount of a proinflammatory mediator of microglial cell activation in a sample, optionally a brain, serum or plasma sample, from the subject compared to just prior to initiation of administration of the agent; (iii) a reduction in expression of a microglial cell activation marker, optionally CD86 or CD68, compared to just prior to initiation of administration of the agent; (iv) an increase in the plasma or serum level of CSF-1 or IL-34 compared to just prior to initiation of administration of the agent; (v) a reduction of Kupffer cells and/or an increase in the level or amount of a serum enzyme associated with reduction of Kupffer cells compared to just prior to initiation of administration of the agent; (vi) a reduction in the number of tumor-associated macrophages (TAM) compared to just prior to initiation of administration of the agent; and/or (vii) a decrease in CD14dim/CD16+ nonclassical monocytes in peripheral blood compared to just prior to initiation of administration of the agent.

In some of any such embodiments, the serum enzyme is selected from alanine aminotransferase (ALT), AST, creatine kinase (CK) and LDH. In some of any such embodiments, the serum cytokine is selected from nitric oxide synthase (iNOS), prostaglandin E(2) (PGE(2)), IL-6, IL-1β, IL-8, CCL2, CXCL10, TNF-α, CCL7, CXCL5, CXCL9, CXCL6, MMP-7, MMP-2, and MMP-9. In some of any such embodiments, the reduction or increase is by greater than or greater than about 1.2-fold, 1.5-fold, 2-fold, 3-fold, 4-fold, 5-fold, 10-fold or more.

In some of any such embodiments, the toxic outcome or symptom in the subject is reduced or ameliorated compared to a method in which the therapeutic agent is administered to the subject in the absence of the agent. In some cases, the toxic outcome or symptom is associated with neurotoxicity or cytokine release syndrome (CRS), which optionally is severe neurotoxicity or severe CRS. In some of any such embodiments, the toxic outcome or symptom in the subject at up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent is not detectable or is reduced as compared to a method in which the therapeutic agent is administered to the subject in the absence of the agent. In some embodiments, the toxic outcome or symptom is reduced by greater than or greater than about 1.2-fold, 1.5-fold, 2-fold, 3-fold, 4-fold, 5-fold, 10-fold or more.

In some of any such embodiments, the toxic outcome or symptom is associated with neurotoxicity. In some cases, the neurotoxicity is severe neurotoxicity and/or the neurotoxicity is a grade 3 or higher neurotoxicity. In some embodiments, the toxic outcome or symptom is associated with grade 3, grade 4 or grade 5 neurotoxicity.

In some of any such embodiments, the toxic outcome or symptoms is one or more of confusion, delirium, expressive aphasia, obtundation, myoclonus, lethargy, altered mental status, convulsions, seizure-like activity, seizures (optionally as confirmed by electroencephalogram [EEG]), cerebral edema, elevated levels of beta amyloid (Aβ), elevated levels of glutamate, and elevated levels of oxygen radicals, encephalopathy, dysphasia, tremor, choreoathetosis, symptoms that limit self-care, symptoms of peripheral motor neuropathy, symptoms of peripheral sensory neuropathy and combinations thereof.

In some of any such embodiments, the toxic outcome or symptom of neurotoxicity in the subject at day up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent is not detectable or is reduced as compared to a method in which the therapeutic agent is administered to the subject in the absence of the agent. In some cases, the toxic outcome or symptom of neurotoxicity is reduced by greater than or greater than about 1.2-fold, 1.5-fold, 2-fold, 3-fold, 4-fold, 5-fold, 10-fold or more.

In some of any such embodiments, the method is such that: (i) the administration of the therapeutic agent does not induce neurotoxicity in the subject or does not induce severe neurotoxicity in the subject; (ii) the administration of the therapeutic agent does not induce grade 3 or higher neurotoxicity in the subject, does not induce grade 2 or higher neurotoxicity in the subject or does not induce grade 1 or higher neurotoxicity in the subject; (iii) based on clinical data, administration of the therapeutic agent does not induce neurotoxcity or does not induce severe neurotoxicity in a majority of subjects so treated; or (iv) based on clinical data, administration of the therapeutic agent does not result in a toxic outcome or symptom of neurotoxicity greater than grade 3, greater than grade 2 or greater than grade 1 in a majority of the subjects to treated.

In some of any such embodiments, the toxic outcome or symptom is cerebral edema or is associated with cerebral edema. In some instances, the method is such that the administration of the therapeutic agent does not induce cerebral edema in the subject or based on clinical data, a majority of subjects so treated do not exhibit a cerebral edema after the administration of the therapy.

In some of any such embodiments, the toxic outcome or symptom is associated with cytokine-release syndrome (CRS). In some cases, the CRS is severe CRS and/or the CRS is grade 3 or higher CRS. In some embodiments, the toxic outcome or symptom is associated with grade 3, grade 4 or grade 5 CRS.

In some of any such embodiments, the toxic outcome or symptom is one or more of persistent fever, hypotension, hypoxia, neurologic disturbances, or elevated serum level of an inflammatory cytokine or C reactive protein (CRP).

In some of any such embodiments, the toxic outcome or symptom of CRS in the subject at day up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent is not detectable or is reduced as compared to a method in which the therapeutic agent is administered to the subject in the absence of the agent. In some cases, the CRS is reduced by greater than or greater than about 1.2-fold, 1.5-fold, 2-fold, 3-fold, 4-fold, 5-fold, 10-fold or more.

In some of any such embodiments, the method is such that (i) the administration of the therapeutic agent does not induce CRS in the subject or does not induce severe CRS in the subject; (ii) the administration of the therapeutic agent does not induce grade 3 or higher CRS in the subject, does not induce grade 2 or higher CRS in the subject or does not induce grade 1 or higher CRS in the subject; (iii) based on clinical data, administration of the therapeutic agent does not induce CRS or does not induce severe CRS in a majority of subjects so treated; or (iv) based on clinical data, administration of the therapeutic agent does not result in a toxic outcome or symptom of CRS greater than grade 3, greater than grade 2 or greater than grade 1 in a majority of the subjects to treated.

In some of any such embodiments, the disease or condition is a tumor or a cancer. In some of any such embodiments, the disease or condition is a leukemia or lymphoma. In some of any such embodiments, the disease or condition is a non-Hodgkin lymphoma (NHL), an acute lymphoblastic leukemia (ALL) or a chronic lymphocytic leukemia (CLL).

In some of any such embodiments, the recombinant receptor binds to, recognizes or targets an antigen associated with a disease or condition. In some of any such embodiments, the recombinant receptor is a T cell receptor or a functional non-T cell receptor.

In some of any such embodiments, the recombinant receptor is a chimeric antigen receptor (CAR). In some instances, the CAR contains an extracellular antigen-recognition domain that specifically binds to the antigen and an intracellular signaling domain containing an ITAM. In some of any such embodiments, the antigen is CD19. In some of any such embodiments, the intracellular signaling domain contains an intracellular domain of a CD3-zeta (CD3ζ) chain.

In some of any such embodiments, the CAR further contains a costimulatory signaling region. In some examples, the costimulatory signaling domain contains a signaling domain of CD28 or 4-1BB.

In some of any such embodiments, the cells of the cell therapy are CD4+ or CD8+ T cells. In some of any such embodiments, the cells of the cell therapy are autologous to the subject. In some of any such embodiments, the cells are allogeneic to the subject.

In some of any such embodiments, the therapeutic agent is administered in a sufficient dose, without the administration of the agent, to reduce burden of the disease or condition in the subject as indicated by one or more factors indicative of disease burden, wherein the disease burden optionally is a tumor burden. In some aspects, the reduction in burden includes a reduction in total number of cells of the disease in the subject, in an organ of the subject, in a tissue of the subject, or in a bodily fluid of the subject, a reduction in mass or volume of a tumor, and/or a reduction in number and/or extent of metastases.

In some of any such embodiments, the dose of cells is sufficient, without administration of the agent, to result in partial remission or complete remission in a majority of subjects so treated with the dose of cells; or the disease or condition is a cancer and the dose of cells is sufficient, without administration of the agent, to reduce burden of disease from morphological disease to detectable molecular disease and/or minimum residual disease in a majority of subjects so treated; and/or the disease is a leukemia or lymphoma and the dose of cells is sufficient, without administration of the agent, to reduce the blast cells in the bone marrow to less than or about less than 5%.

In some of any such embodiments, the cell therapy is administered in a sufficient dose, without the administration of the agent, such that: there is a maximum concentration or number of cells of the cell therapy in the blood of the subject of at least at or about 10 cells of the cell therapy per microliter, at least 50% of the total number of peripheral blood mononuclear cells (PBMCs), at least at least about 1×10⁵ cells of the cell therapy, or at least 5,000 copies of recombinant receptor-encoding DNA per micrograms DNA; and/or at day 90 following the initiation of the administration, cells of the cell therapy are detectable in the blood or serum of the subject; and/or at day 90 following the initiation of the administration, the blood of the subject contains at least 20% cells of the cell therapy, at least 10 cells of the cell therapy per microliter or at least 1×10⁴ recombinant receptor-expressing cells.

In some of any such embodiments, the cell therapy includes administration of a dose containing a number of cells between or between about 0.5×10⁶ cells/kg body weight of the subject and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells·kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some of any such embodiments, the dose of cells is a dose that, when administered in the absence of the agent, does, or is likely to, result in severe CRS or grade 3 or higher CRS in the majority of subjects so treated; or the dose of cells is a dose that, when administered in the absence of the agent, does, or is likely to, result in severe neurotoxicity or grade 3 or higher neurotoxicity in the majority of subjects so treated.

In some of any such embodiments, the cell therapy is administered at a dose that is higher than a method in which the cell therapy is administered without administering the agent, whereby the agent ameliorates the risk of a toxic outcome to the cell therapy that would occur, or would likely occur, if a similar dose of the cell therapy is administered in the absence of the agent. In some instances, the dose is at least 1.5-fold, 2-fold, 3-fold, 4-fold, 5-fold or 10-fold greater.

In some of any such embodiments, the cell therapy includes administration of a dose containing a number of cells between about 2×10⁶ cells per kilogram (cells/kg) body weight and about 6×10⁶ cells/kg, between about 2.5×10⁶ cells/kg and about 5.0×10⁶ cells/kg, or between about 3.0×10⁶ cells/kg and about 4.0×10⁶ cells/kg, each inclusive; between about 1.5×10⁸ cells and 4.5×10⁸ cells, between about 1.5×10⁸ cells and 3.5×10⁸ cells or between about 2×10⁸ cells and 3×10⁸ cells, each inclusive; or between about 1.5×10⁸ cells/m² and 4.5×10⁸ cells/m², between about 1.5×10⁸ cells/m² and 3.5×10⁸ cells/m² or between about 2×10⁸ cells/m² and 3×10⁸ cells/m², each inclusive.

In some of any such embodiments, the cell therapy is administered as a single pharmaceutical composition containing the cells. In some of any such embodiments, the cell therapy contains a dose of cell that is a split dose, wherein the cells of the dose are administered in a plurality of compositions, collectively containing the cells of the dose, over a period of no more than three days.

In some of any such embodiments, the agent is administered, or each administration of the agent is independently administered, in a dosage amount of from or from about 0.2 mg per kg body weight of the subject (mg/kg) to 200 mg/kg, 0.2 mg/kg to 100 mg/kg, 0.2 mg/kg to 50 mg/kg, 0.2 mg/kg to 10 mg/kg, 0.2 mg/kg to 1.0 mg/kg, 1.0 mg/kg to 200 mg/kg, 1.0 mg/kg to 100 mg/kg, 1.0 mg/kg to 50 mg/kg, 1.0 mg/kg to 10 mg/kg, 10 mg/kg to 200 mg/kg, 10 mg/kg to 100 mg/kg, 10 mg/kg to 50 mg/kg, 50 mg/kg to 200 mg/kg, 50 mg/kg to 100 mg/kg or 100 mg/kg to 200 mg/kg; or the agent is administered, or each administration of the agent is independently administered, in a dosage amount of from or from about 25 mg to 2000 mg, 25 mg to 1000 mg, 25 mg to 500 mg, 25 mg to 200 mg, 25 mg to 100 mg, 25 mg to 50 mg, 50 mg to 2000 mg, 50 mg to 1000 mg, 50 mg to 500 mg, 50 mg to 200 mg, 50 mg to 100 mg, 100 mg to 2000 mg, 100 mg to 1000 mg, 100 mg to 500 mg, 100 mg to 200 mg, 200 mg to 2000 mg, 200 mg to 1000 mg, 200 mg to 500 mg, 500 mg to 2000 mg, 500 mg to 1000 mg or 1000 mg to 2000 mg, each inclusive.

In some embodiments, the agent is administered, or each administration of the agent is independently administered, in a dosage amount of at least or at least about or about 0.2 mg per kg body weight of the subject (mg/kg), 1 mg/kg, 3 mg/kg, 6 mg/kg, 10 mg/kg, 20 mg/kg, 30 mg/kg, 50 mg/kg, 100 mg/kg or 200 mg/kg; or the agent is administered, or each administration of the inhibitor is independently administered, in a dosage amount of at least or at least about 25 mg, 50 mg, 100 mg, 200 mg, 400 mg, 500 mg, 600 mg, 800 mg, 1000 mg, 1200 mg, 1600 mg or 2000 mg.

In some of any such embodiments, the agent is administered daily, every other day, once a week or once a month. In some of any such embodiments, the agent is administered daily in a dosage amount of at least or at least about 25 mg/day, 50 mg/day, 100 mg/day, 200 mg/day, 400 mg/day, 500 mg/day, 600 mg/day, 800 mg/day, 1000 mg/day, 1200 mg/day, 1600 mg/day or 2000 mg/day.

In some of any such embodiments, the inhibitor is administered orally, subcutaneous or intravenously.

In some of any such embodiments, the subject is a human subject.

Also provided herein is a combination containing a first composition containing genetically engineered cells expressing a recombinant receptor that specifically binds to an antigen and a second composition containing an inhibitor of colony stimulating factor 1 receptor (CSF1R). In some embodiments, the inhibitor reduces the expression of a microglial activation marker on microglial cells, reduces the level or amount one or more effector molecule associated with microglial cell activation in a biological sample; alters microglial cell homeostasis; decreases or blocks microglial cell proliferation; and/or reduces or eliminates microglial cells.

In some of any such embodiments, the inhibition of CSF-1R and/or the reduction of microglial cell activation by the agent is transient and/or is reversible upon discontinued administration of the agent. In some of any such embodiments, the inhibitor is a small molecule, peptide, protein, antibody or antigen-binding fragment thereof, an antibody mimetic, an aptamer, or a nucleic acid molecule.

In some of any such embodiments, the inhibitor is selected from PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof; emactuzumab, IMC-CS4, FPA008, LY-3022855, AMG-820 and TG-3003; or a combination of any of the foregoing. In some examples, the inhibitor is PLX-3397.

In some of any such embodiments, the recombinant receptor binds to, recognizes or targets an antigen associated with a disease or condition. In some of any such embodiments, the recombinant receptor is a T cell receptor or a functional non-T cell receptor.

In some of any such embodiments, the recombinant receptor is a chimeric antigen receptor (CAR). In some aspects, the CAR contains an extracellular antigen-recognition domain that specifically binds to the antigen and an intracellular signaling domain containing an ITAM. In some of any such embodiments, the antigen is CD19. In some cases, the intracellular signaling domain contains an intracellular domain of a CD3-zeta (CD3ζ) chain.

In some of any such embodiments, the CAR further contains a costimulatory signaling region. In some examples, the costimulatory signaling domain contains a signaling domain of CD28 or 4-1BB.

In some of any such embodiments, the genetically engineered cells include T cells or NK cells. In some of any such embodiments, the cells contain T cells that are CD4+ or CD8+ T cells. In some of any such embodiments, the cells are primary cells obtained from a subject, optionally a human subject.

In some of any such embodiments, the cells are formulated for single dosage administration or multiple dosage administration, which optionally contains a split dose of cells. In some embodiments, the inhibitor is formulated for single dosage administration or multiple dose administration.

In some of any such embodiments, provided is a kit containing the combination as described herein and, optionally, instructions for administering the compositions to a subject for treating a disease or condition. In some cases, the disease or condition is a tumor, optionally a cancer.

Also provided is an article of manufacture containing a pharmaceutical composition containing engineered immune cells and/or a T cell-engaging therapy and instructions for administration of the composition to a subject having a disease or condition, in combination with an agent capable of reducing or preventing or blocking activation or function of microglial cells in the subject.

Also provided is an article of manufacture containing a pharmaceutical composition containing an agent capable of reducing or preventing or blocking activation or function of microglial cells; and instructions for administration of the composition to a subject having a disease or condition, in combination with an agent for treating said disease or condition, which agent contains an engineered immune cell and/or T cell-engaging therapy. In some of any such embodiments, the disease or condition is a tumor, optionally a cancer. In some embodiments, the instructions specify the additional therapeutic agent or therapy is for administration prior to, with or at the same time and/or subsequent to initiation of administration of the engineered immune cell and/or T cell-engaging therapy. In some of any such embodiments, the instructions further specify the engineered immune cell and/or T cell-engaging therapy is for parenteral administration, optionally intravenous administration.

In some of any such embodiments, the engineered immune cell and/or T cell-engaging therapy comprises primary T cells obtained from a subject. In some aspects, the T cells are autologous to the subject. In some embodiments, the T cells are allogeneic to the subject.

In some of any such embodiments, the kit or article of manufacture comprises one of a plurality of compositions of the cell therapy comprising a first composition of genetically engineered cells comprising CD4+ T cells or CD8+ T cells, wherein the instructions specify the first composition is for use in with a second composition comprising the other of the CD4+ T cells or the CD8+ T cells, optionally wherein the cells of the first composition and cells of the same composition are from the same subject.

In some of any such embodiments, the agent is a small molecule, peptide, protein, antibody or antigen-binding fragment thereof, an antibody mimetic, an aptamer, or a nucleic acid molecule. In some aspects, the agent is selected from: PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof; emactuzumab, IMC-CS4, FPA008, LY-3022855, AMG-820 and TG-3003; or a combination of any of the foregoing. In some cases, the agent is PLX-3397.

### BRIEF DESCRIPTION OF THE DRAWINGS

FIG. 1 is a Kaplan meier curve depicting observed time to onset of CRS and neurotoxicity.

FIGS. 2A and 2B shows the duration of response (CR/PR, CR or PR) and overall survival in the full and core cohort of subjects.

FIG. 3 shows a graph plotting progression-free time (months) and indicating best overall response and response durability, and individual clinical outcomes observed over time in individual subjects within a Full cohort and a Core cohort of NHL subjects treated with an anti-CD19 cell therapy containing CAR-T-expressing CD4+ and CD8+ T cells.

### DETAILED DESCRIPTION

Provided herein are methods, compositions, and combinations for ameliorating a therapy-induced toxicity, such as in connection with the administration of an immunotherapy or immunotherapeutic agent. Among the a composition including cells for adoptive cell therapy, e.g., such as a T cell therapy (e.g. T cells expressing a recombinant receptor such as CAR-expressing T cells) or a T cell-engaging therapeutic agent, such as a bispecific or other multispecific agent, e.g., antibody that is capable of recruiting and/or engaging the activity of one or more T cells, such as in a target-specific manner. In some aspects, the provided embodiments involve the administration of such therapeutic agents in combination or connection with an agent to reduce the risk of, prevent or ameliorate toxicity; for example, provided are combination therapies for effecting such administration. In some embodiments, the combination therapy or method involves administration of an agent, such as an anti-inflammatory or anti-oxidant agent or agent that reduces, prevents, impairs and/or ablates microglial cell activity, or is capable of doing so, such as an inhibitor of microglial cell activity, e.g., a CSF1R inhibitor, and administration of the immunotherapy or immunotherapeutic agent, such as a composition including cells for adoptive cell therapy, e.g., such as a T cell therapy (e.g., CAR-expressing T cells), a cell therapy involving the induction of an immune response, directly or indirectly, and/or a T cell-engaging therapeutic agent.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section heading used herein are for organizational purposes only and are not to be construed as limiting the subject matter described.

### I. Overview

Provided herein are methods of ameliorating or reducing or preventing or reducing risk of a toxicity, such as an actual, e.g., developing, or potential therapy-induced toxicity. In some embodiments, the methods involve administrating a combination therapy (e.g., administering simultaneously or sequentially) of a cell-based therapy, such as an adoptive cell therapy and an additional agent. Among such agents are agents that modulate a component of an anti-inflammatory or anti-oxidant or oxidative response or oxidative stress pathway, such as an anti-inflammatory agent or anti-oxidant agent, and agents that reduce, impair, prevent or ablate microglial cell activity and/or a parameter or factor associated therewith. In some aspects, the agent is an activator or promoter of NRF2 or an NRF2-related pathway, a modulator or modifier of KEAP1, or an agent that promotes activation of expression via an antioxidant response element (ARE). In some embodiments, the cell therapy is or comprises a tumor infiltrating lymphocytic (TIL) therapy, a transgenic TCR therapy or a recombinant-receptor expressing cell therapy (optionally T cell therapy), which optionally is a chimeric antigen receptor (CAR)-expressing cell therapy. Thus, in some embodiments, the immunotherapy involves the administration of a composition containing a plurality of tumor-infiltrating lymphocytes (TILs), a plurality of cells, such as T cells, e.g., engineered T cells, expressing a recombinant receptor, such as a TCR or a chimeric antigen receptor In some embodiments, the recombinant receptor is a TCR. In some cases, the recombinant receptor is a chimeric antigen receptor (CAR).

In some embodiments, the additional agent, such as the agent administered to prevent or reduce or ablate one or more properties associated with microglial activity or function, alters microglial homeostasis and/or viability, induces a decrease or blockade or prevention of microglial cell proliferation, causes a reduction or elimination of microglial cells, or causes a reduction or prevention in or of microglial activation. In some embodiments, such agent, e.g., that reduces microglial cell activity or associated property, targets, and optionally is an inhibitor and/or blocker of, colony stimulating factor 1 receptor (CSF1R). In some embodiments, the inhibitor is or comprises PLX3397. In some embodiments, the method further involves administering a lymphodepleting therapy and/or the composition or combination further comprises a lymphodepleting agent, such as a lymphodepleting chemotherapeutic agent.

In some embodiments, the agent is an agent that activates or promotes the activation or upregulation of the transcription factor NRF2 (also called nuclear factor (erythroid-derived 2)-like 2, or NFE2L2) and/or of an NRF2-regulated or NRF2-related pathway; an agent that activates, promoters or upregulates expression of one or more genes having or capable of being activated by an antioxidant response element (ARE); an agent that activates or promotes phase II detoxicfication, anti-oxidant enzymes or anti-inflammatory or antioxidant activities thereof; an agent that promotes anti-oxidant or anti-inflammatory pathways; or an agent that results in the kelch-like ECH-associated protein 1 (KEAP1).

Adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in treating cancer and other diseases and disorders. In certain contexts, available approaches to adoptive cell therapy may not always be entirely satisfactory. In some contexts, one or more desired outcomes, such as optimal efficacy, can depend on the ability of the administered cells (e.g., the ability of one or more subpopulations thereof) to carry out one or more activities or functions and/or to exhibit one or more particular properties. In some aspects, optimal efficacy depends upon the cells' ability to recognize and bind to a target, e.g., target antigen; in some aspects, it depends upon the ability of the cells to traffic, localize to and/or successfully enter and/or circulate through one or more appropriate sites within the subject, such as sites or tissues expressing target antigen or in which activity is desired. Exemplary of sites for entry are tumors, and environments thereof, e.g., microenvironments, vasculature, and/or lymphoid system or organs. Optimal efficacy typically depends upon the ability of the cells to become activated, expand, and/or to exert various effector functions, including cytotoxic killing and/or secretion of various factors such as cytokines. Optimal efficacy may depend upon the ability of the engineered cells to persist in desired locations or environments and/or for desired periods of time, such as long-term and/or within tumor or disease environments. In some aspects, optimal efficacy may depend upon at least a subset of the cells' ability to differentiate, transition or engage in reprogramming into one or more certain phenotypic states (such as effector, long-lived memory, less-differentiated, and effector states). In some embodiments, optimal efficacy may depend upon the cells' ability to effect recall responses, such as robust and effective recall responses, in contexts following clearance and re-exposure to target ligand or antigen, such as following clearance of disease (such as reexposure to antigen, such as in the context of relapse, in a subject having previously achieved complete remission, optionally minimal residual disease (MRD) negative remission); thus, in some aspects, optimal efficacy may depend on the ability of cells to and avoid adopting a less-optimal state or phenotype following initial or early exposure to antigen, such as the ability of the cells to avoid becoming exhausted or anergic or terminally differentiated (or to exhibit reduced degrees of exhaustion anergy terminal differentiation compared to a reference cell population); in some aspect, optimal efficacy may depend upon the cells' ability to avoid adopting or differentiating into a suppressive state.

In some aspects, the provided embodiments are based on observations that the efficacy of adoptive cell therapy may be limited in some context by the development of, or risk of developing, toxicity or one or more toxic outcomes in the subject to whom such cells are administered. In some cases, such toxicities can be severe. For example, in some cases, administering a dose of cells expressing a recombinant receptor, e.g., a CAR, can result in toxicity or risk thereof, such as CRS or neurotoxicity. In some cases, risk of one or more toxic outcomes may increase in a manner correlated with increases of properties associated with improved efficacy. For example, while in some contexts the administration of relatively higher doses of such cells can increase efficacy, for example, by increasing exposure to the cells such as by promoting expansion and/or persistence, they may also result in an even greater risk of developing a toxicity or a more severe toxicity. Similarly, while the co-administration of one or more agents to promote immune function, may in some contexts promote desired activity and function such as secretion of cytokines and target-specific cytotoxicity, and/or reduce suppressive factors, it may in certain aspects also be associated with an increased risk of one or more factors associated with toxicity. In some cases, the administration of a lymphodepleting therapy, such as a high intensity lymphodepleting therapy, administered prior to the administration of the cell therapy may increase the efficacy of the treatment. In some aspects, however, such preconditioning may also result in a greater risk of developing a toxicity or toxic outcome, e.g., a severe toxicity or sign or symptom thereof. In some cases, subjects having higher disease burden may be at a greater risk for developing a toxicity or outcome thereof a more severe form of toxicity.

Certain available methods for treating or ameliorating toxicity may not always be entirely satisfactory. In some contexts, available methods for treating or ameliorating toxicity are limited or hindered by a lack of understanding in the cause of toxicity. For example, it has not been entirely understood how some therapies and/or particular cell therapy or therapies may cause or be at risk for leading to toxicity, such as CRS, neurotoxicity, and/or cerebral edema.

Many available approaches focus, for example, on targeting downstream effects of toxicity, such as by cytokine blockade, and/or delivering agents such as high-dose steroids which can also eliminate or impair the function of administered cells. Additionally, such approaches often involve administration of such interventions only upon detection of physical signs or symptoms of toxicity and/or certain degrees or levels thereof, which in general involve signs or symptoms of moderate or severe toxicity (e.g. moderate or severe CRS), which in many cases may be associated with risk of inefficacy of the intervention and/or require administration of greater dosage or higher intensity intervention, which may be associated with one or more undesirable side effects and/or reduce efficacy of the therapy. In some embodiments, available approaches are not entirely satisfactory in their ability to reduce or prevent one or more of various forms of toxicity such as neurotoxicity.

In some cases, available agents and/or therapies aimed at reducing or ameliorating therapy-associated toxicity (e.g., steroids) are themselves associated with toxic side effects. The intensity of such side effects may be greater at higher dosages of the agents and/or therapies, such as at the relatively higher dose or frequency that may be required in order to treat or ameliorate the severity of the toxicity at the time administered, e.g., after the sign or symptom or level or degree thereof. In addition, in some aspects, the available agent or therapy for treating a toxicity may limit the efficacy of the cell therapy, such as the efficacy of the chimeric receptor (e.g. CAR) expressing cells provided as part of the cell therapy (Sentman, Immunotherapy, 5:10 (2013)), e.g., by reducing activity or one or more desired downstream effects induced by such therapy.

In some embodiments, there may be communications, such as bi-directional communications, between the immune system, such as via inflammatory factors and/or cytokines, and the brain. In some cases, challenges to the immune system can be sensed by the nervous system, such as using neural and/or humoral pathways. In some aspects, there may be communications via the neurovascular unit (e.g., endothelium), brainstem, blood brain barrier, or circumventricular organs of the brain (See e.g. Erickson et al., Neuroimmunomodulation. (2012) 19(2):121-130). In some instances, communications between the immune system and brain may lead to microglial propagation of cytokines or chemokines in the brain (DiSabato et al., J Neurochem. (2016) 139 Suppl 2:136-153). In some contexts, microglia may respond in an adaptive manner to potential threats to central nervous system homeostasis. Microglia activation may, in some aspects, be neurotoxic (Hanisch et al., Nat Neurosci. (2007) 10(11):1387-1394; Koyabashi et al., Cell Death Dis. (2013) 7; 4:e525).

Provided are embodiments involving modulating, such as preventing or reducing, pro-inflammatory cytokines or stress cytokines, activating or promoting an anti-oxidant response, promoting a neuroprotective phenotype and/or blocking or reducing microglial cell activation. Provided are compositions, combinations and methods offering advantages over available approaches for ameliorating or reducing toxicity. In some embodiments, the method or composition or combination reduces or ameliorates potential toxicities that may be associated with certain therapies when administered to a subject. In some embodiments, the methods, compositions and combinations involve and/or are useful in the administration of, e.g., in a combination therapy, a cell based therapy and another agent such as an agent that reduces microglial cell activity or an anti-inflammatory or anti-oxidative stress agent.

In some embodiments, the cell therapy is a tumor infiltrating lymphocytic (TIL) therapy, a transgenic TCR therapy or a recombinant-receptor expressing cell therapy (optionally T cell therapy), which optionally is a chimeric antigen receptor (CAR)-expressing cell therapy. In some embodiments, the recombinant receptor is a TCR. In some cases, the recombinant receptor is a chimeric antigen receptor (CAR). In some embodiments, the method further involves administering a lymphodepleting therapy.

In some embodiments, the provided method ameliorates toxicity that is associated with the activation of microglial cells during the administration of a cell therapy. In some cases, cells administered for adoptive cell therapy may prompt the production of cytokines in the body. In some cases, cytokine produced in the body can enter the brain and cause adverse outcomes and/or cells in the brain (e.g., cells of the therapy and/or cells activated thereby, directly or indirectly) are induced to produce such cytokines, which in turn cause adverse outcomes. In some cases, increased levels of cytokines or other factors in the brain can activate microglia and can cause adverse effects such as toxicity or one or more outcomes or aspects thereof. Thus, in some cases, microglia play a role in therapy-induced toxicity and the provided method involves administration of an agent that reduces microglial cell activity, e.g. an inhibitor that targets microglia activity. In some embodiments, the method involves administering an inhibitor of CSF1R signaling (also known as CD115, c-fms, CFMS, Colony Stimulating Factor 1 Receptor, Colony Stimulating Factor I Receptor, CSF 1 R, CSF-1-R, CSF1R, CSFR, FIM2, FMS Proto-oncogene, Macrophage Colony Stimulating Factor I Receptor, MCSF Receptor), which is important for microglial cell migration, differentiation, and survival. Therefore, in some embodiments, the methods provided herein reduce or eliminate the toxic outcomes that may be directly or indirectly caused by microglial cells.

In some aspects, the toxic outcomes may include but are not limited to symptoms of cytokine release syndrome, neurotoxicity, and/or cerebral edema. In some cases, cerebral edema co-presents with or is a feature of neurotoxicity. Thus, in certain aspects, the provided methods and other embodiments provide advantages over an approach which focuses, for example, on targeting certain downstream effects of toxicity, such as by cytokine blockade, and/or that involve delivering agents such as high-dose steroids which can also eliminate or impair the function of administered cells.

In some embodiments, the method involves administering a cell therapy to a subject having a disease or condition, wherein the cell therapy contains cells that secrete an agent that reduces microglial cell activity, such as an inhibitor of microglial cell activation and/or an inhibitor colony-stimulating factor-1 receptor (CSF1R). Thus, in some embodiments, toxic side effects that are associated with certain agents and therapies (e.g., steroids) available for use in ameliorating toxicity themselves are avoided. In some embodiments, the use of a microglia inhibitor to ameliorate toxicity does not impact or does not substantially impact the efficacy of or does not reduce or does not substantially reduce the efficacy of the cell therapy and/or one or more particular activities, functions or properties thereof, such as does not impact or reduce (or substantially impact or reduce) persistence, expansion, cytokine secretion by, and/or activation of, cells of the cell therapy, e.g., for a specified time period or at a particular time point, following administration in vivo and/or in an in vitro assay correlative with efficacy.

In some embodiments, the method involves administering the cell therapy comprising a recombinant receptor to a subject that has been previously administered a therapeutically effective amount of an agent that reduces or is capable of preventing or reducing or blocking microglial cell activity, such as an inhibitor of microglia activity, e.g., a CSF1R inhibitor; and/or provided are combinations or compositions for such administration, such as kits comprising one or more such agents, and instructions for administration in combination with the other agent in accordance with such methods. In some embodiments, the provided methods involve administering the agent that reduces microglial cell activity to a subject before administering a dose of cells expressing a recombinant receptor to the subject; and/or provided are combinations or compositions for such administration, such as kits comprising one or more such agents, and instructions for administration in combination with the other agent in accordance with such methods. In some embodiments, the treatment with the agent occurs at a time at which no physical signs or symptoms of neurotoxicity have developed. Thus, in some cases, the methods (or compositions and/or combinations) provide an ability to intervene before undesired toxicity-related outcomes can result and does not rely upon the detection of symptoms, especially symptoms of severe toxicity.

In some embodiments, the agent that reduces microglial cell activity is not further administered after initiation of the cell therapy. In other embodiments, the method involves administering the agent after administration of the cell therapy. In some embodiments, the method involves administering the inhibitor prior to and after initiation of the cell therapy. Thus, in some embodiments, the administration of the agent results in transient inhibition of activity of the microglia and/or CSF1R. Therefore, in some aspects, the inhibition of microglia and/or CSF1R activity is not long lasting or permanent.

In some embodiments, the subject is at risk for having a therapy-induced adverse symptom. For example, the therapy-induced adverse symptom is associated with neurotoxicity or cytokine release syndrome or is cerebral edema. In some cases, cerebral edema co-presents with neurotoxicity. In some cases, a toxic outcome or symptom in the subject is reduced or ameliorated compared to a method in which the cell therapy is administered to the subject in the absence of the inhibitor. For example, in some embodiments, the toxic outcome or symptom is associated with neurotoxicity or cytokine release syndrome (CRS), which optionally is severe neurotoxicity or severe CRS. In some embodiments, the agent, e.g. inhibitor, is administered to a subject after exhibiting a clinical sign or symptom of a toxicity-related outcome. For example, the symptom of the toxicity-related outcome includes fever, hypertension, hypoxia, neurologic disturbances, or a serum level of an inflammatory cytokine or C reactive protein (CRP). In some aspects, the toxicity-related outcome is associated with neurotoxicity such as confusion, delirium, expressive aphasia, obtundation, myoclonus, lethargy, altered mental status, convulsions, seizure-like activity, seizures (optionally as confirmed by electroencephalogram), elevated levels of beta amyloid, elevated levels of glutamate, and elevated levels of oxygen radicals. In some cases, the methods reduce toxic outcome or the potential of a toxic outcome, allowing an increased dosage of cells expressing a recombinant receptor to be administered.

In some embodiments, the cell therapy is a tumor infiltrating lymphocytic (TIL) therapy, a transgenic TCR therapy or a recombinant-receptor expressing cell therapy (optionally T cell therapy). For example, in some embodiments, the cell therapy is an adoptive cell therapy, including a therapy involving administration of cells expressing chimeric receptors specific for a disease or disorder of interest, such as chimeric antigen receptors (CARs) and/or other recombinant antigen receptors, as well as other adoptive immune cells and adoptive T cell therapies. In some embodiments, the adoptive cell therapy includes administration of a dose of cells expressing a recombinant receptor, such as a CAR or other recombinant antigen receptor. In some embodiments, the therapy targets CD19 or is a B cell targeted therapy. In some embodiments, the method involves administering a cell therapy to a subject having a disease or condition, wherein the cell therapy contains cells that are further engineered to secrete an inhibitor of microglia. In some embodiments, the method involves administering a cell therapy to a subject having a disease or condition, wherein the cell therapy contains cells that are further engineered to secrete an inhibitor of microglia activity. In some embodiments, the cells are engineered to secrete an inhibitor of colony-stimulating factor-1 receptor (CSF1R).

In some embodiments, the agent that reduces microglial cell activation is selected from an anti-inflammatory agent, an inhibitor of NADPH oxidase (NOX2), a calcium channel blocker, a sodium channel blocker, inhibits GM-CSF, inhibits CSF1R, specifically binds CSF-1, specifically binds IL-34, inhibits the activation of nuclear factor kappa B (NF-κB), activates a CB₂ receptor and/or is a CB₂ agonist, a phosphodiesterase inhibitor, inhibits microRNA-155 (miR-155) or upregulates microRNA-124 (miR-124).

In some embodiments, the agent that reduces microglial cell activity is an inhibitor of CSF1 or CSF1R. For example the inhibitor is selected from MCS110, PLX-3397, PLX7486, JNJ-40346527, JNJ28312141, ARRY-382, PLX73086 (AC-708), DCC-3014, AZD6495, GW2580, Ki20227, BLZ945, PLX647, PLX5622, emactuzumab (RG7155; R05509554), Cabiralizumab (FPA-008), LY-3022855 (IMC-CS4), AMG-820, TG-3003, MCS110, H27K15, 12-2D6, 2-4A5, Nimodipine, MOR103, IVIg, and LNA-anti-miR-155. In some embodiments, the inhibitor is administered in a form that specifically affects the central nervous system and/or does not affect tumor-associated macrophages.

In some embodiments, the method results in one or more effects such as an alteration in microglial homeostasis, decrease or blockade of microglial cell proliferation, reduction or elimination of microglial cells, reduction in microglial activation, alteration in the level of a serum or blood biomarker of CSF1R inhibition or a decrease in the level of urinary collagen type 1 cross-linked N-telopeptide (NTX) compared to a method wherein the inhibitor is not administered. In some embodiments, the method involves detecting a biomarker indicative of microglial activation and/or CSF1R inhibition.

In some embodiments, administration of the agent decreases tumor burden and/or decreases blast marrow in the subject as compared to a method in which the dose of cells expressing the recombinant receptor is administered to the subject in the absence of the inhibitor. In some embodiments, the dose of cells exhibits increased or prolonged expansion and/or persistence in the subject as compared to a method in which the dose of cells expressing the recombinant receptor is administered to the subject in the absence of the inhibitor.

In some embodiments, the method further involves administering a lymphodepleting therapy prior to the administration of the cell therapy. In some cases, while a higher dose of a lymphodepleting therapy prior to the administration of the cell therapy may increase the efficacy of the treatment, it may also result in an even greater risk of developing a toxicity or a more severe toxicity. Thus, in some cases, the method allows the administration of a higher dose of lymphodepleting agent compared to the dose administered without the inhibitor of microglia activity.

### II. Combination Therapy

Provided herein are methods of ameliorating or reducing a potential therapy-induced toxicity. In some embodiments, the method involves administering to a subject a combination therapy including 1) a therapy associated with a risk of a therapy-induced toxicity, such as an immunotherapy or immunotherapeutic agent, e.g., a cell therapy (e.g., engineered T cell therapy, such as CAR-T cells) and/or T-cell engaging therapy and 2) an agent that reduces microglial cell activity, such as an inhibitor of microglia activity, e.g., a CSF1R inhibitor. In some embodiments, the subject has a disease or condition and the methods are for ameliorating or reducing a potential toxicity associated with treating the disease or condition. In some embodiments, the provided embodiments include methods of treating a subject by administering to a subject a combination therapy including 1) a therapy for treating the disease or condition in which the therapy is associated with a risk of a therapy-induced toxicity, such as is an immunotherapy or is an immunotherapeutic agent, e.g., a cell therapy (e.g., engineered T cell therapy, such as CAR-T cells) and/or T-cell engaging therapy and 2) an inhibitor of microglia activity.

In some embodiments, the method ameliorates or reduces a potential therapy-induced toxicity or adverse symptom. In some embodiments, the therapy is a cell therapy. In some embodiments, the cell therapy is adoptive cell therapy. In some embodiments, the cell therapy is or comprises a tumor infiltrating lymphocytic (TIL) therapy, a transgenic TCR therapy or a recombinant-receptor expressing cell therapy (optionally T cell therapy), which optionally is a chimeric antigen receptor (CAR)-expressing cell therapy. In some embodiments, the therapy targets CD19 or is a B cell targeted therapy. In some embodiments, the cells and dosage regimens for administering the cells can include any as described in the following subsection A under “Administration of Cells.”

In some embodiments, the administration of the cell therapy in the absence of the agent, e.g., inhibitor, is associated with or is capable of inducing a toxic outcome in the subject or in a majority of subjects so treated. For example, in some aspects, the toxic outcome is associated with neurotoxicity or cytokine release syndrome (CRS), which optionally is severe neurotoxicity or severe CRS. In some cases, the toxic outcome is cerebral edema or is associated with cerebral edema.

In some embodiments, the provided methods involve administration of an agent that reduces a microglial cell activity, such as is or comprises a microglia activity inhibitor. In some embodiments, the method results in one or more effects such as an alteration in microglial homeostasis, a decrease or blockade of microglia proliferation, a reduction or elimination of microglial cells, and/or a reduction in microglial activation. In some embodiments, the inhibitor promotes microglia quiescence but does not adversely affect viability of the microglia, eliminate microglia, or reduce the number of microglia. In some embodiments, the inhibitor is a brain specific inhibitor of microglia activity (Ponomarev et al., Nature Medicine, (1):64-70 (2011)).

In some embodiments, the agent that reduces microglial cell activity is a nucleic acid molecule (e.g. siRNA), peptide, polypeptide, small molecule, antibody, or antigen-binding fragment thereof. In some embodiments, the agent reduces microglia activity or microglia proliferation and survival. In some embodiments, the agent that targets CSF1R signaling is selected from a small molecule inhibitor of microglia activity, a monoclonal antibody that inhibits microglia, an inhibitor of macrophage polarization, a CSF1 inhibitor, and a CSF1R inhibitor. In some embodiments, the inhibitor is PLX3397. In some embodiments, the cells and dosage regimens for administering the cells can include any as described in the following subsection B under “Administration of Agents.”

In some embodiments, the administration of the agent reduces the number of microglial cells in the subject by greater than 20%, greater than 30%, greater than 40% or greater than 50%, greater than 60%, greater than 70%, greater than 80%, greater than 90%, greater than 95% or greater than 99%. In some embodiments, the inhibitor is capable of producing an alteration in a level of a serum or blood biomarker of CSF1R inhibition is selected from an increase in plasma CSF-1, an increase in a level of a serum enzyme or a decrease in CD14dim/CD16+ nonclassical monocytes. In some aspects, the serum enzyme is alanine aminotransferase (ALT), AST, creatine kinase (CK) or LDH.

In some embodiments, the agent is administered sequentially, intermittently, or at the same time as or in the same composition as the cell therapy. For example, the agent can be administered prior to, during, simultaneously with, or after administration of the cell therapy. In some embodiments, the method involves administering the cell therapy to a subject that has been previously administered a therapeutically effective amount of the agent that reduces microglial cell activity. In some embodiments, the microglia inhibitor is administered to a subject before administering a dose of cells expressing a recombinant receptor to the subject. In some embodiments, the treatment with the agent occurs at a time at which no physical signs or symptoms of neurotoxicity have developed. In some embodiments, the agent is administered after the administration of the dose of cells.

In some embodiments, the disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g., cancer), autoimmune or inflammatory disease, or an infectious disease, e.g., caused by bacterial, viral or other pathogens. Exemplary antigens, which include antigens associated with various diseases and conditions that can be treated, include any of antigens described herein. In particular embodiments, the engineered cells of the combination therapy express a recombinant receptor, including a chimeric antigen receptor or transgenic TCR, that specifically binds to an antigen associated with the disease or condition.

In some embodiments, the disease or condition is a tumor, such as a solid tumor, lymphoma, leukemia, blood tumor, metastatic tumor, or other cancer or tumor type.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, and parasitic disease, and autoimmune and inflammatory diseases. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma. In some embodiments, the subject has acute-lymphoblastic leukemia (ALL). In some embodiments, the subject has non-Hodgkin's lymphoma.

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is selected from the group consisting of avβ6 integrin (avb6 integrin), Receptor Tyrosine Kinase Like Orphan Receptor 1 (ROR1), B cell maturation antigen (BCMA), tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, type III epidermal growth factor receptor mutation (EGFR vIII), epithelial glycoprotein 2 (EGP-2), EGP-4, epithelial glycoprotein 40 (EPG-40), ephrinB2, ephrine receptor A2 (EPHa2), ErbB2, 3, or 4, erbB dimers, EGFR vIII, estrogen receptor, a folate binding protein (FBP), folate receptor alpha, Fc receptor like 5 FCRL5; also known as Fc receptor homolog 5 or FCRH5), fetal acetylcholine receptor(fetal AchR), ganglioside GD2, O-acetylated GD2 (OGD2), ganglioside GD3, G Protein Coupled Receptor 5D (GPCR5D), HMW-MAA, IL-22R-alpha, IL-13R-alpha2 (IL-13Ra2), kinase insert domain receptor (kdr), kappa light chain, Lewis Y, L1-cell adhesion molecule, (L1-CAM), CE7 epitope of L1-CAM, Leucine Rich Repeat Containing 8 Family Member A (LRRC8A), Melanoma-associated antigen (MAGE)-A1, MAGE-A3, MAGE-A6, Preferentially expressed antigen of melanoma (PRAME), survivin, EGP2, EGP40, TAG72, B7-H6, IL-22 receptor alpha (IL-22R-alpha), IL-13 receptor a2 (IL-13Ra2), carbonic anhydrase 9 (CA9, also known as CAIX or G250), GD3, G Protein Coupled Receptor 5D (GPCR5D), Her2/neu (receptor tyrosine kinase erbB2), Her3 (erb-B3), Her4 (erb-B4), erbB dimers, Human high molecular weight-melanoma-associated antigen (HMW-MAA), CD171, G250/CAIX, hepatitis B surface antigen, Human leukocyte antigen A1 (HLA-AI), MAGE A1, Human leukocyte antigen A2 (HLA-A2), a cancer-testis antigen, cancer/testis antigen 1B (CTAG, also known as NY-ESO-1 and LAGE-2), PSCA, folate receptor-a, CD44v6, CD44v7/8, avb6 integrin, 8H9, neural cell adhesion molecule (NCAM), VEGF receptors, 5T4, Foetal AchR, NKG2D ligands, CD44v6, dual antigen, and an antigen associated with a universal tag, a cancer-testes antigen, MUC1, MUC16, progesterone receptor, a prostate specific antigen, prostate stem cell antigen (PSCA), natural killer group 2 member D (NKG2D) Ligands, melan A (MART-1), glycoprotein 100 (gp100), oncofetal antigen, Trophoblast glycoprotein (TPBG also known as 5T4), tumor-associated glycoprotein 72 (TAG72), vascular endothelial growth factor receptor (VEGFR), vascular endothelial growth factor receptor 2 (VEGF-R2), carcinoembryonic antigen (CEA), oncofetal antigen, prostate specific antigen, prostate specific membrane antigen (PSMA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, C-C Motif Chemokine Ligand 1 (CCL-1), CD138, and a pathogen-specific antigen.

In some embodiments, the antigen recognized or targeted by the recombinant receptor is present on a universal tag, such as a fluorescent tag, e.g., FITC. In some embodiments, the recombinant receptor comprises an antibody or antigen-binding fragment that binds or recognizes an antigen conjugated to FITC, such as a CAR that specifically binds to FITC.

In some embodiments, the methods can be used to treat a myeloma, a lymphoma or a leukemia. In some embodiments, the methods can be used to treat a non-Hodgkin lymphoma (NHL), an acute lymphoblastic leukemia (ALL), a chronic lymphocytic leukemia (CLL), a diffuse large B-cell lymphoma (DLBCL), acute myeloid leukemia (AML), or a myeloma, e.g., a multiple myeloma (MM). In some embodiments, the methods can be used to treat a MM or a DBCBL.

For the prevention or treatment of disease, the appropriate dosage may depend on the type of disease to be treated, the type of inhibitor, the type of cells or recombinant receptors administered in the method, the severity and course of the disease, whether the inhibitor and cells are administered for preventive or therapeutic purposes, previous therapy, the subject's clinical history and response to the agent or the cells, and the discretion of the attending physician. The compositions are in some embodiments suitably administered to the subject at one time or over a series of treatments.

In some embodiments, the immunotherapy, e.g., T cell therapy, and the inhibitor of microglia activity are administered as part of a further combination treatment, which can be administered simultaneously with or sequentially to, in any order, another therapeutic intervention. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the methods further include a lymphodepleting therapy, such as administration of a chemotherapeutic agent. In some embodiments, the methods do not include a lymphodepleting therapy.

Prior to, during or following administration of the immunotherapy (e.g., T cell therapy, such as CAR-T cell therapy) and/or an inhibitor of microglial cell activity, the biological activity of the immunotherapy, e.g., the biological activity of the engineered cell populations, and/or the toxicity of the therapy to the subject in some embodiments is measured, e.g., by any of a number of known methods. Parameters to assess include one or more functions associated with the administered cells and/or one or more toxic outcomes associated with the therapy and/or at risk of developing as a result of the therapy, in which such parameters can be measured using any suitable method known in the art, such as assays described further below in Section IV below. In some embodiments, the biological activity of the cells, e.g., T cells administered for the T cell based therapy, is measured by assaying expression and/or secretion of one or more cytokines. In some aspects the biological activity is measured by assessing the disease burden and/or clinical outcome, such as reduction in tumor burden or load. In some embodiments, one or more toxic outcomes, such as associated with cytokine release syndrome (CRS) or neurotoxicity, are assessed in the subject. In some embodiments, administration of one or both agents of the combination therapy and/or any repeated administration of the therapy, can be determined based on the results of the assays before, during, during the course of or after administration of one or both agents of the combination therapy.

A. Administration of Cells

In some embodiments of the methods, compositions, combinations, kits and uses provided herein, the combination therapy includes administering to a subject an immunotherapy, such as a T cell therapy (e.g. engineered T cell therapy, such as CAR-expressing T cells) or a T cell-engaging therapy. Such therapies can be administered prior to, subsequent to, simultaneously with administration of one or more inhibitors or microglial cell activity as described. In some embodiments, the engineered cells and compositions are administered to a subject or patient having the particular disease or condition to be treated, e.g., via adoptive cell therapy, such as adoptive T cell therapy. In some embodiments, the provided cells and compositions are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell. In some embodiments, the methods ameliorate one or more toxic outcomes associated with the immunotherapy, such as T cell therapy (e.g. engineered T cell therapy, such as CAR-expressing T cells) or a T cell-engaging therapy.

1. T Cell-Engaging Therapy

In some embodiments, the immunotherapy is or comprises a T cell-engaging therapy that is or comprises a binding molecule capable of binding to a surface molecule expressed on a T cell. In some embodiments, the surface molecule is an activating component of a T cell, such as a component of the T cell receptor complex. In some embodiments, the surface molecule is CD3 or is CD2. In some embodiments, the T cell-engaging therapy is or comprises an antibody or antigen-binding fragment. In some embodiments, the T cell-engaging therapy is a bispecific antibody containing at least one antigen-binding domain binding to an activating component of the T cell (e.g. a T cell surface molecule, e.g. CD3 or CD2) and at least one antigen-binding domain binding to a surface antigen on a target cell, such as a surface antigen on a tumor or cancer cell, for example any of the listed antigens as described herein, e.g. CD19. In some embodiments, the simultaneous or near simultaneous binding of such an antibody to both of its targets can result in a temporary interaction between the target cell and T cell, thereby resulting in activation, e.g. cytotoxic activity, of the T cell and subsequent lysis of the target cell.

Among such exemplary bispecific antibody T cell-engagers are bispecific T cell engager (BiTE) molecules, which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010). In some embodiments, the T-cell engaging therapy is blinatumomab or AMG 330. Any of such T cell-engagers can be used in used in the provided methods, compositions or combinations.

2 Cell Therapy

In some embodiments, the immunotherapy is a cell-based therapy that is or comprises administration of cells, such as immune cells, for example T cell or NK cells, that target a molecule expressed on the surface of a lesion, such as a tumor or a cancer. In some embodiments, the immune cells express a T cell receptor (TCR) or other antigen-binding receptor. In some embodiments, the immune cells express a recombinant receptor, such as a transgenic TCR or a chimeric antigen receptor (CAR). In some embodiments, the cells are autologous to the subject. In some embodiments, the cells are allogeneic to the subject. Exemplary of such cell therapies, e.g. T cell therapies, for use in the provided methods are described below.

In some embodiments, the provided cells express and/or are engineered to express receptors, such as recombinant receptors, including those containing ligand-binding domains or binding fragments thereof, and T cell receptors (TCRs) and components thereof, and/or functional non-TCR antigen receptors, such as chimeric antigen receptors (CARs). In some embodiments, the recombinant receptor contains an extracellular ligand-binding domain that specifically binds to an antigen. In some embodiments, the recombinant receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Among the engineered cells, including engineered cells containing recombinant receptors, are described in Section III below. Exemplary recombinant receptors, including CARs and recombinant TCRs, as well as methods for engineering and introducing the receptors into cells, include those described, for example, in international patent application publication numbers WO2000/14257, WO2013/126726, WO2012/129514, WO2014/031687, WO2013/166321, WO2013/071154, WO2013/123061 U.S. patent application publication numbers US2002/131960, US2013/287748, US2013/0149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the genetically engineered antigen receptors include a CAR as described in U.S. Pat. No. 7,446,190, and those described in International Patent Application Publication No.: WO/2014055668 A1.

Methods for administration of engineered cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

Also provided are methods that involve administering a cell therapy to a subject having a disease or condition, wherein the cell therapy contains cells that secrete an inhibitor of microglia activity. In some such examples, it is understood that such methods do not require or involve the administration of a separate inhibitor, e.g. soluble or exogenous inhibitor (e.g. separate from the cells). In some cases, the secreted inhibitor is an inhibitor of colony-stimulating factor-1 receptor (CSF1R). In some embodiments, the inhibitor is an antibody or antigen-binding fragment thereof. For example, the inhibitor is selected from emactuzumab, IMC-CS4, FPA008, LY-3022855, AMG-820, TG-3003, H27K15, 12-2D6, 2-4A5, or an antigen-binding fragment thereof.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means. The cells are administered in a dosing regimen to achieve a therapeutic effect, such as a reduction in tumor burden. Dosing and administration may depend in part on the schedule of administration of the agent, which can be administered prior to, subsequent to and/or simultaneously with initiation of administration of the therapeutic agent, e.g., T cell therapy. Various dosing schedules of the therapeutic agent, e.g. T cell therapy, include but are not limited to single or multiple administrations over various time-points, bolus administration, and pulse infusion.

a. Compositions and formulations

In some embodiments, the dose of cells of the T cell therapy, such a T cell therapy comprising cells engineered with a recombinant antigen receptor, e.g. CAR or TCR, is provided as a composition or formulation, such as a pharmaceutical composition or formulation. Such compositions can be used in accord with the provided methods, such as in the prevention or treatment of diseases, conditions, and disorders.

In some embodiments, the T cell therapy, such as engineered T cells (e.g. CAR T cells), are formulated with a pharmaceutically acceptable carrier. In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

In some cases, the cell therapy is administered as a single pharmaceutical composition comprising the cells. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

b. Dosage Schedule and Administration

In some embodiments, a dose of cells is administered to subjects in accord with the provided methods. In some embodiments, the size or timing of the doses is determined as a function of the particular disease or condition in the subject. It is within the level of a skilled artisan to empirically determine the size or timing of the doses for a particular disease in view of the provided description.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered. In some embodiments, the dose of cells is a flat dose of cells or fixed dose of cells such that the dose of cells is not tied to or based on the body surface area or weight of a subject.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸, or total such cells, or the range between any two of the foregoing values

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or about at least 1×10⁵ total recombinant receptor-expressing cells, total T cells, or total peripheral blood mononuclear cells (PBMCs), such at least or at least 1×10⁶, at least or about at least 1×10⁷, at least or about at least 1×10⁸ of such cells. In some embodiments, the number is with reference to the total number of CD3+ or CD8+, in some cases also recombinant receptor-expressing (e.g. CAR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/CAR+ or CD8+/CAR+ cells, from or from about 5×10⁵ to 1×10⁷ total CD3+/CAR+ or CD8+/CAR+ cells, or from or from about 1×10⁶ to 1×10⁷ total CD3+/CAR+ or CD8+/CAR+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷, 1×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, nor more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 1×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷ or 1×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷ or 1×10⁸ total recombinant receptor-expressing CD8+ T cells.

In the context of adoptive cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

The term “split dose” refers to a dose that is split so that it is administered over more than one day. This type of dosing is encompassed by the present methods and is considered to be a single dose. In some embodiments, the cells of a split dose are administered in a plurality of compositions, collectively comprising the cells of the dose, over a period of no more than three days.

Thus, the dose of cells may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+− and CD4+− enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some cases, the provided methods permit administration of a higher dose of such cells, which, in some cases, can increase the efficacy of the treatment. In some aspects, such higher doses of cells may cause or may likely cause a higher or greater risk of developing a toxicity or a more severe toxicity, but which risk or toxicity can be ameliorated or lessened by the provided methods when administered in a combination therapy with a microglial inhibitor as described. In some embodiments, the risk of developing a toxicity that accompanies the higher dose of cells is reduced using the method wherein an inhibitor of microglia activity is administered to the subject. In some cases, the dose of cells administered is greater than a method in which the cell therapy is administered without the inhibitor. For example, the higher dose is at least 1.5-fold, 2-fold, 3-fold, 4-fold, 5-fold or 10-fold greater.

In some embodiments, for example, the higher dose contains more than about 1×10⁶ cells, recombinant receptor (e.g. CAR)-expressing cells, T cells, and/or PBMCs per kilogram body weight of the subject, such as about or at least about 2×10⁶, 3×10⁶, 5×10⁶, 1×10⁷, 1×10⁸, or 1×10⁹ such cells per kilogram body weight of the subject. In some embodiments, the number of cells in the consecutive dose is between about 2×10⁶ cells/kg body weight of the subject and 6×10⁶ cells/kg, between about 2.5×10⁶ cells/kg and 5.0×10⁶ cells/kg, or between about 3.0×10⁶ cells/kg and about 4.0×10⁶ cells/kg, each inclusive. In some embodiments, the higher dose contains at or about 1×10⁵, at or about 2×10⁵, at or about 5×10⁵, or at or about 1×10⁶ of such cells per kilogram body weight of the subject, or a value within the range between any two of the foregoing values. In some embodiments, such values refer to numbers of recombinant receptor-expressing cells; in other embodiments, they refer to number of T cells or PBMCs or total cells administered.

In some embodiments, one or more subsequent dose of cells can be administered to the subject. In some embodiments, the subsequent dose of cells is administered greater than or greater than about 7 days, 14 days, 21 days, 28 days or 35 days after initiation of administration of the first dose of cells. The subsequent dose of cells can be more than, approximately the same as, or less than the first dose. In some embodiments, administration of the T cell therapy, such as administration of the first and/or second dose of cells, can be repeated.

In some embodiments, the dose of cells, or subsequent dose of cells, is administered subsequently to or after administration of an inhibitor of microglial cell activity, such as at a time when one or more effects of the microglia inhibitor are achieved. In some embodiments, the method involves subsequent to administering the inhibitor, but prior to administering the cell therapy, assessing a sample from the subject for alteration in the level of a factor indicative of microglia activation inhibition and/or CSF1R inhibition. For example, in some cases, the sample assessed is whole blood, serum or plasma. Exemplary methods for assessing or determining the levels or amount of factors associated with microglial cell activation inhibition and/or CSF1R inhibition are described in Section IV.

In some embodiments, initiation of administration of the cell therapy, e.g. the dose of cells or a first dose of a split dose of cells, is administered before (prior to), concurrently with or after (subsequently or subsequent to) the administration of the inhibitor of microglial cell activity.

In some embodiments, the dose of cells, or the subsequent dose of cells, is administered concurrently with or after starting or initiating administration of the inhibitor of migroglial cell activity, e.g. CSF1R inhibitor. In some embodiments, the dose of cells, or the subsequent dose of cells, is administered 0 to 90 days, such as 0 to 30 days, 0 to 15 days, 0 to 6 days, 0 to 96 hours, 0 to 24 hours, 0 to 12 hours, 0 to 6 hours, or 0 to 2 hours, 2 hours to 30 days, 2 hours to 15 days, 2 hours to 6 days, 2 hours to 96 hours, 2 hours to 24 hours, 2 hours to 12 hours, 2 hours to 6 hours, 6 hours to 90 days, 6 hours to 30 days, 6 hours to 15 days, 6 hours to 6 days, 6 hours to 96 hours, 6 hours to 24 hours, 6 hours to 12 hours, 12 hours to 90 days, 12 hours to 30 days, 12 hours to 15 days, 12 hours to 6 days, 12 hours to 96 hours, 12 hours to 24 hours, 24 hours to 90 days, 24 hours to 30 days, 24 hours to 15 days, 24 hours to 6 days, 24 hours to 96 hours, 96 hours to 90 days, 96 hours to 30 days, 96 hours to 15 days, 96 hours to 6 days, 6 days to 90 days, 6 days to 30 days, 6 days to 15 days, 15 days to 90 days, 15 days to 30 days or 30 days to 90 days after starting or initiating administration of the inhibitor of microglial cell activity, e.g. CSF1R inhibitor. In some embodiments, the dose of cells is administered at least or about at least or about 1 hour, 2 hours, 6 hours, 12 hours, 24 hours, 2 days, 3 days, 6 days, 12 days, 15 days, 30 days, 60 days or 90 days after starting or initiating administration of the inhibitor of microglial cell activity, e.g. inhibitor of CSF1R.

In some embodiments, the dose of cells is administered at least or about 4 days or more after initiating or initiating administration of the inhibitor of microglial cell activity, e.g. inhibitor of CSF1R.

In some embodiments, the administration of the inhibitor of microglial cell activity, e.g. CSF1R inhibitor, is at a time in which the prior administration of the immunotherapy (e.g. T cell therapy, such as CAR-T cell therapy) is associated with, or is likely to be associated with, a toxic outcome in the subject or a risk of a toxic outcome or potential toxic outcome in the subject. In some embodiments, the method involves, subsequent to administering the dose of cells of the T cell therapy, e.g., adoptive T cell therapy, but prior to administering the inhibitor of microglial cell activity, e.g., CSF1R inhibitor, assessing a sample from the subject for one or more toxic outcomes associated with administration of the immunotherapy, e.g., such as one or more toxic outcomes associated with CRS or neurotoxicity, such as a sign or symptom associated with a mild or moderate CRS or toxic outcome, e.g. grade 1 or grade 2. In some embodiments, such toxic outcomes include those described in Section IV. Various parameters for determining or assessing the regimen of the combination therapy are described in Section IV.

In some embodiments, administration of the inhibitor of microglial cell activity, e.g. CSF1R inhibitor, is at a time that is within about 1 day, 2 days, 3 days, four days, five days, six days or seven days after administration of the therapy and/or (ii) at or about or within 24 hours of the subject exhibiting a first sign or symptom indicative of CRS or neurotoxicity after administration of the therapy. In some embodiments, the first sign or symptom indicative or CRS or neurotoxcity is an altered biomarker, e.g. cytokine or other serum or blood biomarker, or is a fever. In some embodiments, the altered biomarker is increased or decreased in the subject by greater than or about 1.5-fold, 2-fold, 3-fold, 4-fold, 5-fold, 10-fold, 20-fold, 30-fold, 40-fold, 50-fold or more compared to in a subject, or a majority of subjects, having been administered the immunotherapy, e.g. T cell therapy, such as CAR-T cell therapy, but in the absence of the inhibitor of microglial cell activity. In some embodiments, such a sign or symptom manifests itself or is detectable by the subject, or in or from a sample from the subject, no more than 3 days, no more than 2 days, or no more than 1 day after initiation of the therapy or a first administration of the therapy.

In some embodiments, the first sign or symptom indicative of CRS or neurotoxicity is a fever, such as a fever that comprises a temperature of at least or at least about 38.0° C. In some embodiments, the fever comprises a temperature that is between or between about 38.0° C. and 42.0° C., 38.0° C. and 39.0° C., 39.0° C. and 40.0° C. or 40.0° C. and 42.0° C., each inclusive. In some embodiments, the fever is or comprises a temperature that is greater than or greater than about or is or is about 38.5° C., 39.0°, 39.5° C., 40.0° C., 41.0° C., 42.0° C. In some embodiments, the fever is a sustained fever, such as is a fever that is not reduced or not reduced by more than 1° C. after treatment with an antipyretic and/or wherein the fever has not been reduced by more than 1° C., following treatment of the subject with an antipyretic.

In some embodiments, the alteration in the level of a factor is selected from an increase in plasma CSF-1, an increase in a level of a serum enzyme, an increase in a level of serum cytokine, or a decrease in CD14dim/CD16+ nonclassical monocytes (Bendell et al. AACR-NCI-EORTC Molecular Targets and Cancer Therapeutics Abstract #A252 (2013); Rugo et al., Annals of Oncology (Supplement 4): iv 146-iv164 (2014)). In some cases, the serum enzyme is selected from alanine aminotransferase (ALT), AST, creatine kinase (CK) and LDH and the serum cytokine is selected from TNF-α, IL-6, and IL-1β (Wang et al., Annals of Translational Medicine, 2(10):136 (2015); Radi et al., Am J Pathol., 79(1):240-7 (2011); Hambleton et al., American College of Rheumatology and the Association of Rheumatology Health Professionals (ACR/ARHP) Annual Scientific Meeting, Poster #1493 (2014); Wolf et al., Division of Medicinal Chemistry Scientific Abstracts for the 250th National Meeting and Exposition, Abstract MEDI 278 (2015); international patent application publication number WO2016/069727A1).

In some embodiments, the sample is urine assessed and the alteration in the level of a factor is a decrease in the level of urinary collagen type 1 cross-linked N-telopeptide (NTX). In some embodiments, the sample is cerebrospinal fluid and biomarkers such as CD163, CCL18 (also known as pulmonary activation-regulated chemokine), CCL2 (MCP-1), Neopterin, YKL-40, CD14, CD163, and Chitotrosidase are assessed (Stilund et al., PLoS One 9(6): e98588 (2014); Lautner et al., Int J Alzheimers Dis. 2011: 939426 (2011)).

In some aspects, detecting the biomarker includes performing an in vitro assay. In some embodiments, the in vitro assay is an immunoassay, an aptamer-based assay, a histological or cytological assay, or an mRNA expression level assay. In some embodiments, the parameter or parameters for one or more of each of the one or more biomarkers are detected by an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay.

In some embodiments, the parameter for at least one of the one or more biomarkers is determined using a binding reagent that specifically binds to at least one biomarker. In some cases, the binding reagent is an antibody or antigen-binding fragment thereof, an aptamer or a nucleic acid probe.

B. Administration of Agent

The provided methods, compositions, combinations, kits and uses involve administration of an agent such as the immunomodulating or antioxidant agent and/or agent that reduces one or more microglial cell activation, activity, phenotype or function and/or agent having cytoprotective and/or antioxidant effects. In some embodiments, the methods, composition, combinations, kits and uses involve administration of an agent that is an inhibitor of microglial cell activity, e.g. a CSF1R inhibitor. In some embodiments, the agent can be administered prior to, subsequently to, during, simultaneously or near simultaneously, sequentially and/or intermittently with administration of the immunotherapy, e.g., T cell therapy, e.g., administration of T cells expressing a chimeric antigen receptor (CAR).

In some embodiments, the agent, e.g. inhibitor, in the combination therapy is an inhibitor of a microglial cell activity. In some embodiments, the administration of the inhibitor modulates the activity of microglia. In some embodiments, the inhibitor is an antagonist that inhibits the activity of a signaling pathway in microglia. In some embodiments, the microglia inhibitor affects microglial homeostasis, survival, and/or proliferation. In some embodiments, the inhibitor targets the CSF1R signaling pathway. In some embodiments, the inhibitor is an inhibitor of CSF1R. In some embodiments, the inhibitor is a small molecule. In some cases, the inhibitor is an antibody.

In some aspects, administration of the inhibitor results in one or more effects selected from an alteration in microglial homeostasis and viability, a decrease or blockade of microglial cell proliferation, a reduction or elimination of microglial cells, a reduction in microglial activation, a reduction in nitric oxide production from microglia, a reduction in nitric oxide synthase activity in microglia, or protection of motor neurons affected by microglial activation. In some embodiments, the agent alters the level of a serum or blood biomarker of CSF1R inhibition, or a decrease in the level of urinary collagen type 1 cross-linked N-telopeptide (NTX) compared to at a time just prior to initiation of the administration of the inhibitor. In some embodiments, the administration of the agent transiently inhibits the activity of microglia activity and/or wherein the inhibition of microglia activity is not permanent. In some embodiments, the administration of the agent transiently inhibits the activity of CSF1R and/or wherein the inhibition of CSF1R activity is not permanent.

In some embodiments, the agent that reduces microglial cell activity is a small molecule, peptide, protein, antibody or antigen-binding fragment thereof, an antibody mimetic, an aptamer, or a nucleic acid molecule. In some embodiments, the method involves administration of an inhibitor of microglia activity. In some embodiments, the agent is an antagonist that inhibits the activity of a signaling pathway in microglia. In some embodiments, the agent that reduces microglial cell activity affects microglial homeostasis, survival, and/or proliferation.

In some embodiments, the agent, such as the immunomodulating or antioxidant agent and/or agent that reduces one or more microglial cell activation, activity, phenotype or function and/or agent having cytoprotective and/or antioxidant effects, is or comprises an anti-inflammatory agent, an inhibitor of NADPH oxidase (NOX2); a calcium channel blocker; a sodium channel blocker; an agent that inhibits GM-CSF; an agent that inhibits CSF1R, specifically binds CSF-1; an agent that specifically binds IL-34; an agent that inhibits the activation of nuclear factor kappa B (NF-κB); an agent that activates a CB₂ receptor and/or is a CB₂ agonist; a phosphodiesterase inhibitor; an agent that inhibits microRNA-155 (miR-155); an agent that upregulates microRNA-124 (miR-124); an agent that inhibits nitric oxide production in microglia; an agent that inhibits nitric oxide synthase; an agent that activates or promotes the activation, translocation or upregulation of the transcription factor NRF2 (also called nuclear factor (erythroid-derived 2)-like 2, or NFE2L2) and/or of an NRF2-regulated or NRF2-related pathway; an agent that activates, promoters or upregulates expression of one or more genes having or capable of being activated by an antioxidant response element (ARE); an agent that activates or promotes phase II detoxicfication, anti-oxidant enzymes or anti-inflammatory or antioxidant activities thereof; an agent that promotes anti-oxidant or anti-inflammatory pathways; or an agent that binds to or results in the modification of kelch-like ECH-associated protein 1 (KEAP1) and/or hydroxycarboxylic acid receptor 2 (HCAR2).

In some embodiments, the agent is or comprises one or more fumarate esters, such as an agent that is or comprises a dimethyl fumarate (DMF), and/or is or comprises an agent that promotes the accumulation or presence of or is capable of being metabolized into monomethyl fumarate (MMF). Without being bound by theory, in some embodiments, DMF has cryoprotective and/or anti-oxidant effects and can modify KEAP1, which may result in translocation of NRF2 into the nucleus of a cell, which in turn may result in NRF2 binding to the antioxidant response element (ARE) in a promoter region of one or more genes such as phase II genes, stimulating the transcription thereof, which may result in induction of phase II detoxification and/or anti-oxidant enzymes and/or the anti-inflammatory and antioxidant activities thereof. In some embodiments, DMF is metabolized into MMF, which in some aspects may cross the blood brain barrier and/or have an impact on cells or factors within the CNS. In some aspects, DMF or its metabolite may bind to HCAR2, which may lead to the reduction in inflammatory function of, and/or induction of an anti-inflammatory phenotype of, one or more cells such as microglial cells or astrocytes. See Prosperini and Pontecorvo; Therapeutics and Clinical Risk Management 2016:12 339-350.

In some embodiments, the agent, such as the agent that reduces microglial cell activity, targets CSF1 (also called macrophage colony-stimulating factor MCSF). In some embodiments, the agent that reduces microglial cell activity affects MCSF-stimulated phosphorylation of the M-CSF receptor (Pryer et al. Proc Am Assoc Cancer Res, AACR Abstract nr DDT02-2 (2009)). In some cases, the agent that reduces microglial cell activity is MCS110 (international patent application publication number WO2014001802; Clinical Trial Study Record Nos.:A1 NCT00757757; NCT02807844; NCT02435680; NCT01643850).

In some embodiments, the agent, such as the agent that reduces or modulates a microglial cell activity, is a small molecule that targets the CSF1 pathway. In some embodiments, the agent is a small molecule that binds CSF1R. In some embodiments, the agent is a small molecule which inhibits CSF1R kinase activity by competing with ATP binding to CSF1R kinase. In some embodiments, the agent is a small molecule which inhibits the activation of the CFS1R receptor. In some cases, the binding of the CSF-1 ligand to the CSF1R is inhibited. In some embodiments, the agent is any of the inhibitors described in US Patent Application Publication Number US20160032248.

In some embodiments, the agent is a small molecule inhibitor selected from PLX-3397, PLX7486, JNJ-40346527, JNJ28312141, ARRY-382, PLX73086 (AC-708), DCC-3014, AZD6495, GW2580, Ki20227, BLZ945, PLX647, PLX5622. In some embodiments, the agent is any of the inhibitors described in Conway et al., Proc Natl Acad Sci USA, 102(44):16078-83 (2005); Dagher et al., Journal of Neuroinflammation, 12:139 (2015); Ohno et al., Mol Cancer Ther. 5(11):2634-43 (2006); von Tresckow et al., Clin Cancer Res., 21(8) (2015); Manthey et al. Mol Cancer Ther. (8(11):3151-61 (2009); Pyonteck et al., Nat Med. 19(10): 1264-1272 (2013); Haegel et al., Cancer Res AACR Abstract nr 288 (2015); Smith et al., Cancer Res AACR Abstract nr 4889 (2016); Clinical Trial Study Record Nos.: NCT01525602; NCT02734433; NCT02777710; NCT01804530; NCT01597739; NCT01572519; NCT01054014; NCT01316822; NCT02880371; NCT02673736; international patent application publication numbers WO2008063888A2, WO2006009755A2, US patent application publication numbers US20110044998, US 2014/0065141, and US 2015/0119267.

In some embodiments, the agent such as the agent that reduces a microglial cell activity is 4-((2-(((1R,2R)-2-hydroxycyclohexyl)amino)benzo[d]thiazol-6-yl)oxy)-N-methylpicolinamide (BLZ945) or a pharmaceutically acceptable salt thereof or derivatives thereof. In some embodiments, the agent is the following compound:

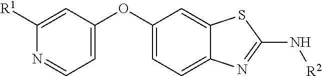

wherein R1 is an alkyl pyrazole or an alkyl carboxamide, and R2 is a hydroxycycloalkyl or a pharmaceutically acceptable salt thereof.

In some embodiments, the agent that reduces microglial cell activity is 5-((5-chloro-1H-pyrrolo[2,3-b]pyridin-3-yl)methyl)-N-((6-(trifluoromethyl)pyridin-3-yl)methyl)pyridin-2-amine, N-[5-[(5-Chloro-1H-pyrrolo[2,3-b]pyridin-3-yl)methyl]-2-pyridinyl]-6-(trifluoromethyl)-3-pyridinemethanamine) (PLX 3397) or a pharmaceutically acceptable salt thereof or derivatives thereof. In some embodiments, the agent is 5-(1H-Pyrrolo[2,3-b]pyridin-3-ylmethyl)-N-[[4-(trifluoromethyl)phenyl]methyl]-2-pyridinamine dihydrochloride (PLX647) or a pharmaceutically acceptable salt thereof or derivatives thereof. In some embodiments, the agent that reduces microglial cell activity is the following compound:

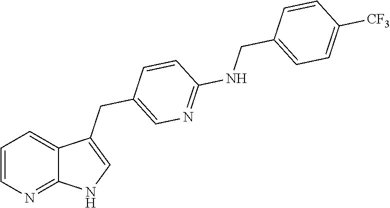 [SMILES: FC(F)(F)c1ccc(CNc2ccc(Cc3c[nH]c4ncccc34)cn2)cc1]

or a pharmaceutically acceptable salt thereof. In some embodiments, the agent that reduces microglial cell activity is the following compound:

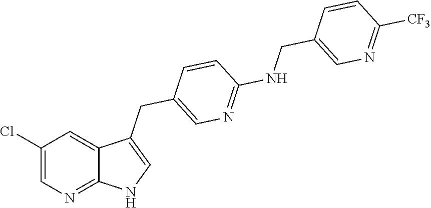 [SMILES: FC(F)(F)c1ccc(CNc2ccc(Cc3c[nH]c4ncc(Cl)cc34)cn2)cn1]

or a pharmaceutically acceptable salt thereof. In some embodiments, the agent is any of the inhibitors described in U.S. Pat. No. 7,893,075.

In some embodiments, the agent that reduces microglial cell activity is 4-cyano-N-[2-(1-cyclohexen-1-yl)-4-[1-[(dimethylamino)acetyl]-4-piperidinyl]phenyl]-1H-imidazole-2-carboxamide monohydrochloride (JNJ28312141) or a pharmaceutically acceptable salt thereof or derivatives thereof. In some embodiments, the agent is the following compound:

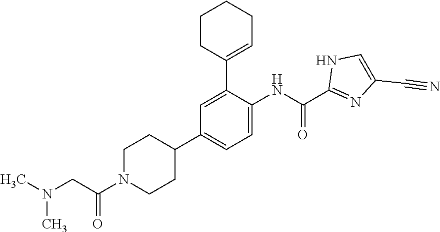 [SMILES: CN(C)CC(=O)N1CCC(c2ccc(NC(=O)c3nc(C#N)c[nH]3)c(C3=CCCCC3)c2)CC1]

or a pharmaceutically acceptable salt thereof. In some embodiments, the agent is any of the inhibitors described in U.S. Pat. No. 7,645,755.

In some embodiments, the agent that reduces microglial cell activity is 1H-Imidazole-2-carboxamide, 5-cyano-N-(2-(4,4-dimethyl-1-cyclohexen-1-yl)-6-(tetrahydro-2,2,6,6-tetramethyl-2H-pyran-4-yl)-3-pyridinyl)-, 4-Cyano-1H-imidazole-2-carboxylic acid N-(2-(4,4-dimethylcyclohex-1-enyl)-6-(2,2,6,6-tetramethyltetrahydropyran-4-yl)pyridin-3-yl)amide, 4-Cyano-N-(2-(4,4-dimethylcyclohex-1-en-1-yl)-6-(2,2,6,6-tetramethyl-tetrahydro-2H-pyran-4-yl)pyridin-3-yl)-1H-imidazole-2-carboxamide (JNJ-40346527) or a pharmaceutically acceptable salt thereof or derivatives thereof. In some embodiments, the agent is the following compound:

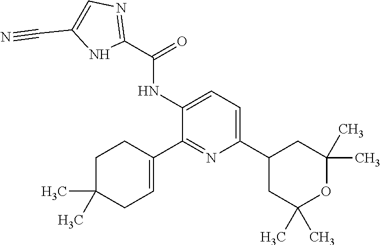 [SMILES: CC1(C)CC=C(c2nc(C3CC(C)(C)OC(C)(C)C3)ccc2NC(=O)c2ncc(C#N)[nH]2)CC1]

or a pharmaceutically acceptable salt thereof.

In another embodiment, the agent that reduces microglial cell activity is 5-(3-Methoxy-4-((4-methoxybenzyl)oxy)benzyl)pyrimidine-2,4-diamine (GW2580) or a pharmaceutically acceptable salt thereof or derivatives thereof. In some embodiments, the agent is the following compound:

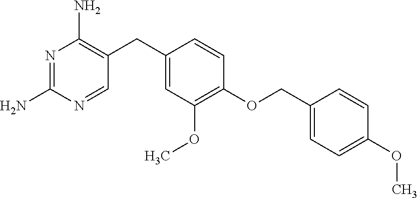 [SMILES: COc1ccc(COc2ccc(Cc3cnc(N)nc3N)cc2OC)cc1]

or a pharmaceutically acceptable salt thereof (international patent application publication number WO2009099553).

In some embodiments, the agent that reduces microglial cell activity is 4-(2,4-difluoroanilino)-7-ethoxy-6-(4-methylpiperazin-1-yl)quinoline-3-carboxamide (AZD6495) or a pharmaceutically acceptable salt thereof or derivatives thereof. In some embodiments, the agent is the following compound:

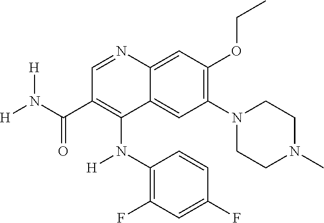 [SMILES: CCOc1cc2ncc(C(N)=O)c(Nc3ccc(F)cc3F)c2cc1N1CCN(C)CC1]

or a pharmaceutically acceptable salt thereof.

In some embodiments, the agent that reduces microglial cell activity is N-{4-[(6,7-dimethoxy-4-quinolyl)oxy]-2-methoxyphenyl}-N0-[1-(1,3-thiazole-2-yl)ethyl]urea (Ki20227) or a pharmaceutically acceptable salt thereof or derivatives thereof. In some embodiments, the agent is the following compound:

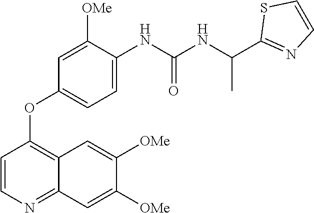 [SMILES: COc1cc(Oc2ccnc3cc(OC)c(OC)cc23)ccc1NC(=O)NC(C)c1nccs1]

or a pharmaceutically acceptable salt thereof.

In some embodiments, the agent that reduces or modulates a microglial cell activity is an antibody that targets the CSF1 pathway. In some embodiments, the agent is an antibody that binds CSF1R. In some embodiments, the anti-CSF1R antibody blocks CSF1R dimerization. In some embodiments, the anti-CSF1R antibody blocks the CSF1R dimerization interface that is formed by domains D4 and D5 (Ries et al. Cancer Cell 25(6):846-59 (2014)). In some cases, the agent is selected from emactuzumab (RG7155; R05509554), Cabiralizumab (FPA-008), LY-3022855 (IMC-CS4), AMG-820, TG-3003, MCS110, H27K15, 12-2D6, 2-4A5 (Rovida and Sbarba, J Clin Cell Immunol. 6:6 (2015); Clinical Trial Study Record Nos.: NCT02760797; NCT01494688; NCT02323191; NCT01962337; NCT02471716; NCT02526017; NCT01346358; NCT02265536; NCT01444404; NCT02713529, NCT00757757; NCT02807844; NCT02435680; NCT01643850).

In some embodiments, the agent that reduces or modulates a microglial cell activity or activation is a tetracycline antibiotic. For example, the agent affects IL-1b, IL-6, TNF-α, or iNOS concentration in microglia cells (Yrjänheikki et al. PNAS 95(26): 15769-15774 (1998); Clinical Trial Study Record No: NCT01120899). In some embodiments, the agent is an opioid antagonist (Younger et al. Pain Med. 10(4):663-672 (2009.) In some embodiments, the agent reduces glutamatergic neurotransmission (U.S. Pat. No. 5,527,814). In some embodiments, the agent modulates NFkB signaling (Valera et al J. Neuroinflammation 12:93 (2015); Clinical Trial Study Record No: NCT00231140). In some embodiments, the agent targets cannabinoid receptors (Ramirez et al. J. Neurosci 25(8):1904-13(2005)). In some embodiments, the agent is selected from minocycline, naloxone, riluzole, lenalidomide, and a cannabinoid (optionally WIN55 or 212-2).

Nitric oxide production from microglia may, in some cases, result in or increase neurotoxicity. In some embodiments, the agent modulates or inhibits nitric oxide production from microglia. In some embodiments, the agent inhibits nitric oxide synthase (NOS). In some embodiments, the NOS inhibitor is Ronopterin (VAS-203), also known as 4-amino-tetrahydrobiopterin (4-ABH4). In some embodiments, the NOS inhibitor is cindunistat, A-84643, ONO-1714, L-NOARG, NCX-456, VAS-2381, GW-273629, NXN-462, CKD-712, KD-7040, or guanidinoethyldisulfide. In some embodiments, the agent is any of the inhibitors described in Höing et al., Cell Stem Cell. 2012 Nov. 2; 11(5):620-32.

In some embodiments, the agent blocks T cell trafficking, such as to the central nervous system. In some embodiments, blocking T cell trafficking can reduce or prevent immune cells from crossing blood vessel walls into the central nervous system, including crossing the blood-brain barrier. In some cases, activated antigen-specific T cells produce proinflammatory cytokines, including IFN-γ and TNF, upon reactivation in the CNS, leading to activation of resident cells such as microglia and astrocytes. See Kivisäkk et al., Neurology. 2009 Jun. 2; 72(22): 1922-1930. Thus, in some embodiments, sequestering activated T cells from microglial cells, such as by blocking trafficking and/or inhibiting the ability of such cells to cross the blood-brain barrier, can reduce or eliminate microglial activation. In some embodiments, the agent inhibits adhesion molecules on immune cells, including T cells. In some embodiments, the agent inhibits an integrin. In some embodiments, the integrin is alpha-4 integrin. In some embodiments, the agent is natalizumab (Tysabri®). In some embodiments, the agent modulates a cell surface receptor. In some embodiments, the agent modulates the sphingosine-1-phosphate (S1P) receptor, such as S1PR1 or S1PR5. In some embodiments, the agent causes the internalization of a cellular receptor, such as a sphingosine-1-phosphate (S1P) receptor, such as S1PR1 or S1PR5. In some embodiments, the agent is fingolimod (Gilenya®) or ozanimod (RPC-1063).

Without wishing to be bound by theory the transcription factor NRF2 can in some aspects regulate the anti-oxidant response, for example, by turning on genes that contain a cis-acting element in their promoter region. An example of such an element includes an antioxidant response element (ARE). In some embodiments, the agent activates NRF2 or an NRF2-related pathway or promotes modulation of NRF2 such as translocation thereof into the nucleus. In some embodiments, activating NRF2 in microglial cells reduces the microglial cells' responsiveness to IFN and LPS. In some embodiments, activating NRF2 inhibits, slows, or reduces demyelination, axonal loss, neuronal death, and/or oligodendrocyte death. In some embodiments, the agent upregulates the cellular cytoprotective pathway regulated by NRF2. In some embodiments, the agent that activates NRF2 is a fumaric acid ester, such as a dimethyl fumarate (DMF), such as the compound referred to by the name Tecfidera®.

In some embodiments, the agent is the following compound:

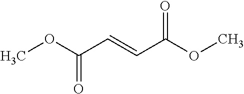

or a pharmaceutically acceptable salt thereof.

In some embodiments, the agent is any of the inhibitors described in U.S. Pat. No. 8,399,514. In some embodiments, the agent is any of the inhibitors described in Höing et al., Cell Stem Cell. 2012 Nov. 2; 11(5):620-32.

In some embodiments, the agent is (4S,4aS,5aR,12aS)-4,7-bis(dimethylamino)-3,10,12,12a-tetrahydroxy-1,11-dioxo-1,4,4a,5,5a,6,11,12a-octahydrotetracene-2-carboxamide (Minocycline) or a pharmaceutically acceptable salt thereof or derivatives thereof. In some embodiments, the agent is any of the compounds described in US patent application publication number US20100190755. In some embodiments, the agent is the following compound:

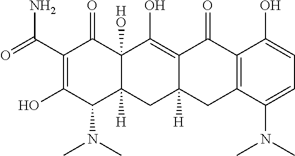 [SMILES: CN(C)c1ccc(O)c2c1C[C@H]1C[C@H]3[C@H](N(C)C)C(O)=C(C(N)=O)C(=O)[C@@]3(O)C(O)=C1C2=O]

or a pharmaceutically acceptable salt thereof.

In some embodiments, the agent is 3-(7-amino-3-oxo-1H-isoindol-2-yl)piperidine-2,6-dione (lenalidomide) or a pharmaceutically acceptable salt thereof or derivatives thereof. In some embodiments, the agent is the following compound:

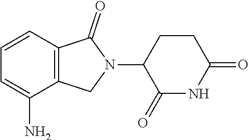 [SMILES: Nc1cccc2c1CN(C1CCC(=O)NC1=O)C2=O]

or a pharmaceutically acceptable salt thereof.

In some embodiments, the agent is 4R,4aS,7aR,12bS)-4a,9-dihydroxy-3-prop-2-enyl-2,4,5,6,7a,13-hexahydro-1H-4,12-methanobenzofuro[3,2-e]isoquinoline-7-one (naloxone) or a pharmaceutically acceptable salt thereof or derivatives thereof. In some embodiments, the agent is any of the compounds described in U.S. Pat. No. 8,247,425. In some embodiments, the agent is the following compound:

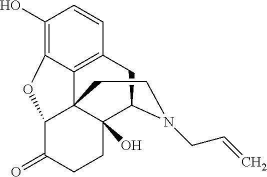 [SMILES: C=CCN1CC[C@]23c4c5ccc(O)c4O[C@H]2C(=O)CC[C@@]3(O)[C@H]1C5]

or a pharmaceutically acceptable salt thereof.

In some embodiments, the agent is 2-amino-6-(trifluoromethoxy)benzothiazole, 6-(trifluoromethoxy)benzo[d]thiazol-2-amine, or 6-(trifluoromethoxy)-1,3-benzothiazol-2-amine (riluzole) or a pharmaceutically acceptable salt thereof or derivatives thereof as described in U.S. Pat. No. 5,527,814. In some embodiments, the agent is the following compound:

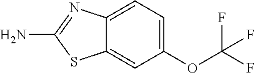 [SMILES: Nc1nc2ccc(OC(F)(F)F)cc2s1]

or a pharmaceutically acceptable salt thereof.

In some embodiments, the agent is a modulator of a signaling pathway in microglia. In some cases, the agent reduces microglia singling. In some embodiments, the agent is a GM-CSF (CSF2) inhibitor. In other embodiments, the agent is an ion channel blocker. In some specific embodiments, the agent is a calcium channel blocker. For example, in some specific examples, the agent is a dihydropyridine calcium channel blocker. In some embodiments, the agent is a microRNA inhibitor. For example, the agent targets miR-155. In some embodiments, the agent that reduces microglial cell activation is selected from MOR103, Nimodipine, IVIg, and LNA-anti-miR-155 (Butoxsky et al. Ann Neurol., 77(1):75-99 (2015) and Sanz et al., Br J Pharmacol. 167(8): 1702-1711 (2012); Winter et al., Ann Clin and Transl Neurol. 2328-9503 (2016); Clinical Trial Study Record Nos.: NCT01517282, NCT00750867).

In some embodiments, the agent is 3-(2-methoxyethyl) 5-propan-2-yl 2,6-dimethyl-4-(3-nitrophenyl)-1,4-dihydropyridine-3,5-dicarboxylate (nimodipine) or a pharmaceutically acceptable salt thereof or derivatives thereof. In some embodiments, the agent is any of the inhibitors described in U.S. Pat. No. 3,799,934. In some embodiments, the agent is the following compound:

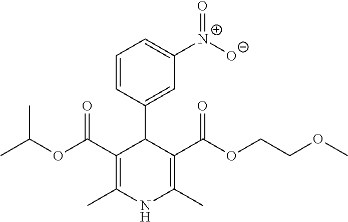 [SMILES: COCCOC(=O)C1=C(C)NC(C)=C(C(=O)OC(C)C)C1c1cccc([N+](=O)[O-])c1]

or a pharmaceutically acceptable salt thereof.

In some cases, the agent is administered in a form that only affects to central nervous system and/or does not affect tumor-associated macrophages. In some embodiments, the agent promotes microglia quiescence but does not eliminate or reduce the number of microglia. In some embodiments, the method involves inhibiting microglia activity specifically in the brain such as described in Ponomarev et al., Nature Medicine, (1):64-70 (2011)

Exemplary agents, such as agents that reduce microglial cell activation and/or modulate or reduce or alter one or more activities or functions thereof and/or anti-inflammatory agents or antioxidant gents, and exemplary dosing regimens for administering such agents, are set forth in Table 1 below.

TABLE 1
Exemplary agents and exemplary dosage regimens thereof

| Exemplary | Type of | Molecular | |
|---|---|---|---|
| Inhibitor | Molecule | Target(s) | Exemplary Dosing Regimen(s) |
| Pexidartinib | small molecule | CSF1R; c-Kit; | 200 mg tablets, twice daily for 28 days; |
| (PLX3397) | | FLT3 | Administer daily as split dose regimen, |
| | | | five dose-levels possible in dose |
| | | | escalation part: 400 mg 5 days on 2 days |
| | | | off (intermittent schedule), 400 mg, 600 |
| | | | mg, 800 mg or 1000 mg; 1000 mg/day |
| | | | for 2 weeks then 800 mg/day for 22 |
| | | | weeks |
| Emactuzumab | monoclonal | CSF1R | 100-3000 mg once every 2 weeks |
| (RG1755; | antibody | | |
| RO5509554) | | | |
| Cabiralizumab | antibody | CSF1R | Intravenous infusion over 30 minutes |
| (FPA-008) | | | every 2 weeks |
| LY-3022855 | monoclonal | CSF1R | 1.25 mg/kg intravenous delivery every 2 |
| (IMC-CS4) | antibody | | weeks for 6 weeks |
| JNJ-40346527 | small molecule | CSF1R | 100 mg twice daily for 12 weeks; 100- |
| | | | 1000 mg capsule daily |
| MCS110 | antibody | MCSF (CSF1) | Up to 4 doses of 10 mg/kg MCS110 |
| | | | administered intravenously once every 4 |
| | | | weeks starting at Day 1 |
| MOR103 | antibody | GM-CSF | 6 doses of 0.5-2.0 mg/kg over 70 days |
| IVIg | immunoglobulin | Unknown | Intravenous infusion of 0.4 g/kg each |
| | | | month for 6 months |
| Minocyline | small molecule | broad spectrum | Oral dose of 100 mg of minocycline |
| | | antibiotic: IL-1b; | twice daily for 24 months |
| | | IL-6, TNF-a; | |
| | | iNOS | |
| Naloxone | small molecule | Opioid receptors | 4.5 mg naltrexone hydrochloride |
| | | | capsules once/day for 8 weeks |
| Lenalidomide/thalidomide | small molecule | NFkB signaling | 100-400 mg daily |
| Riluzole | small molecule | Glutamate release | 50 mg twice daily |
| | | by microglia | |
| Cannabinoids/cannabidiol | small molecule | cannabinoid | Orally 10 mg/kg/day for 6 weeks |
| (e.g. WIN55, 212-2) | | receptors | (average of 700 mg/day) |
| Dimethyl | small molecule | Nrf2 signaling | Starting dose of 120 mg taken orally |
| fumarate | | | twice/day for 7 days. Dose increased to |
| (Tecfidera ®). | | | 240 mg taken orally twice/day thereafter |
| natalizumab | antibody | alpha-4 integrin | 300 mg infused intravenously over |
| (Tysabri ®) | | | one hour, every four weeks |
| fingolimod | small molecule | S1P receptors, | 0.5 mg orally once-daily |
| (Gilenya ®) | | including | |
| | | S1PR1 | |
| ozanimod | small molecule | S1PR1 and | 0.25 mg, 0.5 mg, or 1 mg once daily |
| (RPC-1063) | | S1PR5 | |

1. Compositions and Formulations

In some aspects, the choice of carrier is determined in part by the particular agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980).

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). The compositions containing the inhibitor can also be lyophilized.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, the inhibitors are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

2 Dosage Schedule of Agent

In some embodiments, the additional agent such as the anti-inflammatory agent and/or antioxidant agent and/or agent that reduces microglial cell activity is administered sequentially, intermittently, or at the same time as or in the same composition as cells for adoptive cell therapy. For example, the agent can be administered prior to, during, simultaneously with, or after administration of the cell therapy. In some embodiments, the method involves administering the agent prior to administration of the cell therapy. In some embodiments, the agent is not further administered after initiation of the cell therapy. In other embodiments, the method further involves administering the agent after administration of the cell therapy. In some cases, the dosage schedule comprises administering the agent prior to and after initiation of the cell therapy. In some embodiments, the initiation of administration of the agent is at a time point that is greater than or greater than about 1 hour, 2 hours, 6 hours, 12 hours, 24 hours, 3 days, 6 days, 12 days, 15 days, 30 days, 60 days or 90 days prior to initiation of the administration of the cell therapy. In some aspects, the initiation of administration of the agent is more than 4 days before the administration of the cell therapy.

In some embodiments, the agent is administered prior to the administration of the cells in sufficient time to reduce the number of microglia or one or more function or activity thereof such as to reduce production of inflammatory or stress response factors or inflammatory effects thereof, in the subject. In some cases, for example, the agent is given at least 7 days to at least 21 days prior to the administration of the cells, e.g., to allow the agent to sufficiently deplete or reduce the number of microglia in the subject or otherwise impact inflammatory function thereof (Dagher et al., Journal of Neuroinflammation, 12:139 (2015)). In some embodiments, the initiation of administration of the agent is 30 days before the administration of the cell therapy.

In some embodiments, the agent such as the agent that reduces microglial cell activity is administered daily, every other day, once a week or only one time prior to initiation of administration of the cell therapy. In some aspects, the agent is administered until the risk of a toxic outcome or symptom in the subject from administration of the cell therapy has subsided or is not present. In some embodiments, the agent is administered for a time period up to 2 days, up to 7 days, up to 14 days, up to 21 days, up to 28 days, up to 35 days or up to 42 days after initiation of the administration of the cell therapy.

In some embodiments, agent such as the agent that reduces microglial cell activity is administered daily, every other day, once a week or only one time after initiation of administration of the cell therapy for the time period. In some embodiments, the administration of the cell therapy is at a time after the number of microglial cells or an inflammatory or stress effect thereof is reduced or eliminated in the subject compared to just prior to initiation of administration of the inhibitor. In some embodiments, the administration of the cell therapy is at a time in which there exists an alteration in the level of a factor indicative of CSF1R inhibition in a sample from the subject compared to just prior to initiation of administration of the inhibitor.

In some embodiments, the agent is independently administered in a dosage amount of from or from about 0.2 mg per kg body weight of the subject (mg/kg) to 200 mg/kg, 0.2 mg/kg to 100 mg/kg, 0.2 mg/kg to 50 mg/kg, 0.2 mg/kg to 10 mg/kg, 0.2 mg/kg to 1.0 mg/kg, 1.0 mg/kg to 200 mg/kg, 1.0 mg/kg to 100 mg/kg, 1.0 mg/kg to 50 mg/kg, 1.0 mg/kg to 10 mg/kg, 10 mg/kg to 200 mg/kg, 10 mg/kg to 100 mg/kg, 10 mg/kg to 50 mg/kg, 50 mg/kg to 200 mg/kg, 50 mg/kg to 100 mg/kg or 100 mg/kg to 200 mg/kg; or the agent is administered, or each administration of the agent is independently administered, in a dosage amount of from or from about 25 mg to 2000 mg, 25 mg to 1000 mg, 25 mg to 500 mg, 25 mg to 200 mg, 25 mg to 100 mg, 25 mg to 50 mg, 50 mg to 2000 mg, 50 mg to 1000 mg, 50 mg to 500 mg, 50 mg to 200 mg, 50 mg to 100 mg, 100 mg to 2000 mg, 100 mg to 1000 mg, 100 mg to 500 mg, 100 mg to 200 mg, 200 mg to 2000 mg, 200 mg to 1000 mg, 200 mg to 500 mg, 500 mg to 2000 mg, 500 mg to 1000 mg or 1000 mg to 2000 mg, each inclusive. In some aspects, the agent is administered, or each administration of the agent is independently administered, in a dosage amount of at least or at least about or about 0.2 mg per kg body weight of the subject (mg/kg), 1 mg/kg, 3 mg/kg, 6 mg/kg, 10 mg/kg, 20 mg/kg, 30 mg/kg, 50 mg/kg, 100 mg/kg or 200 mg/kg; or the agent is administered, or each administration of the agent is independently administered, in a dosage amount of at least or at least about 25 mg, 50 mg, 100 mg, 200 mg, 400 mg, 500 mg, 600 mg, 800 mg, 1000 mg, 1200 mg, 1600 mg or 2000 mg. The agent is administered daily in a dosage amount of at least or at least about 25 mg/day, 50 mg/day, 100 mg/day, 200 mg/day, 400 mg/day, 500 mg/day, 600 mg/day, 800 mg/day, 1000 mg/day, 1200 mg/day, 1600 mg/day or 2000/day mg. In some embodiments, exemplary dosages are set forth in Table 1.

In some embodiments, the method involves administering to the subject a therapeutically effective amount of an inhibitor of colony-stimulating factor-1 receptor (CSF1R) in a dosage schedule in which a clinical risk for neurotoxicity or cytokine release syndrome (CRS) is not present or is reduced compared to an alternative dosing regimen in which the subject is administered the cell therapy without having been administered the inhibitor. In some embodiments, the method involves administering to the subject a therapeutically effective amount of an inhibitor of colony-stimulating factor-1 receptor (CSF1R) in a dosage schedule in which cerebral edema is not present or is reduced compared to an alternative dosing regimen in which the subject is administered the cell therapy without having been administered the inhibitor.

In some embodiments, the method involves administering to the subject a therapeutically effective amount of an inhibitor of colony-stimulating factor-1 receptor (CSF1R) in a dosage schedule in which a biochemical readout evidencing neurotoxicity or CRS is not present or is reduced compared to an alternative dosing regimen in which the subject is administered the cell therapy without having been administered the inhibitor. In some aspects, the biochemical readout is a serum level of a factor indicative of neurotoxicity or CRS. In some cases, the biochemical readout is reduced by greater than or greater than about 1.5-fold, 2-fold, 3-fold, 4-fold, 5-fold, 10-fold, 20-fold or 50-fold.

In some embodiments, the subject is administered a therapeutically effective amount of the additional agent such as the anti-inflammatory or anti-oxidative stress agent or inhibitor of microglia activity, after the subject exhibits a clinical sign or symptom of a toxicity-related outcome. For example, in some cases, the outcome is selected from the group consisting of fever, hypotension, hypoxia, neurologic disturbances, or a serum level of an inflammatory cytokine or C reactive protein (CRP). In some aspects, the outcome is associated with neurotoxicity such as confusion, delirium, expressive aphasia, obtundation, myoclonus, lethargy, altered mental status, convulsions, seizure-like activity, seizures (optionally as confirmed by electroencephalogram), elevated levels of beta amyloid, elevated levels of glutamate, and elevated levels of oxygen radicals. Among the factors, e.g., serum factors, indicative of CRS are inflammatory cytokines such as IFNγ, GM-CSF, TNFα, IL-6, IL-10, IL-1β, IL-8, IL-2, MIP-1, Flt-3L, fracktalkine, and IL-5. In some embodiments, the inhibitor is administered at a time at which a serum level of a factor indicative of neurotoxicity in the subject indicates the development of neurotoxicity as compared to the serum level of the indicator in the subject immediately prior to said administration of the cells.

In some embodiments, the inhibitor can be administered greater than 4 hours, 5 hours, 6 hours, 7 hours, 8 hours, 9 hours, 10 hours, 11 hours, 12 hours, 18 hours, 24 hours, 36 hours, 2 days, 3 days, 4 days, or 5 days or more following administration of the cell therapy. In some of such embodiments, the inhibitor may be administered no later than 5 hours, 6 hours, 7 hours, 8 hours, 9 hours, 10 hours, 11 hours, 12 hours, 18 hours, 24 hours, 36 hours, 2 days, 3 days, 4 days, or 5 days or more following administration of the cell therapy.

In some embodiments, the method involves assessing a biological sample from the subject for biomarkers or factors indicative of effects of the agent such as the microglia inhibitor. In some aspects, the assessment of the biological sample may help determine or modify the dosage schedule of the inhibitor. In some embodiments, the method involves subsequent to administering the inhibitor but prior to administering the cell therapy, assessing a sample from the subject for alteration in the level of a factor indicative of inflammation, oxidative stress or microglia activation inhibition and/or CSF1R inhibition. For example, in some cases, the sample assessed is whole blood, serum or plasma.

In some embodiments, the alteration in the level of a factor is selected from an increase in plasma CSF-1, an increase in a level of a serum enzyme, an increase in a level of serum cytokine, or a decrease in CD14dim/CD16+ nonclassical monocytes (Bendell et al. AACR-NCI-EORTC Molecular Targets and Cancer Therapeutics Abstract #A252 (2013); Rugo et al., Annals of Oncology (Supplement 4): iv 146-iv164 (2014)). In some cases, the serum enzyme is selected from alanine aminotransferase (ALT), AST, creatine kinase (CK) and LDH and the serum cytokine is selected from TNF-α, IL-6, and IL-1β (Wang et al., Annals of Translational Medicine, 2(10):136 (2015); Radi et al., Am J Pathol., 79(1):240-7 (2011); Hambleton et al., American College of Rheumatology and the Association of Rheumatology Health Professionals (ACR/ARHP) Annual Scientific Meeting, Poster #1493 (2014); Wolf et al., Division of Medicinal Chemistry Scientific Abstracts for the 250th National Meeting and Exposition, Abstract MEDI 278 (2015); international patent application publication number WO2016/069727A1).

In some embodiments, the sample is urine assessed and the alteration in the level of a factor is a decrease in the level of urinary collagen type 1 cross-linked N-telopeptide (NTX). In some embodiments, the sample is cerebrospinal fluid and biomarkers such as CD163, CCL18 (also known as pulmonary activation-regulated chemokine), CCL2 (MCP-1), Neopterin, YKL-40, CD14, CD163, and Chitotrosidase are assessed (Stilund et al., PLoS One 9(6): e98588 (2014); Lautner et al., Int J Alzheimers Dis. 2011: 939426 (2011)).

In some aspects, detecting the biomarker includes performing an in vitro assay. In some embodiments, the in vitro assay is an immunoassay, an aptamer-based assay, a histological or cytological assay, or an mRNA expression level assay. In some embodiments, the parameter or parameters for one or more of each of the one or more biomarkers are detected by an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay.

In some embodiments, the parameter for at least one of the one or more biomarkers is determined using a binding reagent that specifically binds to at least one biomarker. In some cases, the binding reagent is an antibody or antigen-binding fragment thereof, an aptamer or a nucleic acid probe.

In some embodiments, the administration of the inhibitor is continued until the risk of developing toxicity in the subject is diminished. In some cases, the dose of cells is not administered until the risk of developing toxicity is determined by the assessment of the biomarkers to be diminished. In some embodiments wherein the administration of the inhibitor is continued after the dose of cells has been administered, the method involves further assessing a biological sample from the subject for biomarkers or factors to assess the effects such as the effects of the microglia inhibitor after the administration of the dose of cells.

In some embodiments, assessment of the effects such as effects of the microglia inhibitor is accomplished using neuroimaging. In some embodiments, positron emission tomography (PET) and magnetic resonance (MR) imaging are used to detect microglia in the brain. In some aspects, imaging is used to detect neuroinflammation. Imaging of microglia may be accomplished by using ligands that bind to translocator protein-18 kDa (TSPO). Exemplary ligands that bind to TSPO for use in neuroimaging of microglia include ¹¹C PBR28, ¹¹C isoquinoline (R)-PK11195, ¹¹C vinpocetine, ¹¹C DAA1106 as discussed in Lautner et al. Int J Alzheimers Dis. 2011: 939426 (2011). In some embodiments, agents for imaging brain microglia activity in vivo include the use of iron oxide nanoparticles and ultra-small super paramagnetic particles that are phagocytosed (Venneti et al., Glia 61(1):10-23 (2013)).

C. Lymphodepleting Treatment

In some aspects, the provided methods can further include administering one or more lymphodepleting therapies, such as prior to or simultaneous with initiation of administration of the immunotherapy, such as a T cell therapy (e.g. CAR-expressing T cells) or a T cell-engaging therapy. In some embodiments, the lymphodepleting therapy comprises administration of a phosphamide, such as cyclophosphamide. In some embodiments, the lymphodepleting therapy can include administration of fludarabine. In some embodiments, fludarabine is excluded in the lymphodepleting therapy. In some embodiments, a lymphodepleting therapy is not administered.

Preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See, e.g., Dudley et al., Science, 298, 850-54 (2002); Rosenberg et al., Clin Cancer Res, 17(13):4550-4557 (2011). Likewise, in the context of CAR+ T cells, several studies have incorporated lymphodepleting agents, most commonly cyclophosphamide, fludarabine, bendamustine, or combinations thereof, sometimes accompanied by low-dose irradiation. See Han et al. Journal of Hematology & Oncology, 6:47 (2013); Kochenderfer et al., Blood, 119: 2709-2720 (2012); Kalos et al., Sci Transl Med, 3(95):95ra73 (2011); Clinical Trial Study Record Nos.: NCT02315612; NCT01822652.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3(12): 668-681 (2006).

Thus in some embodiments, the provided method further involves administering a lymphodepleting therapy to the subject. In some embodiments, the method involves administering the lymphodepleting therapy to the subject prior to the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the administration of the dose of cells. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the fludarabine can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells.

In one exemplary dosage regime, prior to receiving the first dose, subjects receive an agent such as an inhibitor of microglia or one or more activity thereof 30 days before the administration of cells and an lymphodepleting preconditioning chemotherapy of cyclophosphamide and fludarabine (CY/FLU), which is administered at least two days before the first dose of CAR-expressing cells and generally no more than 7 days before administration of cells. In some cases, for example, cyclophosphamide is given from 24 to 27 days after the administration of the agent such as the microglia inhibitor. After preconditioning treatment, subjects are administered the dose of CAR-expressing T cells as described above.

In some embodiments, the administration of the preconditioning agent prior to infusion of the dose of cells improves an outcome of the treatment. For example, in some aspects, preconditioning improves the efficacy of treatment with the dose or increases the persistence of the recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In some embodiments, the administration of the preconditioning agent prior to infusion of the dose of cells improves an outcome of the treatment such as by improving the efficacy of treatment with the dose or increases the persistence of the recombinant receptor-expressing cells (e.g., CAR-expressing cells, such as CAR-expressing T cells) in the subject. Therefore, in some embodiments, the dose of preconditioning agent given in the method which is a combination therapy with the agent, e.g., the inhibitor of microglia inhibitor, and cell therapy is higher than the dose given in the method without the agent such as without the microglia inhibitor.

### III. T Cell Therapy and Engineered Cells

In some embodiments, the methods for ameliorating or reducing toxicity in a subject are associated with the administration of a cell therapy, such as for the treatment of diseases or conditions including various tumors. In some embodiments, the T cell therapy for use in accord with the provided combination therapy methods includes administering engineered cells expressing recombinant receptors designed to recognize and/or specifically bind to molecules associated with the disease or condition and result in a response, such as an immune response against such molecules upon binding to such molecules. The receptors may include chimeric receptors, e.g., chimeric antigen receptors (CARs), and other transgenic antigen receptors including transgenic T cell receptors (TCRs).

In some embodiments, the cells contain or are engineered to contain an engineered receptor, e.g., an engineered antigen receptor, such as a chimeric antigen receptor (CAR), or a T cell receptor (TCR). Also provided are populations of such cells, compositions containing such cells and/or enriched for such cells, such as in which cells of a certain type such as T cells or CD8⁺ or CD4⁺ cells are enriched or selected. Among the compositions are pharmaceutical compositions and formulations for administration, such as for adoptive cell therapy. Also provided are therapeutic methods for administering the cells and compositions to subjects, e.g., patients.

Thus, in some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

A. Chimeric Antigen Receptors (CARs)

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

1. Chimeric Antigen Receptors (CARS)

Exemplary antigen receptors, including CARs, and methods for engineering and introducing such receptors into cells, include those described, for example, in international patent application publication numbers WO2000/14257, WO2013/126726, WO2012/129514, WO2014/031687, WO2013/166321, WO2013/071154, WO2013/123061 U.S. patent application publication numbers US2002/131960, US2013/287748, US2013/0149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov., 3(4): 388-398 (2013); Davila et al. PLoS ONE 8(4): e61338 (2013); Turtle et al., Curr. Opin. Immunol., 24(5): 633-39 (2012); Wu et al., Cancer, 18(2): 160-75 (2012). In some aspects, the antigen receptors include a CAR as described in U.S. Pat. No. 7,446,190, and those described in International Patent Application Publication No.: WO2014/055668 A1. Examples of the CARs include CARs as disclosed in any of the aforementioned publications, such as WO2014/031687, U.S. Pat. Nos. 8,339,645, 7,446,179, US 2013/0149337, U.S. Pat. Nos. 7,446,190, 8,389,282, Kochenderfer et al., Nature Reviews Clinical Oncology, 10, 267-276 (2013); Wang et al., J. Immunother. 35(9): 689-701 (2012); and Brentjens et al., Sci Transl Med., 5(177) (2013). See also WO2014031687, U.S. Pat. Nos. 8,339,645, 7,446,179, US 2013/0149337, U.S. Pat. Nos. 7,446,190, and 8,389,282. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens targeted by the receptors in some embodiments include αvβ6 integrin (avb6 integrin), B cell maturation antigen (BCMA), B7-H6, carbonic anhydrase 9 (CA9, also known as CAIX or G250), a cancer-testis antigen, cancer/testis antigen 1B (CTAG, also known as NY-ESO-1 and LAGE-2), C-C Motif Chemokine Ligand 1 (CCL-1), orphan tyrosine kinase receptor ROR1, tEGFR, Her2, L1-CAM, CD19, CD20, CD22, CD44v6, CD44v7/8, CD123, CD138, CD171, mesothelin, CEA, and hepatitis B surface antigen, anti-folate receptor, CD23, CD24, CD30, CD33, CD38, CD44, EGFR, type III epidermal growth factor receptor mutation (EGFR vIII), epithelial glycoprotein 2 (EGP-2), EGP-4, epithelial glycoprotein 40 (EPG-40), ephrinB2, ephrine receptor A2 (EPHa2), ErbB2, 3, or 4, estrogen receptor, Fc receptor like 5 (FCRL5; also known as Fc receptor homolog 5 or FCRH5), fetal acetylcholine receptor (fetal AchR), a folate binding protein (FBP), folate receptor alpha, fetal acethycholine receptor, ganglioside GD2, O-acetylated GD2 (OGD2), ganglioside GD3, G Protein Coupled Receptor 5D (GPCR5D)Her2/neu (receptor tyrosine kinase erbB2), Her3 (erb-B3), Her4 (erb-B4), erbB dimers, Human high molecular weight-melanoma-associated antigen (HMW-MAA), hepatitis B surface antigen, Human leukocyte antigen A1 (HLA-AI), Human leukocyte antigen A2 (HLA-A2), IL-22 receptor alpha (IL-22R-alpha), IL-13 receptor alpha 2 (IL-13R-alpha2), kinase insert domain receptor (kdr), kappa light chain, CE7 epitope of L1-CAM, Leucine Rich Repeat Containing 8 Family Member A (LRRC8A), Lewis Y, L1-cell adhesion molecule (L1CAM), Melanoma-associated antigen (MAGE)-A1, MAGE-A3, MAGE-A6, mesothelin, c-Met, murine cytomegalovirus (CMV), mucin 1 (MUC1), MUC16, preferentially expressed antigen of melanoma (PRAME), progesterone receptor, a prostate specific antigen, prostate stem cell antigen (PSCA), natural killer group 2 member D (NKG2D) Ligands, melan A (MART-1), neural cell adhesion molecule (NCAM), glycoprotein 100 (gp100), oncofetal antigen, prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGF-R2), carcinoembryonic antigen (CEA), prostate specific antigen, PSMA, Her2/neu, estrogen receptor, progesterone receptor, ephrinB2, CD123, c-Met, GD-2, and MAGE A3, CE7, Wilms Tumor 1 (WT-1), a cyclin, such as cyclin A1 (CCNA1), cyclin A2, and/or biotinylated molecules, and/or molecules expressed by HIV, HCV, HBV or other pathogens.

In some embodiments, the CAR binds a pathogen-specific antigen. In some embodiments, the CAR is specific for viral antigens (such as HIV, HCV, HBV, etc.), bacterial antigens, and/or parasitic antigens. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the antigen is a pathogen-specific antigen. In some embodiments, the antigen is a viral antigen (such as a viral antigen from HIV, HCV, HBV, etc.), bacterial antigens, and/or parasitic antigens.

In some embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. Clin. Cancer Res., 19:3153 (2013), international patent application publication number WO2014/031687, U.S. Pat. No. 8,822,647 or published app. No. US2014/0271635.

In some embodiments, the constant region or portion is of a human IgG, such as IgG4 or IgG1. In some embodiments, the spacer has the sequence ESKYGPPCPPCP (set forth in SEQ ID NO: 1), and is encoded by the sequence set forth in SEQ ID NO: 2. In some embodiments, the spacer has the sequence set forth in SEQ ID NO: 3. In some embodiments, the spacer has the sequence set forth in SEQ ID NO: 4. In some embodiments, the constant region or portion is of IgD. In some embodiments, the spacer has the sequence set forth in SEQ ID NO: 5. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 1, 3, 4 or 5.

This antigen recognition domain generally is linked to one or more intracellular signaling components, such as signaling components that mimic activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86, CD 134, CD137, CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CD8, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014/031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7.

An exemplary T2A linker sequence comprises the sequence of amino acids set forth in SEQ ID NO: 6, 18, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 18. Other exemplary 2A sequences include the foot-and-mouth disease virus (F2A, e.g., SEQ ID NO: 22), equine rhinitis A virus (E2A, e.g., SEQ ID NO: 21), and porcine teschovirus-1 (P2A, e.g., SEQ ID NO:19 or 20)

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P01747.1) or variant thereof, such as a transmembrane domain that comprises the sequence of amino acids set forth in SEQ ID NO: 8 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8; in some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular domain comprises an intracellular costimulatory signaling domain of 4-1BB (e.g. (Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular signaling domain of the recombinant receptor, e.g. the CAR, comprises a human CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190 or 8,911,993. For example, in some embodiments, the intracellular signaling domain comprises the sequence of amino acids as set forth in SEQ ID NO: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13, 14 or 15.

In some aspects, the spacer contains only a hinge region of an IgG, such as only a hinge of IgG4 or IgG1, such as the hinge only spacer set forth in SEQ ID NO: 1. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 4. In some embodiments, the spacer is an Ig hinge, e.g., an IgG4 hinge, linked to a CH3 domain only, such as set forth in SEQ ID NO: 3. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, a single promoter may direct expression of an RNA that contains, in a single open reading frame (ORF), two or three genes (e.g. encoding the molecule involved in modulating a metabolic pathway and encoding the recombinant receptor) separated from one another by sequences encoding a self-cleavage peptide (e.g., 2A sequences) or a protease recognition site (e.g., furin). In some embodiments, nucleic acid molecules encoding such CAR constructs further includes a sequence encoding a T2A ribosomal skip element and/or a tEGFR sequence, e.g., downstream of the sequence encoding the CAR. The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein, without limitation, 2A sequences from the foot-and-mouth disease virus (F2A, e.g., SEQ ID NO: 22), equine rhinitis A virus (E2A, e.g., SEQ ID NO: 21), Thosea asigna virus (T2A, e.g., SEQ ID NO: 6 or 18), and porcine teschovirus-1 (P2A, e.g., SEQ ID NO: 19) as described in U.S. Patent Publication No. 20070116690. In some embodiments, the sequence encodes a self-cleavage peptide, e.g., a T2A ribosomal skip element set forth in SEQ ID NO: 6 or 18, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 18. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a self-cleavage peptide (e.g., T2A ribosome switch) to express two proteins from the same construct), which then can be used as a marker to detect such cells (see e.g. U.S. Pat. No. 8,802,374). In some embodiments, the sequence encodes an tEGFR sequence set forth in SEQ ID NO: 7, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

B. TCRs

In some embodiments, engineered cells, such as T cells, are provided that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or Cβ, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g., CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified by a skilled artisan. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using computer prediction models known to those of skill in the art. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known to those of skill in the art. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007)

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known to those of skill in the art, See e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)5-P- wherein P is proline, G is glycine and S is serine (SEQ ID NO: 16). In some embodiments, the linker has the sequence

(SEQ ID NO: 17)
GSADDAKKDAAKKDGKS

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λ610, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other promoters known to a skilled artisan also are contemplated.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

C. Multi-Targeting

In some embodiments, the cells and methods include multi-targeting strategies, such as expression of two or more genetically engineered receptors on the cell, each recognizing the same of a different antigen and typically each including a different intracellular signaling component. Such multi-targeting strategies are described, for example, in International Patent Application, Publication No.: WO 2014/055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR or TCR) which is capable of inducing an activating signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR or TCR) is capable of inducing an activating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF-κB and/or AP-1, and/or induction of gene expression of factors such as cytokines, proliferation, and/or survival.

In some embodiments, the first and/or second receptor includes intracellular signaling domains of costimulatory receptors such as CD28, CD137 (4-1 BB), OX40, and/or ICOS. In some embodiments, the first and second receptor include an intracellular signaling domain of a costimulatory receptor that are different. In one embodiment, the first receptor contains a CD28 costimulatory signaling region and the second receptor contain a 4-1BB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

D. Vectors and Methods for Genetic Engineering

Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. Gene Therapy doi: 10.1038/gt.2014.25 (2014); Carlens et al. Exp Hematol., 28(10): 1137-46 (2000); Alonso-Camino et al. Mol Ther Nucl Acids, 2, e93 (2013); Park et al., Trends Biotechnol., November 29(11): 550-557 (2011).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman, BioTechniques, 7:980-990 (1989); Miller, A. D. Human Gene Therapy, 1:5-14 (1990); Scarpa et al. Virology, 180:849-852 (1991); Burns et al. Proc. Natl. Acad. Sci. USA, 90:8033-8037 (1993); and Boris-Lawrie and Temin, Cur. Opin. Genet. Develop., 3:102-109 (1993).

Methods of lentiviral transduction are known. Exemplary methods are described in, e.g., Wang et al., J. Immunother., 35(9): 689-701 (2012); Cooper et al. Blood. 101:1637-1644 (2003); Verhoeyen et al., Methods Mol Biol., 506: 97-114 (2009); and Cavalieri et al., Blood., 102(2): 497-505 (2003).

In some embodiments, recombinant nucleic acids are transferred into T cells via electroporation (see, e.g., Chicaybam et al, PLoS ONE 8(3): e60298 (2013) and Van Tedeloo et al. Gene Therapy 7(16): 1431-1437 (2000)). In some embodiments, recombinant nucleic acids are transferred into T cells via transposition (see, e.g., Manuri et al. Hum Gene Ther 21(4): 427-437 (2010); Sharma et al. Molec Ther Nucl Acids 2, e74 (2013); and Huang et al. Methods Mol Biol 506: 115-126 (2009)). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g., with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g., via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, Methods Mol Biol. 907:645-66 (2012); or Barrett et al., Chimeric Antigen Receptor Therapy for Cancer Annual Review of Medicine, Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

E. Cells and Preparation of Cells for Genetic Engineering

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura al. Blood. 1:72-82 (2012); Wang et al. J Immunother. 35(9):689-701 (2012). In some embodiments, combining TCM-enriched CD8+ T cells and CD4+ T cells further enhances efficacy.

In embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L- and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 2011/0003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. J Immunother. 35(9): 651-660 (2012), Terakura et al. Blood. 1:72-82 (2012), and Wang et al. J Immunother. 35(9):689-701 (2012).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. Lab Chip 10, 1567-1573 (2010); and Godin et al. J Biophoton. 1(5):355-376 (2008). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3. In some embodiments, the stimulating conditions include one or more agent, e.g. ligand, which is capable of stimulating a costimulatory receptor, e.g., anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. J Immunother. 35(9): 651-660 (2012), Terakura et al. Blood. 1:72-82 (2012), and/or Wang et al. J Immunother. 35(9):689-701 (2012).

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### IV. Exemplary Treatment Outcomes and Methods of Assessing Same

In some embodiments of the methods, compositions, combinations, kits and uses provided herein, the provided combination therapy results in one or more treatment outcomes, such as a feature associated with any one or more of the parameters associated with the therapy or treatment, as described below. In some embodiments, the provided methods reduce or ameliorate a toxic outcome in a subject. In some embodiments, the combination therapy can further include one or more screening steps to identify subjects for treatment with the combination therapy and/or continuing the combination therapy, and/or a step for assessment of treatment outcomes and/or monitoring treatment outcomes. In some embodiments, the step for assessment of treatment outcomes can include steps to evaluate and/or to monitor toxicity or and/or to evaluate or monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the combination therapy provided herein.

In some embodiments, a toxic outcome or symptom in the subject is reduced or ameliorated compared to a method in which the therapeutic agent, e.g. cell therapy, is administered to the subject in the absence of the agent, e.g. inhibitor. For example, the toxic outcome or symptom is associated with neurotoxicity or cytokine release syndrome (CRS), which optionally is severe neurotoxicity or severe CRS. In some embodiments, the toxic outcome or symptom in the subject at up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent, e.g. cell therapy, is not detectable or is reduced as compared to a method in which the therapeutic agent, e.g. cell therapy, is administered to the subject in the absence of the agent, e.g. inhibitor. In some aspects, the toxic outcome or symptom is reduced by greater than 50%, 60%, 70%, 80%, 90% or more. In some embodiments, the one or more effects are transient and/or are reversible upon discontinued administration of the agent, e.g., inhibitor.

In some embodiments, the administration of the therapeutic agent, e.g. cell therapy, does not induce cerebral edema in the subject or based on clinical data, a majority of subjects so treated do not exhibit a cerebral edema after the administration of the cell therapy.

In some embodiments, the toxicity ameliorated includes therapy-induced neuroinflammation. In some embodiments, in the provided combination therapy, neurotoxicity or severe neurotoxicity in the subject is not induced, such as grade 3 or higher neurotoxicity in the subject, grade 2 or higher neurotoxicity in the subject, or grade 1 or higher neurotoxicity in the subject is not induced. In some embodiments, the provided combination therapy, based on clinical data, does not induce neurotoxcity or does not induce severe neurotoxicity in a majority of subjects so treated, or based on clinical data, administration of the combination therapy does not result in a toxic outcome or symptom of neurotoxicity greater than grade 3, greater than grade 2 or greater than grade 1 in a majority of the subjects to treated.

In some embodiments, the toxicity ameliorated includes therapy-induced cytokine release syndrome (CRS). In some embodiments, the administration of the combination therapy does not induce CRS in the subject or does not induce severe CRS in the subject; the administration of the combination therapy does not induce grade 3 or higher CRS in the subject, does not induce grade 2 or higher CRS in the subject or does not induce grade 1 or higher CRS in the subject; based on clinical data, administration of the combination therapy does not induce CRS or does not induce severe CRS in a majority of subjects so treated; or based on clinical data, administration of the combination therapy does not result in a toxic outcome or symptom of CRS greater than grade 3, greater than grade 2 or greater than grade 1 in a majority of the subjects to treated.

In some embodiments, the methods also do not effect efficacy of the therapeutic agent, e.g., cell therapy, in the subject, such that the therapeutic agent exhibits the same or similar efficacy when administered in the presence of the agent as compared to administration of the therapeutic agent in the absence of the agent. In some embodiments, tumor burden is reduced in the subject and/or the subject responds to the therapeutic agent, such as by an objective response rate, e.g. partial response or complete response. In some embodiments, the persistence, expansion, and/or presence of recombinant receptor-expressing, e.g., CAR-expressing, cells in the subject following administration of the dose of cells in the method with the agent such as the microglia inhibitor is greater as compared to that achieved via a method without the agent such as without the microglia inhibitor.

In some embodiments, the administration of the inhibitor allows a higher dose of lymphodepleting therapy to be administered to the subject as compared to a method in which the dose of cells expressing the recombinant receptor is administered to the subject in the absence of the inhibitor. In some embodiments, the administration of the inhibitor allows a higher dose of cell therapy to be administered to the subject as compared to a method in which the dose of cells expressing the recombinant receptor is administered to the subject in the absence of the inhibitor.

A. Inhibitor Activity or Function

In some embodiments, the provided methods involved administration of an agent such as an anti-inflammatory agent or an anti-oxidative stress agent such as an agent capable of preventing, blocking, or reducing one or more microglial cell activity or function such as an inflammatory activity thereof or capable of promoting an anti-inflammatory or protective function thereof. In some embodiments, the administration of the agent reduces the number of microglial cells by greater than 20%, greater than 30%, greater than 40% or greater than 50%, greater than 60%, greater than 70%, greater than 80%, greater than 90%, greater than 95% or greater than 99% compared to at a time just prior to initiation of the administration of the agent.

In some embodiments, the method alters the level of a serum or blood biomarker of CSF1R inhibition in the subject such as an increase in plasma CSF-1, an increase in a level of a serum enzyme or a decrease in CD14dim/CD16+ nonclassical monocytes. In some aspects, the serum enzyme is alanine aminotransferase (ALT), AST, creatine kinase (CK) or LDH. Colony stimulating factor-1 (CSF-1), also termed macrophage colony stimulating factor (M-CSF), signals through its receptor CSF-1 R to regulate the differentiation, proliferation, recruitment and survival of macrophages. Therefore, in some embodiments, the method results in an alteration in the number of macrophages or myeloid cells in the blood. In some aspects, the biological sample is a bodily fluid or a tissue. In some cases, the bodily fluid includes whole blood, serum or plasma.

In some aspects, detecting the biomarker includes performing an in vitro assay. In some embodiments, the in vitro assay is an immunoassay, an aptamer-based assay, a histological or cytological assay, or an mRNA expression level assay. In some embodiments, the parameter or parameters for one or more of each of the one or more biomarkers are detected by an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay.

In some embodiments, the parameter for at least one of the one or more biomarkers is determined using a binding reagent that specifically binds to at least one biomarker. In some cases, the binding reagent is an antibody or antigen-binding fragment thereof, an aptamer or a nucleic acid probe.

In some embodiments, the activity of the agent is determined by assessing biomarkers indicative of inflammation, inflammatory cytokines, microglia activation or particular effect thereof, such as by assessing a factor including serum cytokine such as TNF-α, IL-6, and IL-1β such as in the brain or CNS or blood, or by imaging microglia in the brain using positron emission tomography (PET) and magnetic resonance (MR) imaging.

In some aspects, the activity of the agent can be determined by imaging to detect microglial cell and/or the presence of neuroinflammation in the brain. Imaging, such as imaging of microglia, may be accomplished by using ligands that bind to translocator protein-18 kDa (TSPO). Exemplary ligands that bind to TSPO for use in neuroimaging of microglia include ¹¹C PBR28, ¹¹C isoquinoline (R)-PK11195, ¹¹C vinpocetine, ¹¹C DAA1106 as discussed in Lautner et al. Int J Alzheimers Dis. 2011: 939426 (2011). In some embodiments, agents for imaging brain microglia activity in vivo include the use of iron oxide nanoparticles and ultra-small super paramagnetic particles that are phagocytosed (Venneti et al., Glia 61(1):10-23 (2013)).

In some embodiments, neuroimaging and biomarker assessments that reveal a lack of microglia activation or lack or reduction of inflammatory function or response thereof indicate reduced or ameliorated neurotoxicity, confirming the effectiveness of the method using the inhibitor compared to a method in which the cell therapy is administered to the subject in the absence of the inhibitor. In some embodiments, assessment or monitoring of neurotoxicity biomarkers is performed at the time of the administration of the cell therapy and/or after the administration of the cell therapy.

B. Ameliorating Neurotoxicity

In some embodiments, the therapy-induced toxic outcome or symptom is associated with neurotoxicity. In some embodiments, symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute—Common Toxicity Criteria version 4.03 (NCI-CTCAE v4.03). In some cases, the neurotoxicity is severe neurotoxicity and/or the neurotoxicity is a grade 3 or higher neurotoxicity. In some embodiments, the toxic outcome or symptom is associated with grade 3, grade 4 or grade 5 neurotoxicity.

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 19 days. In some cases, recovery of neurologic changes occurs after other symptoms of sCRS have resolved. In some embodiments, time or degree of resolution of neurologic changes is not hastened by treatment with anti-IL-6 and/or steroid(s).

As used herein, a subject is deemed to develop “severe neurotoxicity” in response to or secondary to administration of a cell therapy or dose of cells thereof, if, following administration, the subject displays symptoms that limit self-care (e.g. bathing, dressing and undressing, feeding, using the toilet, taking medications) from among: 1) symptoms of peripheral motor neuropathy, including inflammation or degeneration of the peripheral motor nerves; 2) symptoms of peripheral sensory neuropathy, including inflammation or degeneration of the peripheral sensory nerves, dysesthesia, such as distortion of sensory perception, resulting in an abnormal and unpleasant sensation, neuralgia, such as intense painful sensation along a nerve or a group of nerves, and/or paresthesia, such as functional disturbances of sensory neurons resulting in abnormal cutaneous sensations of tingling, numbness, pressure, cold and warmth in the absence of stimulus. In some embodiments, severe neurotoxicity includes neurotoxicity with a grade of 3 or greater, such as set forth in Table 2.

TABLE 2
Exemplary Grading Criteria for neurotoxicity

| Grade | Description of Symptoms |
|---|---|
| 1 | Mild or asymptomatic symptoms |
| Asymptomatic or Mild | |
| 2 | Presence of symptoms that limit instrumental |
| Moderate | activities of daily living (ADL), |
| | such as preparing meals, shopping for |
| | groceries or clothes, using the |
| | telephone, managing money |
| 3 | Presence of symptoms that limit self-care |
| Severe | ADL, such as bathing, dressing and |
| | undressing, feeding self, using the toilet, |
| | taking medications |
| 4 | Symptoms that are life-threatening, |
| Life-threatening | requiring urgent intervention |
| 5 | Death |
| Fatal | |

In some embodiments, the methods reduce symptoms associated with CNS-outcomes or neurotoxicity compared to other methods. For example, subjects treated according to the present methods may lack detectable and/or have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects treated by other methods in which the inhibitor is not administered. In some embodiments, subjects treated according to the present methods may have reduced symptoms associated with peripheral motor neuropathy, peripheral sensory neuropathy, dysethesia, neuralgia or paresthesia.

In some embodiments, the methods reduce outcomes associated with neurotoxicity including damages to the nervous system and/or brain, such as the death of neurons. In some aspects, the methods reduce the level of factors associated with neurotoxicity such as beta amyloid (Aβ), glutamate, and oxygen radicals. In some embodiments, the symptom or outcome is cerebral edema which co-presents with neurotoxicity. In some cases, the cerebral edema involves alterations in blood brain barrier function and or tight junction integrity.

In some embodiments, administration of the agent reduces symptoms associated with neurotoxicity compared to other methods. For example, subjects treated with the inhibitor may have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects who do not receive the agent, or receive the agent at a time when physical symptoms of neurotoxicity have manifested in the subject. In some embodiments, subjects treated with the agent according to the present methods may have reduced symptoms associated with peripheral motor neuropathy, peripheral sensory neuropathy, dysethesia, neuralgia or paresthesia.

The toxic outcome or symptoms is one or more of confusion, delirium, expressive aphasia, obtundation, myoclonus, lethargy, altered mental status, convulsions, seizure-like activity, seizures (optionally as confirmed by electroencephalogram [EEG]), cerebral edema, elevated levels of beta amyloid (Aβ), elevated levels of glutamate, and elevated levels of oxygen radicals, encephalopathy, dysphasia, tremor, choreoathetosis, symptoms that limit self-care, symptoms of peripheral motor neuropathy, symptoms of peripheral sensory neuropathy and combinations thereof.

In some embodiments, a toxic outcome or symptom of neurotoxicity in the subject at day up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent, e.g. cell therapy, is not detectable or is reduced as compared to a method in which the cell therapy is administered to the subject in the absence of the agent. In some aspects, the toxic outcome or symptom of neurotoxicity is reduced by greater than 50%, 60%, 70%, 80%, 90% or more.

In some aspects, the physical signs or symptoms associated with toxicity include e.g., severe neurotoxicity, include confusion, delirium, expressive aphasia, obtundation, myoclonus, lethargy, altered mental status, convulsions, seizure-like activity, seizures (such as confirmed by electroencephalogram [EEG]), encephalopathy, dysphasia, tremor, choreoathetosis, symptoms that limit self-care, symptoms of peripheral motor neuropathy, symptoms of peripheral sensory neuropathy or combinations thereof. In some cases, the physical signs or symptoms associated with toxicity, e.g., severe neurotoxicity, are associated with grade 3, grade 4 or grade 5 neurotoxicity. In some embodiments, the physical signs or symptoms associated with toxicity, e.g., severe neurotoxicity, manifest greater than or greater than about or about 5 days after cell therapy, 6 days after cell therapy or 7 days after cell therapy.

In some embodiments, the method ameliorates neurotoxicity, e.g., severe neurotoxicity and/or reduces the physical signs or symptoms of severe neurotoxicity compared to a subject in which severe neurotoxicity is treated after the subject exhibits a physical sign or symptom of neurotoxicity and/or compared to a subject in which severe neurotoxicity is treated greater than 5 days, greater than 6 days or greater than 7 days after administration of the cell therapy. In some cases, the treated subject does not exhibit grade 3 or higher neurotoxicity or a majority of treated subjects do not exhibit grade 3 or higher neurotoxicity.

C. Ameliorating Cytokine Release Syndrome

In some embodiments, the toxic outcome or symptom is associated with cytokine-release syndrome (CRS). In some embodiments, the CRS is severe CRS and/or the CRS is grade 3 or higher CRS. In some cases, the toxic outcome or symptom is one or more of fever, hypotension, hypoxia, neurologic disturbances, or elevated serum level of an inflammatory cytokine or C reactive protein (CRP). In some embodiments, the toxic outcome or symptom of CRS in the subject at day up to or up to about day 7, 8, 9, 10, 11, 12, 13, 14, 15, 16, 17, 18, 19, 20, 21, 22, 23, 24, 25, 26, 27, 28, 29 or 30 following initiation of administration of the cell therapy is not detectable or is reduced as compared to a method in which the cell therapy is administered to the subject in the absence of the agent. In some embodiments, CRS is reduced by greater than 50%, 60%, 70%, 80%, 90% or more.

In some aspects, the toxic outcome of a therapy, such as a cell therapy, is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013); Grupp et al., N. Engl. J. Med. 368, 1509-1518 (2013); and Kochenderfer et al., Blood 119, 2709-2720 (2012); Xu et al., Cancer Letters 343 (2014) 172-78.

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure.

Outcomes, signs and symptoms of CRS are known and include those described herein. In some embodiments, where a particular dosage regimen or administration effects or does not effect a given CRS-associated outcome, sign, or symptom, particular outcomes, signs, and symptoms and/or quantities or degrees thereof may be specified.

In the context of administering CAR-expressing cells, CRS, such as severe CRS, typically occurs 6-20 days after infusion of cells that express a CAR. See Xu et al., Cancer Letters 343 (2014) 172-78. In some cases, CRS occurs less than 6 days or more than 20 days after CAR T cell infusion. The incidence and timing of CRS may be related to baseline cytokine levels or tumor burden at the time of infusion. Commonly, CRS involves elevated serum levels of interferon (IFN)-γ, tumor necrosis factor (TNF)-α, and/or interleukin (IL)-2. Other cytokines that may be rapidly induced in CRS are IL-1β, IL-6, IL-8, and IL-10.

CRS criteria that appear to correlate with the onset of CRS to predict which patients are more likely to be at risk for developing sCRS have been developed (see Davilla et al. Science translational medicine. 2014; 6(224):224ra25). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. Other guidelines on the diagnosis and management of CRS are known (see e.g., Lee et al, Blood. 2014; 124(2):188-95). In some embodiments, the criteria reflective of CRS grade are those detailed in Table 3 below.

TABLE 3
Exemplary Grading Criteria for CRS

| Grade | Description of Symptoms |
|---|---|
| 1 | Not life-threatening, require only |
| Mild | symptomatic treatment such as antipyretics |
| | and anti-emetics (e.g., fever, nausea, |
| | fatigue, headache, myalgias, malaise) |
| 2 | Require and respond to moderate intervention: |
| Moderate | Oxygen requirement < 40%, or |
| | Hypotension responsive to fluids or low |
| | dose of a single vasopressor, or |
| | Grade 2 organ toxicity (by CTCAE v4.0) |
| 3 | Require and respond to aggressive intervention: |
| Severe | Oxygen requirement ≥ 40%, or |
| | Hypotension requiring high dose of a |
| | single vasopressor (e.g., |
| | norepinephrine ≥ 20 μg/kg/min, |
| | dopamine ≥ 10 μg/kg/min, |
| | phenylephrine ≥ 200 μg/kg/min, |
| | or epinephrine ≥ 10 μg/kg/min), or |
| | Hypotension requiring multiple vasopressors |
| | (e.g., vasopressin + one of the above agents, |
| | or combination vasopressors equivalent |
| | to ≥ 20 μg/kg/min norepinephrine), or |
| | Grade 3 organ toxicity or Grade 4 transaminitis |
| | (by CTCAE v4.0) |
| 4 | Life-threatening: |
| Life-threatening | Requirement for ventilator support, or |
| | Grade 4 organ toxicity (excluding transaminitis) |
| 5 | Death |
| Fatal | |

As used herein, a subject is deemed to develop “severe CRS” (“sCRS”) in response to or secondary to administration of a cell therapy or dose of cells thereof, if, following administration, the subject displays: (1) fever of at least 38 degrees Celsius for at least three days; (2) cytokine elevation that includes either (a) a max fold change of at least 75 for at least two of the following group of seven cytokines compared to the level immediately following the administration: interferon gamma (IFNγ), GM-CSF, IL-6, IL-10, Flt-3L, fracktalkine, and IL-5 and/or (b) a max fold change of at least 250 for at least one of the following group of seven cytokines compared to the level immediately following the administration: interferon gamma (IFNγ), GM-CSF, IL-6, IL-10, Flt-3L, fracktalkine, and IL-5; and (c) at least one clinical sign of toxicity such as hypotension (requiring at least one intravenous vasoactive pressor) or hypoxia (PO₂<90%) or one or more neurologic disorder(s) (including mental status changes, obtundation, and/or seizures). In some embodiments, severe CRS includes CRS with a grade of 3 or greater.

In some embodiments, outcomes associated with severe CRS or grade 3 CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures).

In some embodiments, severe CRS encompasses a combination of (1) persistent fever (fever of at least 38 degrees Celsius for at least three days) and (2) a serum level of CRP of at least at or about 20 mg/dL. In some embodiments, severe CRS encompasses hypotension requiring the use of two or more vasopressors or respiratory failure requiring mechanical ventilation.

In some embodiments, the subject exhibits a fever, and in some aspects is treated at a time at which the subject exhibits such fever and/or exhibits or has exhibited the fever for a particular period of time.

In some embodiments, the fever in the subject is characterized as a body temperature of the subject that is (or is measured at) at or above a certain threshold temperature or level. In some aspects, the threshold temperature is that associated with at least a low-grade fever, with at least a moderate fever, and/or with at least a high-grade fever. In some embodiments, the threshold temperature is a particular temperature or range. For example, the threshold temperature may be at or about 38, 39, 40, 41, or 42 degrees Celsius, and/or may be a range of at or about 38 degrees Celsius to at or about 39 degrees Celsius, a range of at or about 39 degrees Celsius to at or about 40 degrees Celsius, a range of at or about 40 degrees Celsius to at or about 41 degrees, or a range of at or about 41 degrees Celsius to at or about 42 degrees Celsius.

In some embodiments, the fever is a sustained fever; in some aspects, the subject is treated at a time at which a subject has been determined to have a sustained fever, such as within one, two, three, four, five six, or fewer hours of such determination or of the first such determination following the initial therapy having the potential to induce the toxicity, such as the disease-targeted therapy.

In some embodiments, the subject has, and/or is determined to or considered to have, a sustained fever if he or she exhibits a fever at or above the relevant threshold temperature, and where the fever or body temperature of the subject does not fluctuate by about, or by more than about, 1° C., and generally does not fluctuate by about, or by more than about, 0.5° C., 0.4° C., 0.3° C., or 0.2° C. Such absence of fluctuation above or at a certain amount generally is measured over a given period of time (such as over a 24-hour, 12-hour, 8-hour, 6-hour, 3-hour, or 1-hour period of time, which may be measured from the first sign of fever or the first temperature above the indicated threshold). For example, in some embodiments, a subject is considered to or is determined to exhibit sustained fever if he or she exhibits a fever of at least at or about 38 or 39 degrees Celsius, which does not fluctuate in temperature by more than at or about 0.5° C., 0.4° C., 0.3° C., or 0.2° C., over a period of 6 hours, over a period of 8 hours, or over a period of 12 hours, or over a period of 24 hours.

In some embodiments, the subject has, and/or is determined to or considered to have, a sustained fever if he or she exhibits a fever at or above the relevant threshold temperature, and where the fever or body temperature of the subject is not reduced, or is not reduced by or by more than a specified amount (e.g., by more than 1° C., and generally does not fluctuate by about, or by more than about, 0.5° C., 0.4° C., 0.3° C., or 0.2° C.), following a specified treatment, such as a treatment designed to reduce fever such as an antipyretic. An antipyretic may include any agent, e.g., compound, composition, or ingredient, that reduces fever, such as one of any number of agents known to have antipyretic effects, such as NSAIDs (such as ibuprofen, naproxen, ketoprofen, and nimesulide), salicylates, such as aspirin, choline salicylate, magnesium salicylate, and sodium salicylate, paracetamol, acetaminophen, Metamizole, Nabumetone, Phenaxone, antipyrine, febrifuges. In some embodiments, the antipyretic is acetaminophen. In some embodiments, it is or comprises ibuprofen or aspirin. For example, a subject is considered to have a sustained fever if he or she exhibits or is determined to exhibit a fever of at least at or about 38 or 39 degrees Celsius, which is not reduced by or is not reduced by more than at or about 0.5° C., 0.4° C., 0.3° C., or 0.2° C., or by at or about 1%, 2%, 3%, 4%, or 5%, over a period of 6 hours, over a period of 8 hours, or over a period of 12 hours, or over a period of 24 hours, even following treatment with the antipyretic such as tylenol. In some embodiments, the dosage of the antipyretic is a dosage ordinarily effective in such as subject to reduce fever or fever of a particular type such as fever associated with a bacterial or viral infection, e.g., a localized or systemic infection.

In some embodiments, the amelioration of CRS is determined by assessing biomarkers indicative of CRS including serum factors and inflammatory cytokines such as IFNγ, GM-CSF, TNFα, IL-6, IL-10, IL-1β, IL-8, IL-2, MIP-1, Flt-3L, fracktalkine, and IL-5. In some embodiments, assessment or monitoring of CRS biomarkers is performed at the time of the administration of the cell therapy and/or after the administration of the cell therapy.

In some aspects, detecting the biomarker includes performing an in vitro assay. In some embodiments, the in vitro assay is an immunoassay, an aptamer-based assay, a histological or cytological assay, or an mRNA expression level assay. In some embodiments, the parameter or parameters for one or more of each of the one or more biomarkers are detected by an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay.

In some embodiments, the parameter for at least one of the one or more biomarkers is determined using a binding reagent that specifically binds to at least one biomarker. In some cases, the binding reagent is an antibody or antigen-binding fragment thereof, an aptamer or a nucleic acid probe.

D. Enhancing Treatment

In some embodiments, the methods also affect efficacy of the cell therapy in the subject. In some embodiments, the persistence, expansion, and/or presence of recombinant receptor-expressing, e.g., CAR-expressing, cells in the subject following administration of the dose of cells in the method with the agent such as the agent that reduces a microglial cell activity or function or type thereof is greater as compared to that achieved via a method without the agent. In some embodiments, the administration of the agent decreases tumor burden, in the subject as compared to a method in which the dose of cells expressing the recombinant receptor is administered to the subject in the absence of the agent. In some embodiments, the administration of the agent decreases blast marrow in the subject as compared to a method in which the dose of cells expressing the recombinant receptor is administered to the subject in the absence of the agent.

The exposure, e.g., number of cells, indicative of expansion and/or persistence, may be stated in terms of maximum numbers of the cells to which the subject is exposed, duration of detectable cells or cells above a certain number or percentage, area under the curve for number of cells over time, and/or combinations thereof and indicators thereof. Such outcomes may be assessed using known methods, such as qPCR to detect copy number of nucleic acid encoding the recombinant receptor compared to total amount of nucleic acid or DNA in the particular sample, e.g., blood or serum, and/or flow cytometric assays detecting cells expressing the receptor generally using antibodies specific for the receptors. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor.

In some aspects, increased exposure of the subject to the cells includes increased expansion of the cells. In some embodiments, the receptor- (e.g., CAR-) expressing cells expand in the subject following administration of the first dose and/or following administration of the consecutive dose. In some aspects, the methods result in greater expansion of the cells compared with other methods, such as those involving the administration of the cells as a single dose, administration of larger first doses, administration of the consecutive dose without administering the first dose, and/or methods in which a consecutive dose is administered before or after the specified window of time or time point, such that, for example, an immune response develops prior to the administration of the first dose.

In some aspects, the method results in high in vivo proliferation of the administered cells, for example, as measured by flow cytometry. In some aspects, high peak proportions of the cells are detected. For example, in some embodiments, at a peak or maximum level following the first or consecutive administration, in the blood or disease-site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the CAR.

In some embodiments, the method results in a maximum concentration, in the blood or serum or other bodily fluid or organ or tissue of the subject, of at least 100, 500, 1000, 1500, 2000, 5000, 10,000 or 15,000 copies of or nucleic acid encoding the receptor, e.g., the CAR per microgram of DNA, or at least 0.1, 0.2, 0.3, 0.4, 0.5, 0.6, 0.7, 0.8, or 0.9 receptor-expressing, e.g., CAR,-expressing cells per total number of peripheral blood mononuclear cells (PBMCs), total number of mononuclear cells, total number of T cells, or total number of microliters. In some embodiments, the cells expressing the receptor are detected as at least 10, 20, 30, 40, 50, or 60% of total PBMCs in the blood of the subject, and/or at such a level for at least 1, 2, 3, 4, 5, 6, 7, 8, 9, 10, 11, 12, 24, 36, 48, or 52 weeks following the first or consecutive administration or for 1, 2, 3, 4, or 5, or more years following such administration.

In some aspects, the method results in at least a 2-fold, at least a 4-fold, at least a 10-fold, or at least a 20-fold increase in copies of nucleic acid encoding the recombinant receptor, e.g., CAR, per microgram of DNA, e.g., in the serum of the subject.

In some embodiments, cells expressing the receptor are detectable in the blood or serum of the subject, e.g., by a specified method, such as qPCR or flow cytometry-based detection method, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more days following administration of the first dose or after administration of the consecutive dose, for at least at or about 2, 3, 4, 5, 6, 7, 8, 9, 10, 11, 12, 13, 14, 15, 16, 17, 18, 19, 20, 21, 22, 23, or 24 or more weeks following the administration of the first dose or the consecutive dose.

In some aspects, at least about 1×10², at least about 1×10³, at least about 1×10⁴, at least about 1×10⁵, or at least about 1×10⁶ or at least about 5×10⁶ or at least about 1×10⁷ or at least about 5×10⁷ or at least about 1×10⁸ recombinant receptor-expressing, e.g., CAR-expressing cells, and/or at least 10, 25, 50, 100, 200, 300, 400, or 500, or 1000 receptor-expressing cells per microliter, e.g., at least 10 per microliter, are detectable or are present in the subject or fluid, tissue, or compartment thereof, such as in the blood, e.g., peripheral blood, or disease site thereof. In some embodiments, such a number or concentration of cells is detectable in the subject for at least about 20 days, at least about 40 days, or at least about 60 days, or at least about 3, 4, 5, 6, 7, 8, 9, 10, 11, or 12 months, or at least 2 or 3 years, following administration of the first dose or following the administration of the consecutive dose(s). Such cell numbers may be as detected by flow cytometry-based or quantitative PCR-based methods and extrapolation to total cell numbers using known methods. See, e.g., Brentjens et al., Sci Transl Med. 5(177) (2013), Park et al, Molecular Therapy 15(4):825-833 (2007), Savoldo et al., JCI 121(5):1822-1826 (2011), Davila et al. (2013) PLoS ONE 8(4):e61338, Davila et al., Oncoimmunology 1(9):1577-1583 (2012), Lamers, Blood 117:72-82 (2011), Jensen et al. Biol Blood Marrow Transplant, 16(9): 1245-1256 (2010), Brentjens et al., Blood 118(18):4817-4828 (2011).

In some aspects, the increased or prolonged expansion and/or persistence of the dose of cells in the subject administered with the agent such as the agent that reduces microglial cell activity is associated with a benefit in tumor related outcomes in the subject. In some embodiments, the tumor related outcome is selected from a decrease in tumor burden or a decrease in blast marrow in the subject. In some embodiments, the burden is decreased by or by at least at or about 10, 20, 30, 40, 50, 60, 70, 90, or 100 percent after administration. In some embodiments, disease burden, tumor size, tumor volume, tumor mass, and/or tumor load or bulk is reduced following the dose of cells by at least at or about 50, 60, 70, 80, 90% or more compared a subject that has been treated with a method that does not involve the administration of the agent.

In some embodiments, the burden of disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the mass of a solid tumor and/or the number or extent of metastases. In some aspects, survival of the subject, survival within a certain time period, extent of survival, presence or duration of event-free or symptom-free survival, or relapse-free survival, is assessed.

In some embodiments, any symptom of the disease or condition is assessed. In some embodiments, the measure of disease or condition burden is specified. In some embodiments, disease burden is low if the subject does not exhibit substantial morphologic disease or does not exhibit morphologic disease, or that exhibits less than 20% of blast cells in bone marrow, less than 15% of blast cells in bone marrow, less than 10% blast cells in bone marrow or less than 5% blast cells in bone marrow. In some embodiments, disease burden is low if the subject exhibits non-morphologic disease, such as exhibits minimal residual disease or molecularly detectable disease but does not exhibit the features associated with morphological disease as known in the art of described elsewhere, such as does not exhibit greater than 5% of blast cells in bone marrow.

### V. Articles of Manufacture and Kits

Also provided are articles of manufacture containing an agent such as an anti-inflammatory agent or anti-oxidative stress agent, or agent that reduces microglial cell activity, such as an inhibitor of microglial cell activity, e.g. CSF1R inhibitor or NRF2 pathway-modulating agent, and components for the immunotherapy, e.g., antibody or antigen binding fragment thereof or T cell therapy, e.g. engineered cells, and/or compositions thereof. In some embodiments, provided are articles of manufacture and/or kits that include a composition comprising a therapeutically effective amount of any of the engineered cells described herein, an agent such as an anti-inflammatory agent or agent that reduces microglial cell activity or function, such as an inhibitor of microglial cell activity, e.g. CSF1R inhibitor, or DMF, and instructions for administering, to a subject for treating a disease or condition. In some embodiments, the instructions can specify some or all of the elements of the methods provided herein. In some embodiments, the instructions specify particular instructions for administration of the cells for cell therapy, e.g., doses, timing, selection and/or identification of subjects for administration and conditions for administration of the T cell therapy and/or the agent such as agent that is anti-inflammatory or anti-oxidative or that reduces microglial cell activity, such as an inhibitor of microglial cell activity, e.g. CSF1R inhibitor or DMF, In some embodiments, the articles of manufacture and/or kits further comprise an agent for lymphodepleting therapy, and optionally further includes instructions for administering the lymphodepleting therapy. In some embodiments, the instructions can be included as a label or package insert accompanying the compositions for administration.

The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include (a) a first container with a composition contained therein, wherein the composition includes the antibody or engineered cells used for the immunotherapy, e.g. T cell therapy; and (b) a second container with a composition contained therein, wherein the composition includes the second agent, such as the agent (such as agent for reducing inflammatory effects such as one or more microglial cell activity, such as an inhibitor of microglial cell activity, e.g. a CSF1R inhibitor or DMF). In some embodiments, the article of manufacture or kit comprises a container, optionally a vial comprising a plurality of CD4+ T cells expressing a recombinant receptor, a container, optionally a vial comprising a plurality of CD8+ T cells expressing a recombinant receptor, and a container containing an agent for reducing microglial cell activity, such as an inhibitor of microglial cell activity, e.g. a CSF1R inhibitor or DMF. In some embodiments, the article of manufacture or kit comprises a container, optionally a vial comprising a plurality of CD4+ T cells expressing a recombinant receptor, and further comprises, in the same container, a plurality of CD8+ T cells expressing a recombinant receptor. In some embodiments, a cryoprotectant is included with the cells. In some aspects the container is a bag.

In some embodiments, the article of manufacture or kit comprises a plurality of CD4+ T cells expressing a recombinant receptor, and instructions for administering, to a subject having a disease or condition, all or a portion of the plurality of CD4+ T cells and further administering CD8+ T cells expressing a recombinant receptor. In some embodiments, the instructions specify administering the CD4+ T cells prior to administering the CD8+ cells. In some cases, the instructions specify administering the CD8+ T cells prior to administering the CD4+ cells. In some embodiments, the article of manufacture or kit comprises a plurality of CD8+ T cells expressing a recombinant receptor, and instructions for administering, to a subject having a disease or condition, all or a portion of the plurality of CD8+ T cells and CD4+ T cells expressing a recombinant receptor. In some embodiments, the instructions specify dosage regimen and timing of the administration of the cells and the agent such as the agent for reducing microglial cell activity.

In some embodiments, the articles of manufacture and/or kits further include one or more agents or treatments for treating, preventing, delaying, reducing or attenuating the development or risk of development of a toxicity and/or instructions for the administration of one or more agents or treatments for treating, preventing, delaying, reducing or attenuating the development or risk of development of a toxicity in the subject. In some embodiments, the agent is an inhibitor of microglial cell activity described herein.

In some embodiments, the instructions are included which specify administering the agent such as the agent for reducing microglial cell activity or inflammation or oxidative stress response functions sequentially, intermittently, or at the same time as or in the same composition as cells for adoptive cell therapy. For example, the instructions are provided that specify that the agent can be administered prior to, during, simultaneously with, or after administration of the cell therapy. In some embodiments, the instructions specify administering the agent prior to administration of the cell therapy. In some embodiments, the instructions specify that the agent is not further administered after initiation of the cell therapy or administering the agent after administration of the cell therapy.

In some embodiments, the articles of manufacture and/or kits further include one or more additional agents for therapy, e.g., lymphodepleting therapy and/or combination therapy, as described herein, and optionally instructions for administering the additional agents. In some examples, the articles of manufacture may further contain one or more therapeutic agents. In some embodiments, the therapeutic agent is an immunomodulatory agent, a cytotoxic agent, an anti-cancer agent or a radiotherapeutic.

The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, a “subject” is a mammal, such as a human or other animal, and typically is human. In some embodiments, the subject, e.g., patient, to whom the immunomodulatory polypeptides, engineered cells, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or engineered cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the immunomodulatory polypeptides or engineered cells administered. In some embodiments, the provided methods involve administering the immunomodulatory polypeptides, engineered cells, or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about λ” includes description of “λ”.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section heading used herein are for organizational purposes only and are not to be construed as limiting the subject matter described.

### VII. Exemplary Embodiments

Among the exemplary embodiments are:

1. A method of treatment, comprising administering to a subject having a disease or condition a therapeutic agent for treating a disease or condition, wherein:

administration of the therapeutic agent is or is suspected of being associated with a risk of eliciting a toxic outcome or symptom; and

the subject has been administered, prior to initiation of the therapy, an agent capable of preventing, blocking or reducing inflammation, oxidative stress response effects, and/or one or more microglial cell activity or function, and/or of promoting an anti-inflammatory or protective phenotype of an immune cell such as an immune cell in the CNS such as a microglial cell.

2. The method of embodiment 1, wherein the prior administration of the agent is in an amount effective to prevent, block or reduce inflammation, oxidative stress response effects, and/or one or more microglial cell activity or function in the subject and/or to promote anti-inflammatory or protective phenotype of an immune cell such as an immune cell in the CNS such as a microglial cell.

3. The method of embodiment 1 or embodiment 2, further comprising, prior to administering the therapy:

administering to the subject the agent capable of preventing, blocking or reducing or altering microglial cell activity or a phenotype thereof;

administering to the subject the agent capable of promoting an anti-inflammatory or protective phenotype of an immune cell such as an immune cell in the CNS such as a microglial cell; and/or

administering to the subject the agent capable of preventing, blocking or reducing inflammation, oxidative stress response effects.

4. A method of treatment, comprising:

(a) administering to a subject an agent capable of preventing, blocking or reducing inflammation, oxidative stress response effects, and/or one or more microglial cell activity or function or phenotype and/or of promoting an anti-inflammatory or protective phenotype of an immune cell such as an immune cell in the CNS such as a microglial cell; and

(b) after the administration in (a), administering to the subject having a disease or condition a therapeutic agent for treating a disease or condition, wherein administration of the therapeutic agent is or is suspected of being associated with a risk of eliciting a toxic outcome or symptom.

5. The method of embodiment 4, wherein the agent is administered in an amount effective to prevent, block or reduce inflammation, oxidative stress response effects, and/or one or more microglial cell activity or function in the subject and/or to promote anti-inflammatory or protective phenotype of an immune cell such as an immune cell in the CNS such as a microglial cell activity or function in the subject.

6. The method of any of embodiments 1-5, wherein the toxic outcome or symptom is associated with neurotoxicity or cytokine release syndrome (CRS).

7. The method of any of embodiments 1-6, wherein:

the toxic outcome or symptom is associated with severe neurotoxicity and/or is associated with grade 2 or higher or grade 3 or higher neurotoxicity; and/or

the toxic outcome or symptom is associated with severe CRS and/or is associated with grade 2 or higher or grade 3 or higher CRS.

8. The method of any of embodiments 1-7, wherein the toxic outcome is cerebral edema or is associated with cerebral edema.

9. The method of any of embodiments 1-8, wherein administration of the agent is started at a time point that is within or within about 1 hour, 2 hours, 6 hours, 12 hours, 24 hours, 3 days, 6 days, 12 days, 15 days, 30 days, 60 days or 90 days or more prior to administration of the therapy.

10. The method of any of embodiments 1-9, wherein the agent is administered greater than 4 days prior to initiation of the therapy.

11. The method of any of embodiments 1-10, wherein:

the therapy is not or does not comprise interleukin 2 (IL-2);

the subject has not previously received administration of IL-2 prior to administration of the therapy; or

the subject has not received administration of IL-2 greater than 4 days prior to initiation of the therapy.

12. The method of any of embodiments 1-11, wherein the agent is not further administered after administration of the therapeutic agent.

13. The method of any of embodiments 1-12, wherein the method further comprises administering the agent concurrently with or after administration of the therapeutic agent.

14. The method of embodiment 12, wherein the agent is administered within or within about 1 day, 2 days, 3 days, four days, five days, six days or seven days after administration of the therapeutic agent.

15. A method of treatment, comprising:

(a) administering to a subject having a disease or condition a therapeutic agent for treating a disease or condition, wherein administration of the therapeutic agent is or is suspected of being associated with a risk of eliciting a toxic outcome or symptom of or related to severe CRS or severe neurotoxicity in the subject and/or grade 2 or grade 3 or higher CRS or grade 2 or grade 3 or higher neurotoxicity in the subject; and

(b) administering to the subject an agent capable of preventing, blocking or reducing inflammation, oxidative stress response effects, and/or one or more microglial cell activity or function and/or of promoting an anti-inflammatory or protective phenotype of an immune cell such as an immune cell in the CNS such as a microglial cell, wherein the agent is administered (i) at a time that is within or within about 1 day, 2 days, 3 days, four days, five days, six days or seven days after administration of the therapeutic agent and/or (ii) at or about or within 24 hours of the subject exhibiting a first sign or symptom indicative of CRS or neurotoxicity after administration of the therapy.

16. The method of embodiment 15, wherein the agent is administered in an amount effective to prevent, block or reduce inflammation, oxidative stress response effects, and/or one or more microglial cell activity or function in the subject and/or to promote anti-inflammatory or protective phenotype of an immune cell such as an immune cell in the CNS such as a microglial cell activity or function in the subject.

17. The method of embodiment 15 or embodiment 16, wherein the first sign or symptom indicative of CRS or neurotoxicity is a fever.

18. A method of treatment, comprising:

(a) administering to a subject having a disease or condition a therapeutic agent for treating a disease or condition, wherein the therapeutic agent is or is suspected of being associated with a risk of eliciting a toxic outcome or symptom; and

(b) administering to the subject an agent capable of preventing, blocking or reducing inflammation, oxidative stress response effects, and/or one or more microglial cell activity or function or phenotype and/or of promoting an anti-inflammatory or protective phenotype of an immune cell such as an immune cell in the CNS such as a microglial cell, wherein the agent is administered at or about or within 24 hours of the subject exhibiting a fever after administration of the therapeutic agent.

19. The method of embodiment 18, wherein the agent is administered in an amount effective to prevent, block or reduce prevent, block or reduce inflammation, oxidative stress response effects, and/or one or more microglial cell activity or function in the subject and/or to promote anti-inflammatory or protective phenotype of an immune cell such as an immune cell in the CNS such as a microglial cell activity or function in the subject.

20. The method of any of embodiments 17-19, wherein the fever comprises a temperature of at least or at least about 38.0° C.

21. The method of any of embodiments 17-20, wherein:

the fever comprises a temperature that is between or between about 38.0° C. and 42.0° C., 38.0° C. and 39.0° C., 39.0° C. and 40.0° C. or 40.0° C. and 42.0° C., each inclusive; or

the fever comprises a temperature that is greater than or greater than about or is or is about 38.5° C., 39.0°, 39.5° C., 40.0° C., 41.0° C., 42.0° C.

22. The method of any of embodiments 17-21, wherein the fever is a sustained fever.

23. The method of embodiments 17-22, wherein the fever is a fever that is not reduced or not reduced by more than 1° C. after treatment with an antipyretic and/or wherein the fever has not been reduced by more than 1° C., following treatment of the subject with an antipyretic.

24. The method of embodiment 15 or embodiment 16, wherein the first sign or symptom indicative of CRS or neurotoxicity is an altered level of one or more biomarkers in a sample from the subject compared to in the sample prior to administration of the therapeutic agent.

25. The method of embodiment 24, wherein the sample is a serum or blood sample.

26. The method of embodiment 24 or embodiment 25, wherein the sample is obtained or has been obtained from the subject no more than 3 days, no more than 2 days or no more than 1 day after initiation of the therapy or a first administration of the therapeutic agent.

27. The method of any of embodiments 24-26, wherein the altered level is an increased level of the one or more biomarker, optionally increased greater than or greater than about 1.5-fold, 2-fold, 3-fold, 4-fold, 5-fold, 10-fold, 20-fold or 50-fold.

28. The method of any of embodiments 24-27, further comprising assessing the sample from the subject for the one or more biomarkers after administration of the therapeutic agent and prior to administration of the agent.

29. The method of any of embodiments 13-28, wherein administration of the agent is continued after initiation of administration of the therapeutic agent until the risk or suspected risk of a toxic outcome or symptom in the subject from administration of the therapeutic agent has subsided or is not present.

30. A method of ameliorating toxicity induced by or associated with administration of a therapeutic agent, the method comprising:

(a) administering to a subject having a disease or condition a therapeutic agent for treating a disease or condition, wherein the therapeutic agent is or is suspected of being associated with a risk of eliciting a toxic outcome or symptom; and

(b) administering to the subject an agent capable of preventing, blocking or reducing inflammation, oxidative stress response effects, and/or one or more microglial cell activity or function or phenotype and/or of promoting an anti-inflammatory or protective phenotype of an immune cell such as an immune cell in the CNS such as a microglial cell activation or function, wherein the agent is administered in a dosage regimen until the risk or suspected risk of a toxic outcome or symptom associated with administration of the therapeutic agent has subsided or is not present.

31. The method of embodiment 30, wherein the agent is administered in an amount effective to prevent, block or reduce inflammation, oxidative stress response effects, and/or one or more microglial cell activity or function in the subject and/or to promote anti-inflammatory or protective phenotype of an immune cell such as an immune cell in the CNS such as a microglial cell activity or function in the subject.

32. The method of embodiment 30 or embodiment 31, wherein the agent is administered prior to, simultaneously with and/or subsequent to initiation of administration of the therapeutic agent.

33. The method of any of embodiments 13-32, wherein the inhibitor is administered for a time period up to 2 days, up to 7 days, up to 14 days, up to 21 days, up to 28 days, up to 35 days, up to 42 days, up to two months, up to three months, up to 6 months or up to 1 year after initiation of the administration of the therapeutic agent.

34. The method of any of embodiments 29-33, wherein the agent is administered for a time period until:

the grade of CRS or neurotoxicity in the subject is reduced to a lower grade compared to prior to administration of the agent or compared to a preceding time point after administration of the agent; or a sign or symptom of grade 1 or higher or grade 2 or higher CRS or neurotoxicity is not present or detectable in the subject after administration of the agent.

35. The method of any of embodiments 1-34, wherein, prior to the administration, the subject has been preconditioned with a lymphodepleting therapy comprising one or more chemotherapeutic agent.

36. The method of any of embodiments 1-35, further comprising, prior to the administration of the therapeutic agent, administering to the subject a lymphodepleting therapy comprising one or more chemotherapeutic agent.

37. The method of embodiment 35 or embodiment 36, wherein the chemotherapeutic agent comprises an agent selected from the group consisting of cyclophosphamide, fludarabine, and/or a combination thereof.

38. The method of embodiment 37, wherein:

the chemotherapeutic agent is or comprises fludarabine that is administered at a dose of between or between about 1 mg/m² and 100 mg/m², between or between about 10 mg/m² and 75 mg/m², between or between about 15 mg/m² and 50 mg/m², between or between about 20 mg/m² and 30 mg/m², or between or between about 24 mg/m² and 26 mg/m²; and/or

the chemotherapeutic agent is cyclophosphamide that is administered between or between about 20 mg/kg and 100 mg/kg, between or between about 40 mg/kg and 80 mg/kg or between or between about 30 mg/kg and 60 mg/kg.

39. The method of embodiment 37 or embodiment 38, wherein the cyclophosphamide is administered once daily for one or two days, and/or the fludarabine is administered daily for 3-5 days.

40. The method of any of embodiments 37-39, wherein the lymphodepleting therapy comprises administration of cyclophosphamide between or between about 30 mg/kg and 60 mg/kg and administration of fludarabine between or between about 25 mg/m² and 30 mg/m² for three days.

41. The method of any of embodiments 35-40, wherein the lymphodepleting therapy is initiated at a time that is at least at or about 2 days prior to or is between at or about 2 days and at or about 7 days prior to the administration of the therapeutic agent.

42. The method of any of embodiments 1-41, wherein the therapeutic agent is an immunotherapy.

43. The method of any of embodiments 1-42, wherein the therapeutic agent is a T cell therapy or is a T cell-engaging therapy.

44. The method of embodiment 43, wherein the therapeutic agent is a T cell-engaging therapy comprising a bispecific antibody, wherein at least one binding portion specifically binds to a T cell antigen, optionally CD3.

45. The method of embodiment 43, wherein the cell therapy is an adoptive cell therapy.

46. The method of any of embodiments 1-43 and 45, wherein the therapeutic agent is a T cell therapy that is or comprises tumor infiltrating lymphocytic (TIL) therapy or a T cell therapy comprising genetically engineered cells expressing a recombinant receptor that specifically binds to a ligand.

47. The method of embodiment 45, wherein the T cell therapy is or comprises genetically engineered cells expressing a recombinant receptor that specifically binds to a ligand.

48. The method of any of embodiments 1-47, wherein the agent reduces the expression of a microglial activation marker on microglial cells, reduces the level or amount one or more effector molecule associated with microglial cell activation in a biological sample; alters microglial cell homeostasis; decreases or blocks microglial cell proliferation; and/or reduces or eliminates microglial cells.

49. The method of any of embodiments 1-48, wherein the agent reduces or eliminates microglial cells and the reduction in the number of microglial cells is by greater than 20%, greater than 30%, greater than 40% or greater than 50%, greater than 60%, greater than 70%, greater than 80%, greater than 90%, greater than 95% or greater than 99% compared to the number of microglial cells at a time just prior to initiation of the administration of the agent.

50. The method of any of embodiments 1-48, wherein:

the agent reduces the expression of a microglial activation marker, optionally CD86 and CD68; and/or

the agent reduces the level or amount of one or more effector molecule, wherein the one or more effector molecule is a optionally or one or more pro-inflammatory mediator, optionally selected from one or more of inducible nitric oxide synthase (iNOS), prostaglandin E(2) (PGE(2)), IL-6, IL-1β, IL-8, CCL2, CXCL10, TNF-α, CCL7, CXCL5, CXCL9, CXCL6, MMP-7, MMP-2, and MMP-9.

51. The method of any of embodiments 48-50, wherein the biological sample is a brain, serum or plasma sample.

52. The method of any of embodiments 1-51, wherein the agent is selected from an anti-inflammatory agent, an inhibitor of NADPH oxidase (NOX2), a calcium channel blocker, a sodium channel blocker, inhibits GM-CSF, inhibits CSF1R, specifically binds CSF-1, specifically binds IL-34, inhibits the activation of nuclear factor kappa B (NF-κB), activates a CB₂ receptor and/or is a CB₂ agonist, a phosphodiesterase inhibitor, inhibits microRNA-155 (miR-155), upregulates microRNA-124 (miR-124), inhibits nitric oxide production, inhibits nitric oxide synthase, or activates NRF2.

53. The method of any of embodiments 1-52, wherein the prevention, block or reduction of microglial cell activation or function by the agent is transient and/or is reversible upon discontinued administration of the agent.

54. The method of any of embodiments 1-53, wherein the agent is or comprises a small molecule, peptide, protein, antibody or antigen-binding fragment thereof, an antibody mimetic, an aptamer, or a nucleic acid molecule.

55. The method of embodiment 54, wherein the agent is selected from minocycline, naloxone, nimodipine, Riluzole, MOR103, lenalidomide, a cannabinoid (optionally WIN55 or 212-2), intravenous immunoglobulin (IVIg), ibudilast, anti-miR-155 locked nucleic acid (LNA), MCS110, PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945, emactuzumab, IMC-CS4, FPA008, LY-3022855, AMG-820, TG-3003, dimethyl fumarate, and natalizumab.

56. The method of any of embodiments 1-55, wherein the agent is an inhibitor of colony stimulating factor 1 receptor (CSF1R).

57. The method of embodiment 56, wherein the inhibitor transiently inhibits the activity of the CSF1R and/or wherein the inhibition of CSF1R activity is not permanent.

58. The method of any of embodiments 1-57, wherein:

the inhibitor is selected from PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof;

emactuzumab, IMC-CS4, FPA008, LY-3022855, AMG-820 and TG-3003;

or a combination of any of the foregoing.

59. The method of any of embodiments 1-58, wherein the inhibitor is PLX-3397.

60. The method of any of embodiments 1-59, wherein the agent is an inhibitor of nitric oxide synthase.

61. The method of embodiment 60, wherein the inhibitor of nitric oxide synthase is selected from VAS-203, cindunistat, A-84643, ONO-1714, L-NOARG, NCX-456, VAS-2381, GW-273629, NXN-462, CKD-712, KD-7040, and guanidinoethyldisulfide.

62. The method of any of embodiments 1-61, wherein the agent is an activator of NRF2.

63. The method of embodiment 62, wherein the activator of NRF2 is dimethyl fumarate.

64. The method of any of embodiments 1-63, wherein the agent sequesters T cells from the central nervous system.

The method of embodiment 64, wherein the agent modulates a sphingosine-1-phosphate (S1P) receptor.

66. The method of embodiment 65, wherein the S1P receptor is a S1PR1 and/or a S1PR5.

67. The method of any of embodiments 64-66, wherein the agent is fingolimod (Gilenya®) or ozanimod (RPC-1063).

68. A method of treatment, comprising administering, to a subject having a disease or condition, a cell therapy for treating a disease or condition, wherein the cell therapy comprises cells that secrete an inhibitor of colony-stimulating factor-1 receptor (CSF1R).

69. The method of embodiment 68, wherein the cell therapy is a T cell therapy. 70. The method of embodiment 68 or embodiment 69, wherein the inhibitor is a peptide, polypeptide or antibody or antigen-binding fragment thereof.

71. The method of any of embodiments 68-70, wherein the inhibitor is an antibody or antigen-binding fragment thereof.

72. The method of any of embodiments 68-71, wherein the inhibitor is selected from emactuzumab, IMC-CS4, FPA008, LY-3022855, AMG-820, TG-3003 or is an antigen-binding fragment thereof.

73. The method of any of embodiments 1-14 and 30-72, wherein the therapeutic agent is administered after administering the agent at a time at which microglial cell activation or function is reduced, blocked or prevented or is likely to be reduced, blocked or prevented in the subject or at a time in which a parameter associated with activity of the agent is altered in the subject.

74. The method of any of embodiments 1-14 and 30-73, wherein the therapeutic agent is administered after administering the agent at a time at which:

(i) the number of microglial cells is reduced or eliminated in the subject compared to just prior to initiation of administration of the agent; or

(ii) there exists a reduction in the level or amount of a proinflammatory mediator of microglial cell activation in a sample, optionally a brain, serum or plasma sample, from the subject compared to just prior to initiation of administration of the agent;

(iii) the expression of a microglial cell activation marker, optionally CD86 or CD68, is reduced compared to just prior to initiation of administration of the agent;

(iv) there is an increase in the plasma or serum level of CSF-1 or IL-34 compared to just prior to initiation of administration of the agent;

(v) there is a reduction of Kupffer cells and/or an increase in the level or amount of a serum enzyme associated with reduction of Kupffer cells compared to just prior to initiation of administration of the agent;

(vi) there is a reduction in the number of tumor-associated macrophages (TAM) compared to just prior to initiation of administration of the agent; and/or

(vi) there is a decrease in CD14dim/CD16+ nonclassical monocytes in peripheral blood compared to just prior to initiation of administration of the agent.

75. The method of any of embodiments 1-14 and 30-74, further comprising after administering the agent but prior to administering the therapeutic agent assessing a sample from the subject for a prevention, block or reduction in microglial cell activation or function or for alteration of a parameter associated with activity of the agent.

76. The method of any of embodiments 1-14 and 30-75, further comprising after administering the agent but prior to administering the therapeutic agent assessing a sample from the subject for one or more of:

(i) a reduction or elimination of microglial cells in the subject compared to just prior to initiation of administration of the agent; or

(ii) a reduction in the level or amount of a proinflammatory mediator of microglial cell activation in a sample, optionally a brain, serum or plasma sample, from the subject compared to just prior to initiation of administration of the agent;

(iii) a reduction in expression of a microglial cell activation marker, optionally CD86 or CD68, compared to just prior to initiation of administration of the agent;

(iv) an increase in the plasma or serum level of CSF-1 or IL-34 compared to just prior to initiation of administration of the agent;

(v) a reduction of Kupffer cells and/or an increase in the level or amount of a serum enzyme associated with reduction of Kupffer cells compared to just prior to initiation of administration of the agent;

(vi) a reduction in the number of tumor-associated macrophages (TAM) compared to just prior to initiation of administration of the agent; and/or

(vi) a decrease in CD14dim/CD16+ nonclassical monocytes in peripheral blood compared to just prior to initiation of administration of the agent.

77. The method of embodiment 74 or embodiment 76, wherein the serum enzyme is selected from alanine aminotransferase (ALT), AST, creatine kinase (CK) and LDH.

78. The method of embodiment 74 or embodiment 76, wherein the serum cytokine is selected from nitric oxide synthase (iNOS), prostaglandin E(2) (PGE(2)), IL-6, IL-1β, IL-8, CCL2, CXCL10, TNF-α, CCL7, CXCL5, CXCL9, CXCL6, MMP-7, MMP-2, and MMP-9.

79. The method of any of embodiments 74 and 76-78, wherein the reduction or increase is by greater than or greater than about 1.2-fold, 1.5-fold, 2-fold, 3-fold, 4-fold, 5-fold, 10-fold or more.

80. The method of any of embodiments 1-79, wherein the toxic outcome or symptom in the subject is reduced or ameliorated compared to a method in which the therapeutic agent is administered to the subject in the absence of the agent.

81. The method of embodiment 80, wherein the toxic outcome or symptom is associated with neurotoxicity or cytokine release syndrome (CRS), which optionally is severe neurotoxicity or severe CRS.

82. The method of embodiment 80 or embodiment 81, wherein the toxic outcome or symptom in the subject at up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent is not detectable or is reduced as compared to a method in which the therapeutic agent is administered to the subject in the absence of the agent.

83. The method of any of embodiments 80-82, wherein the toxic outcome or symptom is reduced by greater than or greater than about 1.2-fold, 1.5-fold, 2-fold, 3-fold, 4-fold, 5-fold, 10-fold or more.

84. The method of any of embodiments 1-83, wherein the toxic outcome or symptom is associated with neurotoxicity.

85. The method of embodiment 84, wherein the neurotoxicity is severe neurotoxicity and/or the neurotoxicity is a grade 3 or higher neurotoxicity.

86. The method of embodiment 84 or embodiment 85, wherein the toxic outcome or symptom is associated with grade 3, grade 4 or grade 5 neurotoxicity.

87. The method of any of embodiments 1-86, wherein the toxic outcome or symptoms is one or more of confusion, delirium, expressive aphasia, obtundation, myoclonus, lethargy, altered mental status, convulsions, seizure-like activity, seizures (optionally as confirmed by electroencephalogram [EEG]), cerebral edema, elevated levels of beta amyloid (Aβ), elevated levels of glutamate, and elevated levels of oxygen radicals, encephalopathy, dysphasia, tremor, choreoathetosis, symptoms that limit self-care, symptoms of peripheral motor neuropathy, symptoms of peripheral sensory neuropathy and combinations thereof.

88. The method of any of embodiments 84-87, wherein a toxic outcome or symptom of neurotoxicity in the subject at day up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent is not detectable or is reduced as compared to a method in which the therapeutic agent is administered to the subject in the absence of the agent.

89. The method of embodiment 88, wherein the toxic outcome or symptom of neurotoxicity is reduced by greater than or greater than about 1.2-fold, 1.5-fold, 2-fold, 3-fold, 4-fold, 5-fold, 10-fold or more.

90. The method of any of embodiments 84-89, wherein the method is such that:

(i) the administration of the therapeutic agent does not induce neurotoxicity in the subject or does not induce severe neurotoxicity in the subject;

(ii) the administration of the therapeutic agent does not induce grade 3 or higher neurotoxicity in the subject, does not induce grade 2 or higher neurotoxicity in the subject or does not induce grade 1 or higher neurotoxicity in the subject;

(iii) based on clinical data, administration of the therapeutic agent does not induce neurotoxcity or does not induce severe neurotoxicity in a majority of subjects so treated; or

(iv) based on clinical data, administration of the therapeutic agent does not result in a toxic outcome or symptom of neurotoxicity greater than grade 3, greater than grade 2 or greater than grade 1 in a majority of the subjects to treated.

91. The method of any of embodiments 1-90, wherein the toxic outcome or symptom is cerebral edema or is associated with cerebral edema.

92. The method of embodiment 9l, wherein the method is such that:

(i) the administration of the therapeutic agent does not induce cerebral edema in the subject; or

(ii) based on clinical data, a majority of subjects so treated do not exhibit a cerebral edema after the administration of the therapeutic agent.

93. The method of any of embodiments 1-92, wherein the toxic outcome or symptom is associated with cytokine-release syndrome (CRS).

94. The method of embodiment 93, wherein the CRS is severe CRS and/or the CRS is grade 3 or higher CRS.

95. The method of embodiment 93 or embodiment 94, wherein the toxic outcome or symptom is associated with grade 3, grade 4 or grade 5 CRS.

96. The method of any of embodiments 93-95, wherein the toxic outcome or symptom is one or more of persistent fever, hypotension, hypoxia, neurologic disturbances, or elevated serum level of an inflammatory cytokine or C reactive protein (CRP).

97. The method of any of embodiments 93-96, wherein toxic outcome or symptom of CRS in the subject at day up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent is not detectable or is reduced as compared to a method in which the therapeutic agent is administered to the subject in the absence of the agent.

98. The method of embodiment 97, wherein the CRS is reduced by greater than or greater than about 1.2-fold, 1.5-fold, 2-fold, 3-fold, 4-fold, 5-fold, 10-fold or more.

99. The method of any of embodiments 93-98, wherein the method is such that:

(i) the administration of the therapeutic agent does not induce CRS in the subject or does not induce severe CRS in the subject;

(ii) the administration of the therapeutic agent does not induce grade 3 or higher CRS in the subject, does not induce grade 2 or higher CRS in the subject or does not induce grade 1 or higher CRS in the subject;

(iii) based on clinical data, administration of the therapeutic agent does not induce CRS or does not induce severe CRS in a majority of subjects so treated;

(iv) based on clinical data, administration of the therapeutic agent does not result in a toxic outcome or symptom of CRS greater than grade 3, greater than grade 2 or greater than grade 1 in a majority of the subjects to treated.

100. The method of any of embodiments 1-99, wherein the disease or condition is a tumor or a cancer.

101. The method of any of embodiments 1-100, wherein the disease or condition is a leukemia or lymphoma.

102. The method of any of embodiments 1-101, wherein the disease or condition is a non-Hodgkin lymphoma (NHL), an acute lymphoblastic leukemia (ALL) or a chronic lymphocytic leukemia (CLL).

103. The method of any of embodiments 47-102, wherein the recombinant receptor binds to, recognizes or targets an antigen associated with a disease or condition.

104. The method of any of embodiments 47-103, wherein the recombinant receptor is a T cell receptor or a functional non-T cell receptor.

105. The method of any of embodiments 47-104, wherein the recombinant receptor is a chimeric antigen receptor (CAR).

106. The method of embodiment 105, wherein the CAR comprises an extracellular antigen-recognition domain that specifically binds to the antigen and an intracellular signaling domain comprising an ITAM.

107. The method of any of embodiments 103-106, wherein the antigen is CD19.

108. The method of embodiment 106 or embodiment 107, wherein the intracellular signaling domain comprises an intracellular domain of a CD3-zeta (CD3ζ) chain.

109. The method of any of embodiments 105-108, wherein the CAR further comprises a costimulatory signaling region.

110. The method of embodiment 109, wherein the costimulatory signaling domain comprises a signaling domain of CD28 or 4-1BB.

111. The method of any of embodiments 45-110, wherein the cells of the cell therapy are CD4+ or CD8+ T cells.

112. The method of any of embodiments 45-111, wherein the cells of the cell therapy are autologous to the subject.

113. The method of any of embodiments 45-112, wherein the cells are allogeneic to the subject.

114. The method of any of embodiments 45-113, wherein the therapeutic agent is administered in a sufficient dose, without the administration of the agent, to reduce burden of the disease or condition in the subject as indicated by one or more factors indicative of disease burden, wherein the disease burden optionally is a tumor burden.

115. The method of embodiment 114, wherein the reduction in burden comprises a reduction in total number of cells of the disease in the subject, in an organ of the subject, in a tissue of the subject, or in a bodily fluid of the subject, a reduction in mass or volume of a tumor, and/or a reduction in number and/or extent of metastases.

116. The method of embodiment 114 or embodiment 115, wherein:

the dose of cells is sufficient, without administration of the agent, to result in partial remission or complete remission in a majority of subjects so treated with the dose of cells; or

the disease or condition is a cancer and the dose of cells is sufficient, without administration of the agent, to reduce burden of disease from morphological disease to detectable molecular disease and/or minimum residual disease in a majority of subjects so treated; and/or

the disease is a leukemia or lymphoma and the dose of cells is sufficient, without administration of the agent, to reduce the blast cells in the bone marrow to less than or about less than 5%.

117. The method of any of embodiments 45-116, wherein the cell therapy is administered in a sufficient dose, without the administration of the agent, such that:

there is a maximum concentration or number of cells of the cell therapy in the blood of the subject of at least at or about 10 cells of the cell therapy per microliter, at least 50% of the total number of peripheral blood mononuclear cells (PBMCs), at least at least about 1×10⁵ cells of the cell therapy, or at least 5,000 copies of recombinant receptor-encoding DNA per micrograms DNA; and/or

at day 90 following the initiation of the administration, cells of the cell therapy are detectable in the blood or serum of the subject; and/or

at day 90 following the initiation of the administration, the blood of the subject contains at least 20% cells of the cell therapy, at least 10 cells of the cell therapy per microliter or at least 1×10⁴ recombinant receptor-expressing cells.

118. The method of any of embodiments 45-117, wherein the cell therapy comprises administration of a dose comprising a number of cells between or between about 0.5×10⁶ cells/kg body weight of the subject and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells·kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

119. The method of any of embodiments 45-118, wherein:

the dose of cells is a dose that, when administered in the absence of the agent, does, or is likely to, result in severe CRS or grade 3 or higher CRS in the majority of subjects so treated; or

the dose of cells is a dose that, when administered in the absence of the agent, does, or is likely to, result in severe neurotoxicity or grade 3 or higher neurotoxicity in the majority of subjects so treated.

120. The method of any of embodiments 45-119, wherein the cell therapy is administered at a dose that is higher than a method in which the cell therapy is administered without administering the agent, whereby the agent ameliorates the risk of a toxic outcome to the cell therapy that would occur, or would likely occur, if a similar dose of the cell therapy is administered in the absence of the agent.

121. The method of embodiment 120, wherein the dose is at least 1.5-fold, 2-fold, 3-fold, 4-fold, 5-fold or 10-fold greater.

122. The method of any of embodiments 45-121, wherein the cell therapy comprises administration of a dose comprising a number of cells

between about 2×10⁶ cells per kilogram (cells/kg) body weight and about 6×10⁶ cells/kg, between about 2.5×10⁶ cells/kg and about 5.0×10⁶ cells/kg, or between about 3.0×10⁶ cells/kg and about 4.0×10⁶ cells/kg, each inclusive;

between about 1.5×10⁸ cells and 4.5×10⁸ cells, between about 1.5×10⁸ cells and 3.5×10⁸ cells or between about 2×10⁸ cells and 3×10⁸ cells, each inclusive; or

between about 1.5×10⁸ cells/m² and 4.5×10⁸ cells/m², between about 1.5×10⁸ cells/m² and 3.5×10⁸ cells/m² or between about 2×10⁸ cells/m² and 3×10⁸ cells/m², each inclusive.

123. The method of any of embodiments 45-122, wherein the cell therapy is administered as a single pharmaceutical composition comprising the cells.

124. The method of any of embodiments 45-123, wherein the cell therapy comprises a dose of cell that is a split dose, wherein the cells of the dose are administered in a plurality of compositions, collectively comprising the cells of the dose, over a period of no more than three days.

125. The method of any of embodiments 1-124, wherein:

the agent is administered, or each administration of the agent is independently administered, in a dosage amount of from or from about 0.2 mg per kg body weight of the subject (mg/kg) to 200 mg/kg, 0.2 mg/kg to 100 mg/kg, 0.2 mg/kg to 50 mg/kg, 0.2 mg/kg to 10 mg/kg, 0.2 mg/kg to 1.0 mg/kg, 1.0 mg/kg to 200 mg/kg, 1.0 mg/kg to 100 mg/kg, 1.0 mg/kg to 50 mg/kg, 1.0 mg/kg to 10 mg/kg, 10 mg/kg to 200 mg/kg, 10 mg/kg to 100 mg/kg, 10 mg/kg to 50 mg/kg, 50 mg/kg to 200 mg/kg, 50 mg/kg to 100 mg/kg or 100 mg/kg to 200 mg/kg; or

the agent is administered, or each administration of the agent is independently administered, in a dosage amount of from or from about 25 mg to 2000 mg, 25 mg to 1000 mg, 25 mg to 500 mg, 25 mg to 200 mg, 25 mg to 100 mg, 25 mg to 50 mg, 50 mg to 2000 mg, 50 mg to 1000 mg, 50 mg to 500 mg, 50 mg to 200 mg, 50 mg to 100 mg, 100 mg to 2000 mg, 100 mg to 1000 mg, 100 mg to 500 mg, 100 mg to 200 mg, 200 mg to 2000 mg, 200 mg to 1000 mg, 200 mg to 500 mg, 500 mg to 2000 mg, 500 mg to 1000 mg or 1000 mg to 2000 mg, each inclusive.

126. The method of any of embodiments 1-125, wherein:

the agent is administered, or each administration of the agent is independently administered, in a dosage amount of at least or at least about or about 0.2 mg per kg body weight of the subject (mg/kg), 1 mg/kg, 3 mg/kg, 6 mg/kg, 10 mg/kg, 20 mg/kg, 30 mg/kg, 50 mg/kg, 100 mg/kg or 200 mg/kg; or

the agent is administered, or each administration of the inhibitor is independently administered, in a dosage amount of at least or at least about 25 mg, 50 mg, 100 mg, 200 mg, 400 mg, 500 mg, 600 mg, 800 mg, 1000 mg, 1200 mg, 1600 mg or 2000 mg.

127. The method of any of embodiments 1-126, wherein the agent is administered daily, every other day, once a week or once a month.

128. The method of any of embodiments 1-127, wherein the agent is administered daily in a dosage amount of at least or at least about 25 mg/day, 50 mg/day, 100 mg/day, 200 mg/day, 400 mg/day, 500 mg/day, 600 mg/day, 800 mg/day, 1000 mg/day, 1200 mg/day, 1600 mg/day or 2000 mg/day.

129. The method of any of embodiments 1-128, wherein the inhibitor is administered orally, subcutaneous or intravenously.

130. The method of any of embodiments 1-129, wherein the subject is a human subject.

131. A combination, comprising:

a first composition comprising genetically engineered cells expressing a recombinant receptor that specifically binds to an antigen; and

a second composition comprising an inhibitor of colony stimulating factor 1 receptor (CSF1R) or a modulator of NRF2 or an NRF2-related pathway.

132. The combination of embodiment 131, wherein the inhibitor reduces the expression of a microglial activation marker on microglial cells, reduces the level or amount one or more effector molecule associated with microglial cell activation in a biological sample; alters microglial cell homeostasis; decreases or blocks microglial cell proliferation; and/or reduces or eliminates microglial cells.

133. The combination of embodiment 132, wherein the inhibition of CSF-1R and/or the reduction of microglial cell activation by the agent is transient and/or is reversible upon discontinued administration of the agent.

134. The combination of any of embodiments 131-133, wherein the inhibitor is a small molecule, peptide, protein, antibody or antigen-binding fragment thereof, an antibody mimetic, an aptamer, or a nucleic acid molecule.

135. The combination of any of embodiments 131-134, wherein the inhibitor is selected from: PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof;

emactuzumab, IMC-CS4, FPA008, LY-3022855, AMG-820 and TG-3003;

or a combination of any of the foregoing.

136. The combination of any of embodiments 131-135, wherein the inhibitor is PLX-3397.

137. The combination of any of embodiments 131-136, wherein the recombinant receptor binds to, recognizes or targets an antigen associated with a disease or condition.

138. The combination of any of embodiments 131-137, wherein the recombinant receptor is a T cell receptor or a functional non-T cell receptor.

139. The combination of any of embodiments 131-138, wherein the recombinant receptor is a chimeric antigen receptor (CAR).

140. The combination of embodiment 139, wherein the CAR comprises an extracellular antigen-recognition domain that specifically binds to the antigen and an intracellular signaling domain comprising an ITAM.

141. The combination of any of embodiments 131-140, wherein the antigen is CD19.

142 The combination of embodiment 140 or embodiment 141, wherein the intracellular signaling domain comprises an intracellular domain of a CD3-zeta (CD3ζ) chain.

143. The combination of any of embodiments 139-142, wherein the CAR further comprises a costimulatory signaling region.

144. The combination of embodiment 143, wherein the costimulatory signaling domain comprises a signaling domain of CD28 or 4-1BB.

145. The combination of any of embodiments 131-144, wherein the genetically engineered cells comprise T cells or NK cells.

146. The combination of any of embodiments 131-145, wherein the cells comprise T cells that are CD4+ or CD8+ T cells.

147. The combination of any of embodiments 131-146, wherein the cells are primary cells obtained from a subject, optionally a human subject.

148. The combination of any of embodiments 131-147, wherein the cells are formulated for single dosage administration or multiple dosage administration, which optionally comprises a split dose of cells.

149. The combination of any of embodiments 131-148, wherein the inhibitor is formulated for single dosage administration or multiple dose administration.

150. A kit comprising the combination of any of embodiments 131-149 and, optionally, instructions for administering the compositions to a subject for treating a disease or condition.

151. An article of manufacture, comprising:

(a) a pharmaceutical composition comprising engineered immune cells and/or a T cell-engaging therapy; and

(b) instructions for administration of the composition to a subject having a disease or condition, in combination with an agent capable of reducing or preventing or blocking inflammation, oxidative stress response effects, and/or one or more microglial cell activity or function or phenotype and/or of promoting an anti-inflammatory or protective phenotype of an immune cell such as an immune cell in the CNS such as a microglial cell activation or function in the subject.

152. An article of manufacture, comprising:
(a) a pharmaceutical composition comprising an agent capable of reducing or preventing or blocking inflammation, oxidative stress response effects, and/or one or more microglial cell activity or function or phenotype and/or of promoting an anti-inflammatory or protective phenotype of an immune cell such as an immune cell in the CNS such as a microglial cell activation or function; and
(b) instructions for administration of the composition to a subject having a disease or condition, in combination with an agent for treating said disease or condition, which agent comprises an engineered immune cell and/or T cell-engaging therapy.

153. The kit or article of manufacture of any of embodiments 151-152, wherein the disease or condition is a tumor, optionally a cancer.

154. The kit or article of manufacture of any of embodiments 151-153, wherein the instructions specify the additional therapeutic agent or therapy is for administration prior to, with or at the same time and/or subsequent to initiation of administration of the engineered immune cell and/or T cell-engaging therapy.

155. The kit or article of manufacture of any of embodiments 151-154, wherein the instructions further specify the engineered immune cell and/or T cell-engaging therapy is for parenteral administration, optionally intravenous administration.

156. The kit or article of manufacture of any of embodiments 151-155, wherein the engineered immune cell and/or T cell-engaging therapy comprises primary T cells obtained from a subject.

157. The kit or article of manufacture of embodiment 156, wherein the T cells are autologous to the subject.

158. The kit or article of manufacture of embodiment 156, wherein the T cells are allogeneic to the subject.

159. The kit or article of manufacture of any of embodiments 151-158, wherein the kit or article of manufacture comprises one of a plurality of compositions of the cell therapy comprising a first composition of genetically engineered cells comprising CD4+ T cells or CD8+ T cells, wherein the instructions specify the first composition is for use in with a second composition comprising the other of the CD4+ T cells or the CD8+ T cells, optionally wherein the cells of the first composition and cells of the same composition are from the same subject.

160. The kit or article of manufacture of any of embodiments 151-159, wherein the agent is a small molecule, peptide, protein, antibody or antigen-binding fragment thereof, an antibody mimetic, an aptamer, or a nucleic acid molecule.

161. The kit or article of manufacture of any of embodiments 151-160, wherein the agent is selected from: PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof;

emactuzumab, IMC-CS4, FPA008, LY-3022855, AMG-820 and TG-3003;

or a combination of any of the foregoing.

162. The kit or article of manufacture of any of embodiments 151-161, wherein the agent is PLX-3397.

### VIII. Examples

The following examples are included for illustrative purposes only and are not intended to limit the scope of the invention.

### Example 1: Treatment of Subjects with Combination Therapy of a Microglia Inhibitor, a Cell Therapy, and a Lymphodepleting Therapy

Animal subjects of the non-human primate (NHP), Macaca mulatta, which in aspects closely recapitulates the human immune system are administered cell therapy (T cells expressing a CAR targeting CD20), following administration of an agent to ameliorate toxicity. Thirty days prior to the initiation of cell therapy, T cells are isolated from peripheral blood of Rhesus macaques (n=3) and engineered via transduction with a viral vector encoding a CD20 chimeric antigen receptor (CAR), which is a tumor-associated or tumor-specific antigen, and optionally a detectable marker, such as GFP. Beginning at thirty days prior to the initiation of the cell therapy, subjects in an experimental group receive PLX3397 at 40 mg/kg twice daily. A group of subjects administered vehicle alone is used as a control.

Prior to initiation of cell therapy, blood is obtained from the subjects, lymph node biopsies, and bone marrow aspirates are performed to assess CD20 CAR T cell persistence and B cell aplasia. Subjects receive preconditioning immunosuppressive chemotherapy of 30-40 mg/kg cyclophosphamide for four days, administered from seven days to three days before the first dose of CAR-expressing cells.

After preconditioning treatment, subjects receive a first dose of CAR-expressing cells by infusion that is less than or equal to about 1×10⁷ cells/kg body weight at day 0. Cells transduced with a GFP-only vector optionally are administered to a group of control animals. Optionally, at seven days after the administration of the of cells, the CAR-expressing cells are detected by flow cytometry and quantitative polymerase chain reaction (qPCR) to measure in vivo proliferation and persistence of the administered cells and B cell aplasia. Administration of PLX3397 (or vehicle alone) is continued up to 21 days after subjects receive the first dose of cells. Recipient animals are monitored for clinical signs and symptoms of one or more toxic outcomes or toxicities such as CRS and neurotoxicity, and data are collected longitudinally to determine CAR T cell expansion and persistence, B cell aplasia, as well as clinical labs of CRS and cytokine levels. Subjects are monitored for symptoms such as fever, hypotension, hypoxia, neurologic disturbances, or an increased serum level of an inflammatory cytokine or C reactive protein (CRP). Optionally, following administration of the first dose, on one or more occasions, blood is obtained from the subjects and the levels of one or more serum factors indicative of toxicity such as neurotoxicity and/or CRS are assessed in the serum by ELISA. The levels of the serum factors are compared to those obtained immediately prior to administration of the first dose.

Factors indicative of therapeutic efficacy, as well as parameters, signs and/or symptoms of or associated with toxicity, assessed in the study, are compared in the various experimental and control groups of animals, over the course of treatment.

Control (GFP-only) cells generally are not observed to persist long-term (e.g., beyond bout one or two weeks) e.g., in the peripheral blood. In this control group no or substantially no clinical signs of CRS or neurologic toxicities are observed. In contrast, the CAR-transduced cells are observed to expand and persist following administration, for example, for greater than one month post-infusion. A reduction in disease burden also is observed in these subjects. Such subjects not pre-treated with the compound also are observed to develop clinical signs and symptoms of CRS and neurological toxicity, e.g., beginning between days 5 to 7 following CAR T cell infusion, for example, with an onset coinciding with maximum CAR T cell expansion and activation. Expansion of CAR-expressing cells in the CFS and/or brain may also be observed, coinciding with the onset of neurotoxicity. In some embodiments, one or more symptoms of toxicity are reduced or prevented in subjects to which the compound has been administered.

### Example 2: Assessment and Comparison of Neuropathology in Different Subjects

Autopsy assessments for neuropathology were performed in four subjects having Acute Lymphoblastic Leukemia (ALL) who developed severe neurotoxicity, including grade 4 or 5 neurotoxicity, and/or cerebral edema following the treatment with therapeutic compositions containing T cells engineered to express a chimeric antigen receptor (CAR). The results generally support the conclusion that brain involvement by B-ALL was not observed to be a factor. Further, in patients with cerebral edema, edema tended to be observed to be vasogenic, not cytotoxic. In patients with cerebral edema, perivascular fibrin and red blood cell extravasation suggested blood brain barrier breakdown. There appeared, however, to be an absence of any remarkable T-cell infiltration, consistent with a conclusion that cerebral edema had not developed as a result of CAR T cell infiltration and/or activation within the brain or CNS.

Further, perivascular and more diffuse patterns of astrocytic and microglial damage/activation was observed in brains of subjects who had developed cerebral edema. The observation was consistent with a conclusion that microglia activation was a contributor to the development of cerebral edema in subjects administered a CAR-T cell therapy. In addition, irreversible damage to astrocytes (clasmatodendrosis) was observed in subjects who developed cerebral edema, contrasted by astrocytic proliferation observed in subjects who developed Grade 4 neurotoxicity. Complete breakdown of the BBB and resulting vasogenic edema was not observed in subjects who developed grade 4 neurotoxicity. In subjects who did not develop cerebral edema, diffuse CD8+ T-cell infiltration that was not consistent with simple reaction to focal injury was observed.

### Example 3: Administration of Anti-CD19 CAR-Expressing Cells to Subjects with Relapsed and Refractory Non-Hodgkin's Lymphoma (NHL)

A. Subjects and Treatment

Therapeutic CAR+ T cell compositions containing autologous T cells expressing a chimeric antigen-receptor (CAR) specific for CD19 were administered to subjects with B cell malignancies. Results are described in this example for evaluation through a particular time-point in an ongoing study for cohort (full cohort) of fifty-five (55) adult human subjects with relapsed or refractory (R/R) aggressive non-Hodgkin's lymphoma (NHL), including diffuse large B-cell lymphoma (DLBCL), de novo or transformed from indolent lymphoma (NOS), primary mediastinal large b-cell lymphoma (PMBCL), and follicular lymphoma grade 3b (FLG3B) after failure of 2 lines of therapy. Among the subjects treated were those having Eastern Cooperative Oncology Group (ECOG) scores of between 0 and 2 (median follow-up 3.2 months). The 55 subjects did not include subjects with mantle cell lymphoma (MCL). No subjects were excluded based on prior allogenic stem cell transplantation (SCT) and there was no minimum absolute lymphocyte count (ALC) for apheresis required.

Outcomes at this time-point for a core subset of the 55 subjects (the subset excluding those subjects with a poor performance status (ECOG 2), DLBCL transformed from marginal zone lymphomas (MZL) and/or chronic lymphocytic leukemia (CLL, Richter's) (core cohort)) were separately assessed.

The demographics and baseline characteristics of the full and core cohort are set forth in Table E1.

TABLE E1
Demographics and Baseline Characteristics

| | FULL | | CORE | |
|---|---|---|---|---|
| Characteristic | N = 55 | | N = 44 | |
| Median Age, years (range) | 61 | (29-82) | 61 | (29-82) |
| ≥65 years, n (%) | 22 | (40) | 17 | (39) |
| Male/Female, n (%) | 38/17 | (69/31) | 28/16 | (64/36) |
| Months from diagnosis, median (range) | 17 | (3-259) | 20 | (8-259) |
| B-NHL Subtype, n (%) | | | | |
| DLBCL, NOS | 40 | (73) | 35 | (80) |
| Transformed DLBCL | 14 | (26) | 8 | (18) |
| Follicular, Grade 3B | 1 | (2) | 1 | (2) |
| Molecular Subtype, n (%) | | | | |
| Double/triple hit | 15 | (27) | 12 | (27) |
| Double expressor | 6 | (11) | 4 | (9) |
| Patient Characteristics, n (%) | | | | |
| Chemorefractory^{†} | 42 | (76) | 34 | (77) |
| ECOG 0-1 | 48 | (87) | 44 | (100) |
| ECOG 2 | 7 | (13) | 0 | |
| Prior lines of therapy, median (range) | 3 | (1-11) | 3 | (1-8) |
| <5 lines of therapy | 44 | (80) | 37 | (84) |
| Any HSCT | 27 | (49) | 22 | (50) |
| Allogeneic | 4 | (7) | 3 | (7) |
| Autologous | 24 | (44) | 20 | (45) |

*SD or PD to last chemo-containing regimen or relapse <12 months after autologous SCT

The therapeutic T cell compositions administered had been generated by a process including immunoaffinity-based enrichment of CD4+ and CD8+ cells from leukapheresis samples from the individual subjects to be treated. Isolated CD4+ and CD8+ T cells were activated and transduced with a viral vector encoding an anti-CD19 CAR, followed by expansion and cryopreservation of the engineered cell populations. The CAR contained an anti-CD19 scFv derived from a murine antibody, an immunoglobulin-derived spacer, a transmembrane domain derived from CD28, a costimulatory region derived from 4-1BB, and a CD3-zeta intracellular signaling domain.

The cryopreserved cell compositions were thawed prior to intravenous administration. The therapeutic T cell dose was administered as a defined cell composition by administering a formulated CD4+ CAR+ cell population and a formulated CD8+ CAR+ population administered at a target ratio of approximately 1:1. Subjects were administered a single or double dose of CAR-expressing T cells (each single dose via separate infusions of CD4+ CAR-expressing T cells and CD8+ CAR-expressing T cells, respectively) as follows: a single dose of dose level 1 (DL-1) containing 5×10⁷ total CAR-expressing T cells (n=30), a double dose of DL1 in which each dose was administered approximately fourteen (14) days part (n=6, including one subject that inadvertently received two DL2 doses via the two-dose schedule, due to a dosing error), or a single dose of dose level 2 (DL-2) containing 1×10⁸ (DL-2) total CAR-expressing T cells (n=18). Beginning at three (3) days prior to CAR+ T cell infusion, subjects received a lymphodepleting chemotherapy with flurabine (flu, 30 mg/m²) and cyclophosphamide (Cy, 300 mg/m²).

B. Safety

Subjects were assessed and monitored for neurotoxicity (neurological complications including symptoms of confusion, aphasia, encephalophathy, myoclonus seizures, convulsions, lethargy, and/or altered mental status), graded on a 1-5 scale, according to the National Cancer Institute Common Toxicity Criteria (CTCAE) scale, version 4.03 (NCI-CTCAE v4.03). Common Toxicity Criteria (CTCAE) scale, version 4.03 (NCI-CTCAE v4.03). See Common Terminology for Adverse Events (CTCAE) Version 4, U.S. Department of Health and Human Services, Published: May 28, 2009 (v4.03: Jun. 14, 2010); and Guido Cavaletti & Paola Marmiroli Nature Reviews Neurology 6, 657-666 (December 2010). Cytokine release syndrome (CRS) also was determined and monitored, graded based on severity.

In 84% of the full cohort subjects, severe (grade 3 or higher) cytokine release syndrome (CRS) and severe neurotoxicity were not observed. Additionally, it was observed that 60% of the full cohort subjects did not develop any grade of CRS or neurotoxicity. No differences in incidence of CRS, neurotoxicity (NT), sCRS, or severe neurotoxicity (sNT) were observed between dose levels. Table E2 summarizes the incidence of cytokine release syndrome (CRS) and neurotoxicity adverse events in patients 28 days after receiving at least one dose of CAR-T cells. As shown in Table E2, no sCRS (Grade 3-4) was observed in any subjects that received a single dose of DL2 or double dose of DLL Severe neurotoxicity or severe CRS (grade 3-4) was observed in 16% (9/55) of the full cohort of subjects and in 18% (8/44) of the subjects in the core subset. 11% (n=6) of subjects received tocilizumab, 24% (n=13) of subjects received dexamethasone. Among the ECOG2 subjects within the full cohort, observed rates of CRS and neurotoxicity were 71% and 29%, respectively.

TABLE E2
Assessment of Presence or Absence of
CRS and Neurotoxicity Adverse Events

| | FULL | | | | |
|---|---|---|---|---|---|
| | All Dose | | | | |
| | Levels | DL1S | DL2S | DL1D^{†} | CORE |
| Safety, N | 55 | 30 | 19 | 6 | 44 |
| sCRS or sNT, n (%) | 9 (16) | 6 (20) | 2 (11) | 1 (17) | 8 (18) |
| CRS or NT, n (%) | 22 (40) | 12 (40) | 7 (37) | 3 (50) | 15 (34) |
| CRS | | | | | |
| Grade 1-2, n (%) | 18 (33) | 10 (33) | 5 (26) | 3 (50) | 12 (27) |
| Grade 3-4, n (%) | 1 (2) | 1 (3) | 0 | 0 | 1 (2) |
| Neurotoxicity | | | | | |
| Grade 1-2, n (%) | 3 (6) | 1 (3) | 2 (11) | 0 | 2 (5) |
| Grade 3-4, n (%) | 9 (16) | 6 (20) | 2 (11) | 1 (17) | 8 (18) |

^{†}Includes one patient treated at DL2 2-dose schedule due to dosing error

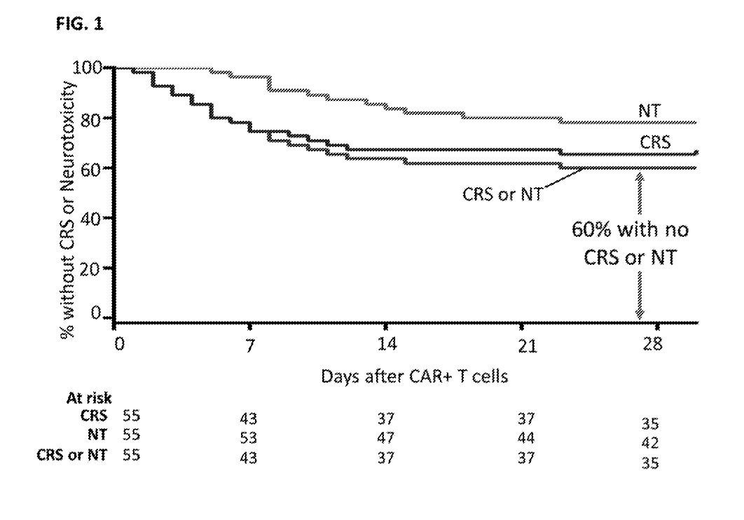

FIG. 1 shows a Kaplan meier curve depicting observed time to onset of CRS and/or neurotoxicity. As shown, the observed median times to onset of CRS and to onset of neurotoxicity were 5 and 11 days, respectively, with only 11% of patients experiencing onset of CRS less than 72 hours after initiation of the administration of the cell therapy. The median time to resolution of CRS and neurotoxicity to Grade 1 or better was 5 and 7 days, respectively. The median time to complete resolution of CRS and neurotoxicity was 5 and 11 days, respectively. The results were consistent with a conclusion that there was a low rate of early onset of any CRS or neurotoxicity in the subjects.

C. Response to Treatment

Subjects were monitored for response, including by assessing tumor burden at 1, 3, 6, 7, 12, 18, and 24 months after administration of the CAR+ T cells. Response rates are listed in Table E3. High durable response rates were observed in the cohort of subjects, which included subjects heavily pretreated or, with poor prognosis and/or with relapsed or refractory disease. For subjects across all doses in the Core (n=44) cohort, the observed overall response rate (ORR) was 86% and the observed complete response (CR) rate was 59%. At three months for the core cohort, the overall response rate (ORR) was 66%; the three-month CR rate was 50% among the core cohort. In the core cohort, the 3 month ORR was 58% (11/19) at dose level 1 and 78% at dose level 2; the 3 month CR rate was 42% (8/19) for dose level 1 and 56% (5/9) for dose level 2, consistent with a suggested dose response effect on treatment outcome. Additionally, the results were consistent with a relationship between dose and durability of response.

TABLE E3
Response

| | FULL | | | | | | | | CORE | | | | | | | |
|---|---|---|---|---|---|---|---|---|---|---|---|---|---|---|---|---|
| | All Dose | | | | | | | | All Dose | | | | | | | |
| | Levels | | DL1S | | DL2S | | DL1D^{c} | | Levels | | DL1S | | DL2S | | DL1D^{a} | |
| Best Overall | 54 | | 30 | | 18 | | 6 | | 44 | | 25 | | 15 | | 4 | |
| Response, N^{a} | | | | | | | | | | | | | | | | |
| ORR, % | 76 | (62, 87) | 80 | (61, 92) | 72 | (47, 90) | 67 | (23, 96) | 86 | (73, 95) | 84 | (64, 95) | 87 | (60, 98) | 100 | (40, 100) |
| (95% CI) | | | | | | | | | | | | | | | | |
| CR, % | 52 | (38, 66) | 53 | (34, 72) | 50 | (26, 74) | 50 | (12, 88) | 59 | (43, 74) | 56 | (35, 76) | 60 | (32, 84) | 75 | (19, 99) |
| (95% CI) | | | | | | | | | | | | | | | | |
| ≥ 3 mos f/u, n^{b} | 41 | | 24 | | 11 | | 6 | | 32 | | 19 | | 9 | | 4 | |
| 3 mo ORR, % | 51 | (35, 67) | 46 | (26, 67) | 64 | (31, 89) | 50 | (12, 88) | 66 | (47, 81) | 58 | (34, 80) | 78 | (40, 97) | 75 | (19, 99) |
| (95% CI) | | | | | | | | | | | | | | | | |
| 3 mo CR, % | 39 | (24, 56) | 33 | (16, 55) | 46 | (17, 77) | 50 | (12, 88) | 50 | (32, 68) | 42 | (20, 67) | 56 | (21, 86) | 75 | (19, 99) |
| (95% CI) | | | | | | | | | | | | | | | | |

DL1S: DL1 1-dose schedule;
DL2S: DL2 1-dose schedule;
DL1D: DL1 2-dose schedule;
^{a}Included patients with event of PD, death, or 28 day restaging scans. Treated patients < 28 days prior to data snapshot were not included.
^{b}The denominator is number of patients who received the CAR T-cell therapy ≥ 3 months snapshot date with an efficacy assessment at Month 3 or prior assessment of PD or death.
^{c}Includes one patient treated at DL2 2-dose schedule due to dosing error

Among the subjects treated six months or greater prior to the particular time-point of the evaluation, of the ten (10) patients that had been in response at three months, 9 (90%) remained in response at six months. At the evaluation time-point, 97% of subjects in the core subset who had responded were alive and in follow-up, median follow-up time 3.2 months.

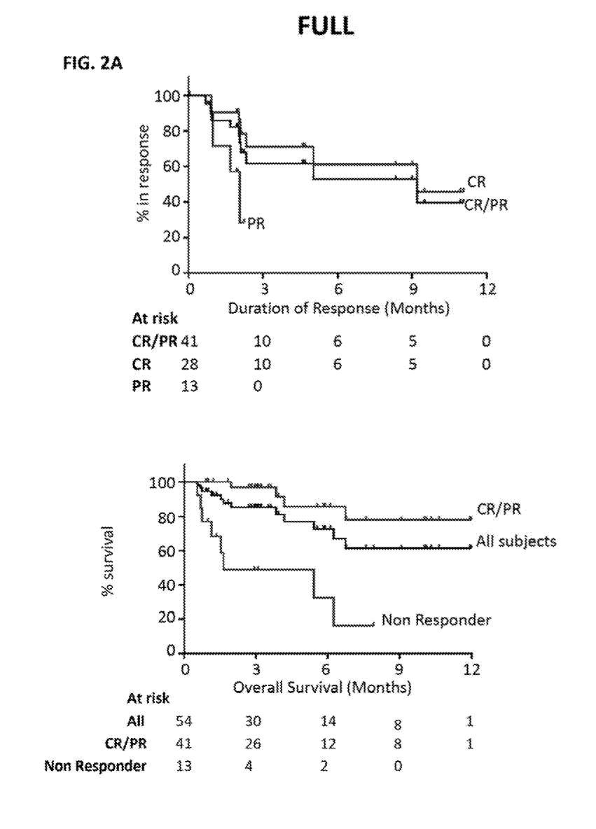

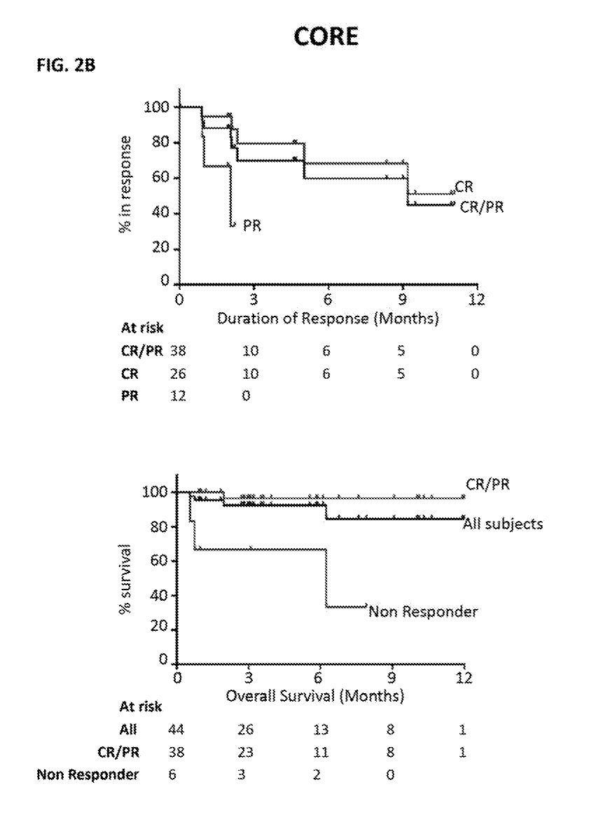

Results for the duration of response and overall survival (grouped by best overall response (non-responder, CR/PR, CR and/or PR)) are shown for full and core cohorts of subjects, in FIGS. 2A and 2B, respectively. As shown, prolonged survival was observed in responders, with increased durability of response in subjects with CRs. All patients in response at three months remained alive at the time of evaluation, although 5/6 subjects with poor performance status (ECOG 2) had expired.

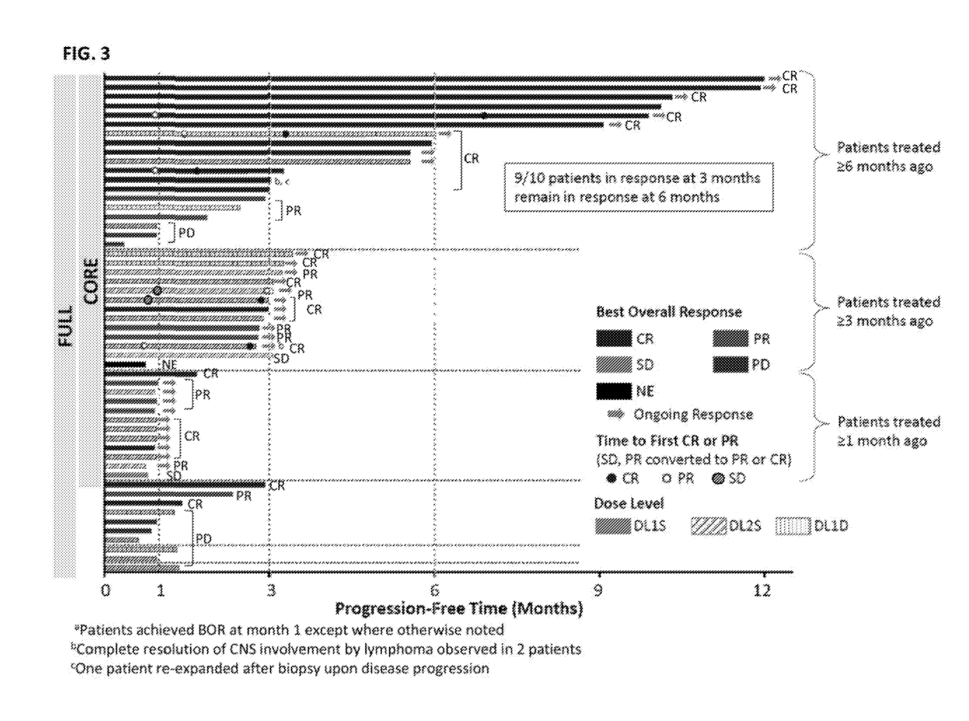

FIG. 3 shows a graph plotting progression-free time (months) for individual subjects within the full and core cohorts. Each bar represents a single patient. Shading indicates best overall response (in each case, unless otherwise indicated, achieved at 1 month); texture indicates dose (solid=dose level 1, single dose; cross-hatched, dose-level 2, single dose; vertical hatched=dose level 1, two-dose). Horizontal arrows indicate an ongoing response. Certain individual subjects were initially assessed (e.g., at 1-month) as exhibiting stable disease (SD) or Partial Response (PR), and were later observed to have achieved a PR (e.g., conversion of SD to PR) or CR. In such cases, shading of the individual patient bar, as noted, indicates best overall response, and dots (same correspondence of shading to response achieved) along each individual subject bar, indicate when each SD, PR, and/or CR was observed to have occurred in the subject. Complete resolution of CNS involvement by lymphoma was observed in two patients. CAR+ cells in one subject were observed to have expanded following biopsy after relapse.

The complete responses in the two DLBCL subjects with CNS involvement were observed without development of any grade of neurotoxicity. These results are consistent with the observation that CAR+ T cells of embodiments provided herein are capable of readily accessing the CNS and exerting effector function to reduce or eliminate CNS tumors, without increasing or without substantially increasing risk of toxicity such as neurotoxicity. In other studies, among subjects having ALL treated with anti-CD19 CAR T cells, no clear correlation has been observed between incidence of neurotoxicity and the presence of CNS leukemia in the brain (which has been observed to respond to such CAR T cell therapy). Thus, whereas neurotoxicity can occur in some contexts following treatment with CAR-T therapies, such neurotoxicity may not necessarily be the result of target expression in the brain or activity of the CAR T cells in the CNS, and may not result from from “on-target” toxicity by the CAR+ T cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID NO. | SEQUENCE | DESCRIPTION |
| 1 | ESKYGPPCPPCP | Spacer |
| | | (IgG4hinge) (aa) |
| | | Homo sapiens |
| 2 | GAATCTAAGTACGGACCGCCCTGCCCCCCTTGCCCT | Spacer |
| | | (IgG4hinge) (nt) |
| | | Homo sapiens |
| 3 | ESKYGPPCPPCPGQPREPQVYTLPPSQEEMTKNQVSLTCLVKGFY | Hinge-CH3 spacer |
| | PSDIAVEWESNGQPENNYKTTPPVLDSDGSFFLYSRLTVDKSRWQ | Homo sapiens |
| | EGNVFSCSVMHEALHNHYTQKSLSLSLGK | |
| 4 | ESKYGPPCPPCPAPEFLGGPSVFLFPPKPKDTLMISRTPEVTCVVVD | Hinge-CH2-CH3 |
| | VSQEDPEVQFNWYVDGVEVHNAKTKPREEQFNSTYRVVSVLTVL | spacer |
| | HQDWLNGKEYKCKVSNKGLPSSIEKTISKAKGQPREPQVYTLPPS | Homo sapiens |
| | QEEMTKNQVSLTCLVKGFYPSDIAVEWESNGQPENNYKTTPPVL | |
| | DSDGSFFLYSRLTVDKSRWQEGNVFSCSVMHEALHNHYTQKSLS | |
| | LSLGK | |
| 5 | RWPESPKAQASSVPTAQPQAEGSLAKATTAPATTRNTGRGGEEK | IgD-hinge-Fc |
| | KKEKEKEEQEERETKTPECPSHTQPLGVYLLTPAVQDLWLRDKA | Homo sapiens |
| | TFTCFVVGSDLKDAHLTWEVAGKVPTGGVEEGLLERHSNGSQSQ | |
| | HSRLTLPRSLWNAGTSVTCTLNHPSLPPQRLMALREPAAQAPVKL | |
| | SLNLLASSDPPEAASWLLCEVSGFSPPNILLMWLEDQREVNTSGF | |
| | APARPPPQPGSTTFWAWSVLRVPAPPSPQPATYTCVVSHEDSRTL | |
| | LNASRSLEVSYVTDH | |
| 6 | LEGGGEGRGSLLTCGDVEENPGPR | T2A |
| | | artificial |
| 7 | RKVCNGIGIGEFKDSLSINATNIKHFKNCTSISGDLHILPVAFRGDS | tEGFR |
| | FTHTPPLDPQELDILKTVKEITGFLLIQAWPENRTDLHAFENLEIIR | artificial |
| | GRTKQHGQFSLAVVSLNITSLGLRSLKEISDGDVIISGNKNLCYAN | |
| | TINWKKLFGTSGQKTKIISNRGENSCKATGQVCHALCSPEGCWGP | |
| | EPRDCVSCRNVSRGRECVDKCNLLEGEPREFVENSECIQCHPECLP | |
| | QAMNITCTGRGPDNCIQCAHYIDGPHCVKTCPAGVMGENNTLVW | |
| | KYADAGHVCHLCHPNCTYGCTGPGLEGCPTNGPKIPSIATGMVG | |
| | ALLLLLVVALGIGLFM | |
| 8 | FWVLVVVGGVLACYSLLVTVAFIIFWV | CD28 (amino |
| | | acids 153-179 of |
| | | Accession No. |
| | | P10747) |
| | | Homo sapiens |
| 9 | IEVMYPPPYLDNEKSNGTIIHVKGKHLCPSPLFPGPSKP | CD28 (amino |
| | FWVLVVVGGVLACYSLLVTVAFIIFWV | acids 114-179 of |
| | | Accession No. |
| | | P10747) |
| | | Homo sapiens |
| 10 | RSKRSRLLHSDYMNMTPRRPGPTRKHYQPYAPPRDFAAYRS | CD28 (amino |
| | | acids 180-220 of |
| | | Accession No. |
| | | P10747) |
| | | Homo sapiens |
| 11 | RSKRSRGGHSDYMNMTPRRPGPTRKHYQPYAPPRDFAAYRS | CD28 (LL to GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Accession No. |
| | | Q07011.1) |
| | | Homo sapiens |
| 13 | RVKFSRSADAPAYQQGQNQLYNELNLGRREEYDVLDKRRGRDPE | CD3 zeta |
| | MGGKPRRKNPQEGLYN ELQKDKMAEA YSEIGMKGER | Homo sapiens |
| | RRGKGHDGLY QGLSTATKDTYDALHMQALP PR | |
| 14 | RVKFSRSAEPPAYQQGQNQLYNELNLGRREEYDVLDKRRGRDPE | CD3 zeta |
| | MGGKPRRKNPQEGLYN ELQKDKMAEA YSEIGMKGER | Homo sapiens |
| | RRGKGHDGLY QGLSTATKDTYDALHMQALP PR | |
| 15 | RVKFSRSADAPAYKQGQNQLYNELNLGRREEYDVLDKRRGRDPE | CD3 zeta |
| | MGGKPRRKNPQEGLYN ELQKDKMAEA YSEIGMKGER | Homo sapiens |
| | RRGKGHDGLY QGLSTATKDTYDALHMQALP PR | |
| 16 | PGGG-(SGGGG)5-P- wherein P is proline, G is | Linker |
| | glycine and S is serine | |
| 17 | GSADDAKKDAAKKDGKS | Linker |
| 18 | EGRGSLLTCGDVEENPGP | T2A |
| 19 | GSGATNFSLLKQAGDVEENPGP | P2A |
| 20 | ATNFSLLKQAGDVEENPGP | P2A |
| 21 | QCTNYALLKLAGDVESNPGP | E2A |
| 22 | VKQTLNFDLLKLAGDVESNPGP | F2A |

1. A method of treatment, comprising administering to a subject having a disease or condition a therapeutic agent for treating a disease or condition, wherein:
administration of the therapeutic agent is or is suspected of being associated with a risk of eliciting a toxic outcome or symptom; and
the subject has been administered, prior to initiation of the therapy, (a) an agent capable of preventing, blocking or reducing or altering an activity or function or phenotype of a microglial cell activity or function or (b) an agent capable of modulating an NRF2 or KEAP1 or HCAR2 or (c) an agent that is an ester of a fumaric acid.

2. The method of claim 1, wherein the prior administration of the agent is in an amount effective to prevent, block or reduce or alter inflammation, oxidative stress response effects, and/or one or more microglial cell activity or function in the subject and/or to promote anti-inflammatory or protective phenotype of an immune cell such as an immune cell in the CNS such as a microglial cell activity or function in the subject.

3. The method of claim 1 or claim 2, further comprising, prior to administering the therapy, administering to the subject the agent capable of preventing, blocking or reducing or altering phenotype of microglial cell activity or function or the agent capable of modulating an NRF2 or KEAP1 or HCAR2 or (c) an agent that is an ester of a fumaric acid.

4. A method of treatment, comprising:
(a) administering to a subject (a) an agent capable of preventing, blocking or reducing or altering an activity or function or phenotype of a microglial cell activity or function or (b) an agent capable of modulating an NRF2 or KEAP1 or HCAR2 or (c) an agent that is an ester of a fumaric acid; and
(b) after the administration in (a), administering to the subject having a disease or condition a therapeutic agent for treating a disease or condition, wherein administration of the therapeutic agent is or is suspected of being associated with a risk of eliciting a toxic outcome or symptom.

5. The method of claim 4, wherein the agent is administered in an amount effective to prevent, block or reduce oxidative stress response effects, and/or one or more microglial cell activity or function in the subject and/or to promote anti-inflammatory or protective phenotype of an immune cell such as an immune cell in the CNS such as a microglial cell activity or function in the subject.

6. The method of any of claims 1-5, wherein the toxic outcome or symptom is associated with neurotoxicity or cytokine release syndrome (CRS).

7. The method of any of claims 1-6, wherein:
the toxic outcome or symptom is associated with severe neurotoxicity and/or is associated with grade 2 or higher or grade 3 or higher neurotoxicity; and/or
the toxic outcome or symptom is associated with severe CRS and/or is associated with grade 2 or higher or grade 3 or higher CRS.

8. The method of any of claims 1-7, wherein the toxic outcome is cerebral edema or is associated with cerebral edema.

9. The method of any of claims 1-8, wherein administration of the agent is started at a time point that is within or within about 1 hour, 2 hours, 6 hours, 12 hours, 24 hours, 3 days, 6 days, 12 days, 15 days, 30 days, 60 days or 90 days or more prior to administration of the therapy.

10. The method of any of claims 1-9, wherein the agent is administered greater than 4 days prior to initiation of the therapy.

11. The method of any of claims 1-10, wherein:
the therapy is not or does not comprise interleukin 2 (IL-2);
the subject has not previously received administration of IL-2 prior to administration of the therapy; or
the subject has not received administration of IL-2 greater than 4 days prior to initiation of the therapy.

12. The method of any of claims 1-11, wherein the agent is not further administered after administration of the therapeutic agent.

13. The method of any of claims 1-12, wherein the method further comprises administering the agent concurrently with or after administration of the therapeutic agent.

14. The method of claim 12, wherein the agent is administered within or within about 1 day, 2 days, 3 days, four days, five days, six days or seven days after administration of the therapeutic agent.

15. A method of treatment, comprising:
(a) administering to a subject having a disease or condition a therapeutic agent for treating a disease or condition, wherein administration of the therapeutic agent is or is suspected of being associated with a risk of eliciting a toxic outcome or symptom of or related to severe CRS or severe neurotoxicity in the subject and/or grade 2 or grade 3 or higher CRS or grade 2 or grade 3 or higher neurotoxicity in the subject; and
(b) administering to the subject (a) an agent capable of preventing, blocking or reducing or altering an activity or function or phenotype of a microglial cell activity or function or (b) an agent capable of modulating an NRF2 or KEAP1 or HCAR2 or (c) an agent that is an ester of a fumaric acid, wherein the agent is administered (i) at a time that is within or within about 1 day, 2 days, 3 days, four days, five days, six days or seven days after administration of the therapeutic agent and/or (ii) at or about or within 24 hours of the subject exhibiting a first sign or symptom indicative of CRS or neurotoxicity after administration of the therapy.

16. The method of claim 15, wherein the agent is administered in an amount effective to prevent, block or reduce oxidative stress response effects, and/or one or more microglial cell activity or function in the subject and/or to promote anti-inflammatory or protective phenotype of an immune cell such as an immune cell in the CNS such as a microglial cell activity or function in the subject.

17. The method of claim 15 or claim 16, wherein the first sign or symptom indicative of CRS or neurotoxicity is a fever.

18. A method of treatment, comprising:
(a) administering to a subject having a disease or condition a therapeutic agent for treating a disease or condition, wherein the therapeutic agent is or is suspected of being associated with a risk of eliciting a toxic outcome or symptom; and
(b) administering to the subject an agent capable of preventing, blocking or reducing or altering an activity or function or phenotype of a microglial cell activity or function or (b) an agent capable of modulating an NRF2 or KEAP1 or HCAR2 or (c) an agent that is an ester of a fumaric acid, wherein the agent is administered at or about or within 24 hours of the subject exhibiting a fever after administration of the therapeutic agent.

19. The method of claim 18, wherein the agent is administered in an amount effective to prevent, block or reduce inflammation, oxidative stress response effects, and/or one or more microglial cell activity or function in the subject and/or to promote anti-inflammatory or protective phenotype of an immune cell such as an immune cell in the CNS such as a microglial cell activity or function in the subject.

20. The method of any of claims 17-19, wherein the fever comprises a temperature of at least or at least about 38.0° C.

21. The method of any of claims 17-20, wherein:
the fever comprises a temperature that is between or between about 38.0° C. and 42.0° C., 38.0° C. and 39.0° C., 39.0° C. and 40.0° C. or 40.0° C. and 42.0° C., each inclusive; or
the fever comprises a temperature that is greater than or greater than about or is or is about 38.5° C., 39.0°, 39.5° C., 40.0° C., 41.0° C., 42.0° C.

22. The method of any of claims 17-21, wherein the fever is a sustained fever.

23. The method of claims 17-22, wherein the fever is a fever that is not reduced or not reduced by more than 1° C. after treatment with an antipyretic and/or wherein the fever has not been reduced by more than 1° C., following treatment of the subject with an antipyretic.

24. The method of claim 15 or claim 16, wherein the first sign or symptom indicative of CRS or neurotoxicity is an altered level of one or more biomarkers in a sample from the subject compared to in the sample prior to administration of the therapeutic agent.

25. The method of claim 24, wherein the sample is a serum or blood sample.

26. The method of claim 24 or claim 25, wherein the sample is obtained or has been obtained from the subject no more than 3 days, no more than 2 days or no more than 1 day after initiation of the therapy or a first administration of the therapeutic agent.

27. The method of any of claims 24-26, wherein the altered level is an increased level of the one or more biomarker, optionally increased greater than or greater than about 1.5-fold, 2-fold, 3-fold, 4-fold, 5-fold, 10-fold, 20-fold or 50-fold.

28. The method of any of claims 24-27, further comprising assessing the sample from the subject for the one or more biomarkers after administration of the therapeutic agent and prior to administration of the agent.

29. The method of any of claims 13-28, wherein administration of the agent is continued after initiation of administration of the therapeutic agent until the risk or suspected risk of a toxic outcome or symptom in the subject from administration of the therapeutic agent has subsided or is not present.

30. A method of ameliorating toxicity induced by or associated with administration of a therapeutic agent, the method comprising:
(a) administering to a subject having a disease or condition a therapeutic agent for treating a disease or condition, wherein the therapeutic agent is or is suspected of being associated with a risk of eliciting a toxic outcome or symptom; and
(b) administering to the subject an agent capable of preventing, blocking or reducing or altering an activity or function or phenotype of a microglial cell activity or function or (b) an agent capable of modulating an NRF2 or KEAP1 or HCAR2 or (c) an agent that is an ester of a fumaric acid, wherein the agent is administered in a dosage regimen until the risk or suspected risk of a toxic outcome or symptom associated with administration of the therapeutic agent has subsided or is not present.

31. The method of claim 30, wherein the agent is administered in an amount effective to prevent, block or reduce inflammation, oxidative stress response effects, and/or one or more microglial cell activity or function in the subject and/or to promote anti-inflammatory or protective phenotype of an immune cell such as an immune cell in the CNS such as a microglial cell activity or function in the subject.

32. The method of claim 30 or claim 31, wherein the agent is administered prior to, simultaneously with and/or subsequent to initiation of administration of the therapeutic agent.

33. The method of any of claims 13-32, wherein the inhibitor is administered for a time period up to 2 days, up to 7 days, up to 14 days, up to 21 days, up to 28 days, up to 35 days, up to 42 days, up to two months, up to three months, up to 6 months or up to 1 year after initiation of the administration of the therapeutic agent.

34. The method of any of claims 29-33, wherein the agent is administered for a time period until:
the grade of CRS or neurotoxicity in the subject is reduced to a lower grade compared to prior to administration of the agent or compared to a preceding time point after administration of the agent; or
a sign or symptom of grade 1 or higher or grade 2 or higher CRS or neurotoxicity is not present or detectable in the subject after administration of the agent.

35. The method of any of claims 1-34, wherein, prior to the administration, the subject has been preconditioned with a lymphodepleting therapy comprising one or more chemotherapeutic agent.

36. The method of any of claims 1-35, further comprising, prior to the administration of the therapeutic agent, administering to the subject a lymphodepleting therapy comprising one or more chemotherapeutic agent.

37. The method of claim 35 or claim 36, wherein the chemotherapeutic agent comprises an agent selected from the group consisting of cyclophosphamide, fludarabine, and/or a combination thereof.

38. The method of claim 37, wherein:
the chemotherapeutic agent is or comprises fludarabine that is administered at a dose of between or between about 1 mg/m² and 100 mg/m², between or between about 10 mg/m² and 75 mg/m², between or between about 15 mg/m² and 50 mg/m², between or between about 20 mg/m² and 30 mg/m², or between or between about 24 mg/m² and 26 mg/m²; and/or
the chemotherapeutic agent is cyclophosphamide that is administered between or between about 20 mg/kg and 100 mg/kg, between or between about 40 mg/kg and 80 mg/kg or between or between about 30 mg/kg and 60 mg/kg.

39. The method of claim 37 or claim 38, wherein the cyclophosphamide is administered once daily for one or two days, and/or the fludarabine is administered daily for 3-5 days.

40. The method of any of claims 37-39, wherein the lymphodepleting therapy comprises administration of cyclophosphamide between or between about 30 mg/kg and 60 mg/kg and administration of fludarabine between or between about 25 mg/m² and 30 mg/m² for three days.

41. The method of any of claims 35-40, wherein the lymphodepleting therapy is initiated at a time that is at least at or about 2 days prior to or is between at or about 2 days and at or about 7 days prior to the administration of the therapeutic agent.

42. The method of any of claims 1-41, wherein the therapeutic agent is an immunotherapy.

43. The method of any of claims 1-42, wherein the therapeutic agent is a T cell therapy or is a T cell-engaging therapy.

44. The method of claim 43, wherein the therapeutic agent is a T cell-engaging therapy comprising a bispecific antibody, wherein at least one binding portion specifically binds to a T cell antigen, optionally CD3.

45. The method of claim 43, wherein the cell therapy is an adoptive cell therapy.

46. The method of any of claims 1-43 and 45, wherein the therapeutic agent is a T cell therapy that is or comprises tumor infiltrating lymphocytic (TIL) therapy or a T cell therapy comprising genetically engineered cells expressing a recombinant receptor that specifically binds to a ligand.

47. The method of claim 45, wherein the T cell therapy is or comprises genetically engineered cells expressing a recombinant receptor that specifically binds to a ligand.

48. The method of any of claims 1-47, wherein the agent capable of preventing, blocking or reducing or altering phenotype of microglial cell activity or function or of modulating an NRF2 or KEAP1 or HCAR2 or that is an ester of a fumaric acid reduces the expression of a microglial activation marker on microglial cells, reduces the level or amount one or more effector molecule associated with microglial cell activation in a biological sample; alters microglial cell homeostasis; decreases or blocks microglial cell proliferation; and/or reduces or eliminates microglial cells.

49. The method of any of claims 1-48, wherein the agent reduces or eliminates microglial cells and the reduction in the number of microglial cells is by greater than 20%, greater than 30%, greater than 40% or greater than 50%, greater than 60%, greater than 70%, greater than 80%, greater than 90%, greater than 95% or greater than 99% compared to the number of microglial cells at a time just prior to initiation of the administration of the agent.

50. The method of any of claims 1-48, wherein:
the agent reduces the expression of a microglial activation marker, optionally CD86 and CD68; and/or
the agent reduces the level or amount of one or more effector molecule, wherein the one or more effector molecule is a optionally or one or more pro-inflammatory mediator, optionally selected from one or more of inducible nitric oxide synthase (iNOS), prostaglandin E(2) (PGE(2)), IL-6, IL-1β, IL-8, CCL2, CXCL10, TNF-α, CCL7, CXCL5, CXCL9, CXCL6, MMP-7, MMP-2, and MMP-9.

51. The method of any of claims 48-50, wherein the biological sample is a brain, serum or plasma sample.

52. The method of any of claims 1-51, wherein the agent that reduces or altering an activity or function or phenotype of a microglial cell activity or function or modulates an NRF2 or or KEAP1HCAR2 or is or comprises an ester of a fumaric acid is selected from an anti-inflammatory agent, an inhibitor of NADPH oxidase (NOX2), a calcium channel blocker, a sodium channel blocker, inhibits GM-CSF, inhibits CSF1R, specifically binds CSF-1, specifically binds IL-34, inhibits the activation of nuclear factor kappa B (NF-κB), activates a CB₂ receptor and/or is a CB₂ agonist, a phosphodiesterase inhibitor, inhibits microRNA-155 (miR-155), upregulates microRNA-124 (miR-124), inhibits nitric oxide production, inhibits nitric oxide synthase, or activates NRF2.

53. The method of any of claims 1-52, wherein the prevention, block or reduction of microglial cell activation or function by the agent is transient and/or is reversible upon discontinued administration of the agent.

54. The method of any of claims 1-53, wherein the agent capable of preventing, blocking or reducing or altering an activity or function or phenotype of a microglial cell activity or function or of modulating an NRF2 or KEAP1 or HCAR2 or that is or comprises an ester of a fumaric acid microglial cell activation or function is a small molecule, peptide, protein, antibody or antigen-binding fragment thereof, an antibody mimetic, an aptamer, or a nucleic acid molecule.

55. The method of claim 54, wherein the agent is selected from minocycline, naloxone, nimodipine, Riluzole, MOR103, lenalidomide, a cannabinoid (optionally WIN55 or 212-2), intravenous immunoglobulin (IVIg), ibudilast, anti-miR-155 locked nucleic acid (LNA), MCS110, PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945, BLZ945, emactuzumab, IMC-CS4, FPA008, LY-3022855, AMG-820, TG-3003, dimethyl dimethyl fumarate, and natalizumab.

56. The method of any of claims 1-55, wherein the agent is an inhibitor of colony stimulating factor 1 receptor (CSF1R).

57. The method of claim 56, wherein the inhibitor transiently inhibits the activity of the CSF1R and/or wherein the inhibition of CSF1R activity is not permanent.

58. The method of any of claims 1-57, wherein:
the inhibitor is selected from PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof;
emactuzumab, IMC-CS4, FPA008, LY-3022855, AMG-820 and TG-3003;
or a combination of any of the foregoing.

59. The method of any of claims 1-58, wherein the inhibitor is PLX-3397.

60. The method of any of claims 1-59, wherein the agent is an inhibitor of nitric oxide synthase.

61. The method of claim 60, wherein the inhibitor of nitric oxide synthase is selected from VAS-203, cindunistat, A-84643, ONO-1714, L-NOARG, NCX-456, VAS-2381, GW-273629, NXN-462, CKD-712, KD-7040, and guanidinoethyldisulfide.

62. The method of any of claims 1-61, wherein the agent is an activator of NRF2 or a function or activity thereof and/or is or comprises an ester of a fumaric acid, and/or modulates or modifies a KEAP1 or HCAR2.

63. The method of claim 62, wherein the activator of NRF2 is dimethyl fumarate.

64. The method of any of claims 1-63, wherein the agent sequesters T cells from the central nervous system.

65. The method of claim 64, wherein the agent modulates a sphingosine-1-phosphate (S1P) receptor.

66. The method of claim 65, wherein the S1P receptor is a S1PR1 and/or a S1PR5.

67. The method of any of claims 64-66, wherein the agent is fingolimod (Gilenya®) or ozanimod (RPC-1063).

68. A method of treatment, comprising administering, to a subject having a disease or condition, a cell therapy for treating a disease or condition, wherein the cell therapy comprises cells that secrete an inhibitor of colony-stimulating factor-1 receptor (CSF1R).

69. The method of claim 68, wherein the cell therapy is a T cell therapy.

70. The method of claim 68 or claim 69, wherein the inhibitor is a peptide, polypeptide or antibody or antigen-binding fragment thereof.

71. The method of any of claims 68-70, wherein the inhibitor is an antibody or antigen-binding fragment thereof.

72. The method of any of claims 68-71, wherein the inhibitor is selected from emactuzumab, IMC-CS4, FPA008, LY-3022855, AMG-820, TG-3003 or is an antigen-binding fragment thereof.

73. The method of any of claims 1-14 and 30-72, wherein the therapeutic agent is administered after administering the agent at a time at which microglial cell activation or function is reduced, blocked or prevented or is likely to be reduced, blocked or prevented in the subject or at a time in which a parameter associated with activity of the agent is altered in the subject.

74. The method of any of claims 1-14 and 30-73, wherein the therapeutic agent is administered after administering the agent at a time at which:
(i) the number of microglial cells is reduced or eliminated in the subject compared to just prior to initiation of administration of the agent; or
(ii) there exists a reduction in the level or amount of a proinflammatory mediator of microglial cell activation in a sample, optionally a brain, serum or plasma sample, from the subject compared to just prior to initiation of administration of the agent;
(iii) the expression of a microglial cell activation marker, optionally CD86 or CD68, is reduced compared to just prior to initiation of administration of the agent;
(iv) there is an increase in the plasma or serum level of CSF-1 or IL-34 compared to just prior to initiation of administration of the agent;
(v) there is a reduction of Kupffer cells and/or an increase in the level or amount of a serum enzyme associated with reduction of Kupffer cells compared to just prior to initiation of administration of the agent;
(vi) there is a reduction in the number of tumor-associated macrophages (TAM) compared to just prior to initiation of administration of the agent; and/or
(vi) there is a decrease in CD14dim/CD16+ nonclassical monocytes in peripheral blood compared to just prior to initiation of administration of the agent.

75. The method of any of claims 1-14 and 30-74, further comprising after administering the agent but prior to administering the therapeutic agent assessing a sample from the subject for a prevention, block or reduction in microglial cell activation or function or for alteration of a parameter associated with activity of the agent.

76. The method of any of claims 1-14 and 30-75, further comprising after administering the agent but prior to administering the therapeutic agent assessing a sample from the subject for one or more of:
(i) a reduction or elimination of microglial cells in the subject compared to just prior to initiation of administration of the agent; or
(ii) a reduction in the level or amount of a proinflammatory mediator of microglial cell activation in a sample, optionally a brain, serum or plasma sample, from the subject compared to just prior to initiation of administration of the agent;
(iii) a reduction in expression of a microglial cell activation marker, optionally CD86 or CD68, compared to just prior to initiation of administration of the agent;
(iv) an increase in the plasma or serum level of CSF-1 or IL-34 compared to just prior to initiation of administration of the agent;
(v) a reduction of Kupffer cells and/or an increase in the level or amount of a serum enzyme associated with reduction of Kupffer cells compared to just prior to initiation of administration of the agent;
(vi) a reduction in the number of tumor-associated macrophages (TAM) compared to just prior to initiation of administration of the agent; and/or
(vi) a decrease in CD14dim/CD16+ nonclassical monocytes in peripheral blood compared to just prior to initiation of administration of the agent.

77. The method of claim 74 or claim 76, wherein the serum enzyme is selected from alanine aminotransferase (ALT), AST, creatine kinase (CK) and LDH.

78. The method of claim 74 or claim 76, wherein the serum cytokine is selected from nitric oxide synthase (iNOS), prostaglandin E(2) (PGE(2)), IL-6, IL-1β, IL-8, CCL2, CXCL10, TNF-α, CCL7, CXCL5, CXCL9, CXCL6, MMP-7, MMP-2, and MMP-9.

79. The method of any of claims 74 and 76-78, wherein the reduction or increase is by greater than or greater than about 1.2-fold, 1.5-fold, 2-fold, 3-fold, 4-fold, 5-fold, 10-fold or more.

80. The method of any of claims 1-79, wherein the toxic outcome or symptom in the subject is reduced or ameliorated compared to a method in which the therapeutic agent is administered to the subject in the absence of the agent.

81. The method of claim 80, wherein the toxic outcome or symptom is associated with neurotoxicity or cytokine release syndrome (CRS), which optionally is severe neurotoxicity or severe CRS.

82. The method of claim 80 or claim 81, wherein the toxic outcome or symptom in the subject at up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent is not detectable or is reduced as compared to a method in which the therapeutic agent is administered to the subject in the absence of the agent.

83. The method of any of claims 80-82, wherein the toxic outcome or symptom is reduced by greater than or greater than about 1.2-fold, 1.5-fold, 2-fold, 3-fold, 4-fold, 5-fold, 10-fold or more.

84. The method of any of claims 1-83, wherein the toxic outcome or symptom is associated with neurotoxicity.

85. The method of claim 84, wherein the neurotoxicity is severe neurotoxicity and/or the neurotoxicity is a grade 3 or higher neurotoxicity.

86. The method of claim 84 or claim 85, wherein the toxic outcome or symptom is associated with grade 3, grade 4 or grade 5 neurotoxicity.

87. The method of any of claims 1-86, wherein the toxic outcome or symptoms is one or more of confusion, delirium, expressive aphasia, obtundation, myoclonus, lethargy, altered mental status, convulsions, seizure-like activity, seizures (optionally as confirmed by electroencephalogram [EEG]), cerebral edema, elevated levels of beta amyloid (Aβ), elevated levels of glutamate, and elevated levels of oxygen radicals, encephalopathy, dysphasia, tremor, choreoathetosis, symptoms that limit self-care, symptoms of peripheral motor neuropathy, symptoms of peripheral sensory neuropathy and combinations thereof.

88. The method of any of claims 84-87, wherein a toxic outcome or symptom of neurotoxicity in the subject at day up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent is not detectable or is reduced as compared to a method in which the therapeutic agent is administered to the subject in the absence of the agent.

89. The method of claim 88, wherein the toxic outcome or symptom of neurotoxicity is reduced by greater than or greater than about 1.2-fold, 1.5-fold, 2-fold, 3-fold, 4-fold, 5-fold, 10-fold or more.

90. The method of any of claims 84-89, wherein the method is such that:
(i) the administration of the therapeutic agent does not induce neurotoxicity in the subject or does not induce severe neurotoxicity in the subject;
(ii) the administration of the therapeutic agent does not induce grade 3 or higher neurotoxicity in the subject, does not induce grade 2 or higher neurotoxicity in the subject or does not induce grade 1 or higher neurotoxicity in the subject;
(iii) based on clinical data, administration of the therapeutic agent does not induce neurotoxcity or does not induce severe neurotoxicity in a majority of subjects so treated; or
(iv) based on clinical data, administration of the therapeutic agent does not result in a toxic outcome or symptom of neurotoxicity greater than grade 3, greater than grade 2 or greater than grade 1 in a majority of the subjects to treated.

91. The method of any of claims 1-90, wherein the toxic outcome or symptom is cerebral edema or is associated with cerebral edema.

92. The method of claim 91, wherein the method is such that:
(i) the administration of the therapeutic agent does not induce cerebral edema in the subject; or
(ii) based on clinical data, a majority of subjects so treated do not exhibit a cerebral edema after the administration of the therapeutic agent.

93. The method of any of claims 1-92, wherein the toxic outcome or symptom is associated with cytokine-release syndrome (CRS).

94. The method of claim 93, wherein the CRS is severe CRS and/or the CRS is grade 3 or higher CRS.

95. The method of claim 93 or claim 94, wherein the toxic outcome or symptom is associated with grade 3, grade 4 or grade 5 CRS.

96. The method of any of claims 93-95, wherein the toxic outcome or symptom is one or more of persistent fever, hypotension, hypoxia, neurologic disturbances, or elevated serum level of an inflammatory cytokine or C reactive protein (CRP).

97. The method of any of claims 93-96, wherein toxic outcome or symptom of CRS in the subject at day up to or up to about day 7, 8, 9, 10, 11, 12, 13, 14, 15, 16, 17, 18, 19, 20, 21, 22, 23, 24, 25, 26, 27, 28, 29 or 30 following initiation of administration of the therapeutic agent is not detectable or is reduced as compared to a method in which the therapeutic agent is administered to the subject in the absence of the agent.

98. The method of claim 97, wherein the CRS is reduced by greater than or greater than about 1.2-fold, 1.5-fold, 2-fold, 3-fold, 4-fold, 5-fold, 10-fold or more.

99. The method of any of claims 93-98, wherein the method is such that:
(i) the administration of the therapeutic agent does not induce CRS in the subject or does not induce severe CRS in the subject;
(ii) the administration of the therapeutic agent does not induce grade 3 or higher CRS in the subject, does not induce grade 2 or higher CRS in the subject or does not induce grade 1 or higher CRS in the subject;
(iii) based on clinical data, administration of the therapeutic agent does not induce CRS or does not induce severe CRS in a majority of subjects so treated;
(iv) based on clinical data, administration of the therapeutic agent does not result in a toxic outcome or symptom of CRS greater than grade 3, greater than grade 2 or greater than grade 1 in a majority of the subjects to treated.

100. The method of any of claims 1-99, wherein the disease or condition is a tumor or a cancer.

101. The method of any of claims 1-100, wherein the disease or condition is a leukemia or lymphoma.

102. The method of any of claims 1-101, wherein the disease or condition is a non-Hodgkin lymphoma (NHL), an acute lymphoblastic leukemia (ALL) or a chronic lymphocytic leukemia (CLL).

103. The method of any of claims 47-102, wherein the recombinant receptor binds to, recognizes or targets an antigen associated with a disease or condition.

104. The method of any of claims 47-103, wherein the recombinant receptor is a T cell receptor or a functional non-T cell receptor.

105. The method of any of claims 47-104, wherein the recombinant receptor is a chimeric antigen receptor (CAR).

106. The method of claim 105, wherein the CAR comprises an extracellular antigen-recognition domain that specifically binds to the antigen and an intracellular signaling domain comprising an ITAM.

107. The method of any of claims 103-106, wherein the antigen is CD19.

108. The method of claim 106 or claim 107, wherein the intracellular signaling domain comprises an intracellular domain of a CD3-zeta (CD3ζ) chain.

109. The method of any of claims 105-108, wherein the CAR further comprises a costimulatory signaling region.

110. The method of claim 109, wherein the costimulatory signaling domain comprises a signaling domain of CD28 or 4-1BB.

111. The method of any of claims 45-110, wherein the cells of the cell therapy are CD4+ or CD8+ T cells.

112. The method of any of claims 45-111, wherein the cells of the cell therapy are autologous to the subject.

113. The method of any of claims 45-112, wherein the cells are allogeneic to the subject.

114. The method of any of claims 45-113, wherein the therapeutic agent is administered in a sufficient dose, without the administration of the agent, to reduce burden of the disease or condition in the subject as indicated by one or more factors indicative of disease burden, wherein the disease burden optionally is a tumor burden.

115. The method of claim 114, wherein the reduction in burden comprises a reduction in total number of cells of the disease in the subject, in an organ of the subject, in a tissue of the subject, or in a bodily fluid of the subject, a reduction in mass or volume of a tumor, and/or a reduction in number and/or extent of metastases.

116. The method of claim 114 or claim 115, wherein:
the dose of cells is sufficient, without administration of the agent, to result in partial remission or complete remission in a majority of subjects so treated with the dose of cells; or
the disease or condition is a cancer and the dose of cells is sufficient, without administration of the agent, to reduce burden of disease from morphological disease to detectable molecular disease and/or minimum residual disease in a majority of subjects so treated; and/or
the disease is a leukemia or lymphoma and the dose of cells is sufficient, without administration of the agent, to reduce the blast cells in the bone marrow to less than or about less than 5%.

117. The method of any of claims 45-116, wherein the cell therapy is administered in a sufficient dose, without the administration of the agent, such that:
there is a maximum concentration or number of cells of the cell therapy in the blood of the subject of at least at or about 10 cells of the cell therapy per microliter, at least 50% of the total number of peripheral blood mononuclear cells (PBMCs), at least at least about 1×10⁵ cells of the cell therapy, or at least 5,000 copies of recombinant receptor-encoding DNA per micrograms DNA; and/or
at day 90 following the initiation of the administration, cells of the cell therapy are detectable in the blood or serum of the subject; and/or
at day 90 following the initiation of the administration, the blood of the subject contains at least 20% cells of the cell therapy, at least 10 cells of the cell therapy per microliter or at least 1×10⁴ recombinant receptor-expressing cells.

118. The method of any of claims 45-117, wherein the cell therapy comprises administration of a dose comprising a number of cells between or between about 0.5×10⁶ cells/kg body weight of the subject and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells·kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

119. The method of any of claims 45-118, wherein:
the dose of cells is a dose that, when administered in the absence of the agent, does, or is likely to, result in severe CRS or grade 3 or higher CRS in the majority of subjects so treated; or
the dose of cells is a dose that, when administered in the absence of the agent, does, or is likely to, result in severe neurotoxicity or grade 3 or higher neurotoxicity in the majority of subjects so treated.

120. The method of any of claims 45-119, wherein the cell therapy is administered at a dose that is higher than a method in which the cell therapy is administered without administering the agent, whereby the agent ameliorates the risk of a toxic outcome to the cell therapy that would occur, or would likely occur, if a similar dose of the cell therapy is administered in the absence of the agent.

121. The method of claim 120, wherein the dose is at least 1.5-fold, 2-fold, 3-fold, 4-fold, 5-fold or 10-fold greater.

122. The method of any of claims 45-121, wherein the cell therapy comprises administration of a dose comprising a number of cells
between about 2×10⁶ cells per kilogram (cells/kg) body weight and about 6×10⁶ cells/kg, between about 2.5×10⁶ cells/kg and about 5.0×10⁶ cells/kg, or between about 3.0×10⁶ cells/kg and about 4.0×10⁶ cells/kg, each inclusive;
between about 1.5×10⁸ cells and 4.5×10⁸ cells, between about 1.5×10⁸ cells and 3.5×10⁸ cells or between about 2×10⁸ cells and 3×10⁸ cells, each inclusive; or
between about 1.5×10⁸ cells/m² and 4.5×10⁸ cells/m², between about 1.5×10⁸ cells/m² and 3.5×10⁸ cells/m² or between about 2×10⁸ cells/m² and 3×10⁸ cells/m², each inclusive.

123. The method of any of claims 45-122, wherein the cell therapy is administered as a single pharmaceutical composition comprising the cells.

124. The method of any of claims 45-123, wherein the cell therapy comprises a dose of cell that is a split dose, wherein the cells of the dose are administered in a plurality of compositions, collectively comprising the cells of the dose, over a period of no more than three days.

125. The method of any of claims 1-124, wherein:
the agent is administered, or each administration of the agent is independently administered, in a dosage amount of from or from about 0.2 mg per kg body weight of the subject (mg/kg) to 200 mg/kg, 0.2 mg/kg to 100 mg/kg, 0.2 mg/kg to 50 mg/kg, 0.2 mg/kg to 10 mg/kg, 0.2 mg/kg to 1.0 mg/kg, 1.0 mg/kg to 200 mg/kg, 1.0 mg/kg to 100 mg/kg, 1.0 mg/kg to 50 mg/kg, 1.0 mg/kg to 10 mg/kg, 10 mg/kg to 200 mg/kg, 10 mg/kg to 100 mg/kg, 10 mg/kg to 50 mg/kg, 50 mg/kg to 200 mg/kg, 50 mg/kg to 100 mg/kg or 100 mg/kg to 200 mg/kg; or
the agent is administered, or each administration of the agent is independently administered, in a dosage amount of from or from about 25 mg to 2000 mg, 25 mg to 1000 mg, 25 mg to 500 mg, 25 mg to 200 mg, 25 mg to 100 mg, 25 mg to 50 mg, 50 mg to 2000 mg, 50 mg to 1000 mg, 50 mg to 500 mg, 50 mg to 200 mg, 50 mg to 100 mg, 100 mg to 2000 mg, 100 mg to 1000 mg, 100 mg to 500 mg, 100 mg to 200 mg, 200 mg to 2000 mg, 200 mg to 1000 mg, 200 mg to 500 mg, 500 mg to 2000 mg, 500 mg to 1000 mg or 1000 mg to 2000 mg, each inclusive.

126. The method of any of claims 1-125, wherein:
the agent is administered, or each administration of the agent is independently administered, in a dosage amount of at least or at least about or about 0.2 mg per kg body weight of the subject (mg/kg), 1 mg/kg, 3 mg/kg, 6 mg/kg, 10 mg/kg, 20 mg/kg, 30 mg/kg, 50 mg/kg, 100 mg/kg or 200 mg/kg; or
the agent is administered, or each administration of the inhibitor is independently administered, in a dosage amount of at least or at least about 25 mg, 50 mg, 100 mg, 200 mg, 400 mg, 500 mg, 600 mg, 800 mg, 1000 mg, 1200 mg, 1600 mg or 2000 mg.

127. The method of any of claims 1-126, wherein the agent is administered daily, every other day, once a week or once a month.

128. The method of any of claims 1-127, wherein the agent is administered daily in a dosage amount of at least or at least about 25 mg/day, 50 mg/day, 100 mg/day, 200 mg/day, 400 mg/day, 500 mg/day, 600 mg/day, 800 mg/day, 1000 mg/day, 1200 mg/day, 1600 mg/day or 2000 mg/day.

129. The method of any of claims 1-128, wherein the inhibitor is administered orally, subcutaneous or intravenously.

130. The method of any of claims 1-129, wherein the subject is a human subject.

131. A combination, comprising:
a first composition comprising genetically engineered cells expressing a recombinant receptor that specifically binds to an antigen; ad
a second composition comprising an inhibitor of colony stimulating factor 1 receptor (CSF1R).

132. The combination of claim 131, wherein the inhibitor reduces the expression of a microglial activation marker on microglial cells, reduces the level or amount one or more effector molecule associated with microglial cell activation in a biological sample; alters microglial cell homeostasis; decreases or blocks microglial cell proliferation; and/or reduces or eliminates microglial cells.

133. The combination of claim 132, wherein the inhibition of CSF-1R and/or the reduction of microglial cell activation by the agent is transient and/or is reversible upon discontinued administration of the agent.

134. The combination of any of claims 131-133, wherein the inhibitor is a small molecule, peptide, protein, antibody or antigen-binding fragment thereof, an antibody mimetic, an aptamer, or a nucleic acid molecule.

135. The combination of any of claims 131-134, wherein the inhibitor is selected from: PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof;
emactuzumab, IMC-CS4, FPA008, LY-3022855, AMG-820 and TG-3003;
or a combination of any of the foregoing.

136. The combination of any of claims 131-135, wherein the inhibitor is PLX-3397.

137. The combination of any of claims 131-136, wherein the recombinant receptor binds to, recognizes or targets an antigen associated with a disease or condition.

138. The combination of any of claims 131-137, wherein the recombinant receptor is a T cell receptor or a functional non-T cell receptor.

139. The combination of any of claims 131-138, wherein the recombinant receptor is a chimeric antigen receptor (CAR).

140. The combination of claim 139, wherein the CAR comprises an extracellular antigen-recognition domain that specifically binds to the antigen and an intracellular signaling domain comprising an ITAM.

141. The combination of any of claims 131-140, wherein the antigen is CD19.

142. The combination of claim 140 or claim 141, wherein the intracellular signaling domain comprises an intracellular domain of a CD3-zeta (CD3ζ) chain.

143. The combination of any of claims 139-142, wherein the CAR further comprises a costimulatory signaling region.

144. The combination of claim 143, wherein the costimulatory signaling domain comprises a signaling domain of CD28 or 4-1BB.

145. The combination of any of claims 131-144, wherein the genetically engineered cells comprise T cells or NK cells.

146. The combination of any of claims 131-145, wherein the cells comprise T cells that are CD4+ or CD8+ T cells.

147. The combination of any of claims 131-146, wherein the cells are primary cells obtained from a subject, optionally a human subject.

148. The combination of any of claims 131-147, wherein the cells are formulated for single dosage administration or multiple dosage administration, which optionally comprises a split dose of cells.

149. The combination of any of claims 131-148, wherein the inhibitor is formulated for single dosage administration or multiple dose administration.

150. A kit comprising the combination of any of claims 131-149 and, optionally, instructions for administering the compositions to a subject for treating a disease or condition.

151. An article of manufacture, comprising:
(a) a pharmaceutical composition comprising engineered immune cells and/or a T cell-engaging therapy; and
(b) instructions for administration of the composition to a subject having a disease or condition, in combination with an agent capable of reducing or preventing or blocking activation or function of microglial cells in the subject.

152. An article of manufacture, comprising:
(a) a pharmaceutical composition comprising an agent capable of reducing or preventing or blocking activation or function of microglial cells; and
(b) instructions for administration of the composition to a subject having a disease or condition, in combination with an agent for treating said disease or condition, which agent comprises an engineered immune cell and/or T cell-engaging therapy.

153. The kit or article of manufacture of any of claims 151-152, wherein the disease or condition is a tumor, optionally a cancer.

154. The kit or article of manufacture of any of claims 151-153, wherein the instructions specify the additional therapeutic agent or therapy is for administration prior to, with or at the same time and/or subsequent to initiation of administration of the engineered immune cell and/or T cell-engaging therapy.

155. The kit or article of manufacture of any of claims 151-154, wherein the instructions further specify the engineered immune cell and/or T cell-engaging therapy is for parenteral administration, optionally intravenous administration.

156. The kit or article of manufacture of any of claims 151-155, wherein the engineered immune cell and/or T cell-engaging therapy comprises primary T cells obtained from a subject.

157. The kit or article of manufacture of claim 156, wherein the T cells are autologous to the subject.

158. The kit or article of manufacture of claim 156, wherein the T cells are allogeneic to the subject.

159. The kit or article of manufacture of any of claims 151-158, wherein the kit or article of manufacture comprises one of a plurality of compositions of the cell therapy comprising a first composition of genetically engineered cells comprising CD4+ T cells or CD8+ T cells, wherein the instructions specify the first composition is for use in with a second composition comprising the other of the CD4+ T cells or the CD8+ T cells, optionally wherein the cells of the first composition and cells of the same composition are from the same subject.

160. The kit or article of manufacture of any of claims 151-159, wherein the agent is a small molecule, peptide, protein, antibody or antigen-binding fragment thereof, an antibody mimetic, an aptamer, or a nucleic acid molecule.

161. The kit or article of manufacture of any of claims 151-160, wherein the agent is selected from: PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof;
emactuzumab, IMC-CS4, FPA008, LY-3022855, AMG-820 and TG-3003;
or a combination of any of the foregoing.

162. The kit or article of manufacture of any of claims 151-161, wherein the agent is PLX-3397.
